SEQUENCE LISTING (5 sequences)

SEQ ID NO: 1 - DNA, 62 bp, Homo sapiens
accatggtgcacctgactcctgaggagaagtctgccgttactgccctgtggggcaaggtgaa

SEQ ID NO: 2 - DNA, 62 bp, Homo sapiens
ttcaccttgccccacagggcagtaacggcagacttctcctcaggagtcaggtgcaccatggt

SEQ ID NO: 3 - DNA, 28 bp, Homo sapiens
gagaagtctgccgttgcaaggtgaagtt

SEQ ID NO: 4 - DNA, 28 bp, Homo sapiens
gagaagagcgctgtgataaagcgccgtc

SEQ ID NO: 5 - DNA, 28 bp, Homo sapiens
gagaagagtgctgtcgcaaagtgcagtt

(19) United States
(12) Patent Application Publication
(10) Pub. No.: US 2020/0392533 A1
(43) Pub. Date: Dec. 17, 2020

(54) IN VIVO GENE EDITING OF BLOOD PROGENITORS

(71) Applicants: President and Fellows of Harvard College, Cambridge, MA (US); Dana-Farber Cancer Institute, Inc., Boston, MA (US)

(72) Inventors: Amy J. Wagers, Cambridge, MA (US); Leo Wang, La Canada Flintridge, CA (US); Jill Goldstein, Cambridge, MA (US)

(73) Assignees: President and Fellows of Harvard College, Cambridge, MA (US); Dana-Farber Cancer Institute, Inc., Boston, MA (US); Dana-Farber Cancer Institute, Inc., Boston, MA (US)

(21) Appl. No.: 16/604,920

(22) PCT Filed: Apr. 11, 2018
(86) PCT No.: PCT/US2018/027197

§ 371 Date: Oct. 11, 2019

Related U.S. Application Data
(60) Provisional application No. 62/484,382, filed on Apr. 11, 2017.
(60) Provisional application No. 62/484,377, filed on Apr. 11, 2017.
(60) Provisional application No. 62/607,305, filed on Dec. 18, 2017.

(51) Int. Cl. C12N 15/86; A61K 48/00; C07K 14/805; C12N 9/22; C12N 15/11; A61P 7/00

(52) U.S. Cl. CPC C12N 15/86; A61K 48/00; C07K 14/805; C12N 2310/20; C12N 15/11; A61P 7/00; C12N 2750/14143; C12N 9/22

(57) ABSTRACT

Disclosed are methods of modifying the genome of HSPCs in vivo by introducing an AAV into a subject transducing a sequence targeting nuclease. In some aspects, the method can be utilized to ascertain causal links between CHIP mutations and age-associated disease. In other aspects, the method can be utilized to treat Sickle cell disease (SCD) and β-thalassemia.

### RELATED APPLICATIONS

This application claims the benefit of U.S. Provisional Application No. 62/484,382, filed on Apr. 11, 2017; U.S. Provisional Application No. 62/484,377, filed on Apr. 11, 2017; and U.S. Provisional Application No. 62/607,305, filed on Dec. 18, 2017. The entire teachings of the above applications are incorporated herein by reference.

### GOVERNMENT SUPPORT

This invention was made with government support under Grant No. AG050395 awarded by the National Institutes of Health. The government has certain rights in the invention. Jaiswal et al., N Engl J Med 371, 2488-2498 (2014)

### BACKGROUND OF THE INVENTION

Perturbed hematopoiesis (blood formation) may be a common driver of age-associated dysfunction. Recent data (Genovese et al., N Engl J Med 372:1071-1072 (2015); Genovese et al., N Engl J Med 37/:2477-2487 (2014); Jaiswal et al., N Engl J Med 371: 2488-2498 (2014); Xie et al., Nat Med 20:1472-1478 (2014)) clearly documenting the emergence in aging humans of clonal outgrowths of blood cells carrying cell-intrinsic somatic gene mutations, and the association of these outgrowths with major age-related pathologies (including hematopoietic cancers, cardiovascular disease and stroke), as well as increased all-cause mortality risk, raise the intriguing possibility that perturbed hematopoiesis (blood formation) may be a common driver of age-associated dysfunction across organ systems.

Sickle cell disease (SCD) and β-thalassemia, known collectively as the β-hemoglobinopathies, result from autosomal recessive mutations in the human HBB gene. HBB encodes the β-globin subunit (HbB) of adult hemoglobin, a heterotetrameric protein composed of 2 α-globin and 2 β-globin subunits that is necessary for the efficient transportation of oxygen throughout the body by red blood cells (RBCs, erythrocytes). Mutations in HBB can occur throughout its sequence, and produce different hematopoietic phenotypes depending on the type, location and consequences for protein expression, and sequence of the mutations affecting each allele. For example, individuals with Sickle Cell Anemia carry two copies of an A→T mutation at the N-terminus of the HBB coding region. This single point mutation causes a change from glutamic acid (GAG) to valine (GTG) at position 6 of the β-globin protein, and thereby produces an abnormal version of β-globin known as hemoglobin S (HbS). HbS has a propensity to misfold and polymerize, particularly at low oxygen tension, and HbS-carrying RBCs become distorted into a crescent or ‘sickle’ shape. Sickled red blood cells have a shortened half-life, and their premature loss leads to chronic and recurrent anemia. In addition, as the distorted sickle cells are more rigid and inflexible than normal RBCs, they can become lodged in small capillaries, causing painful ischemic episodes and long-term damage to critical organs, including the kidneys, lungs and brain.

Mutations in other HBB sequences cause other hemoglobinopathies. For example, mutations in HBB that result in unusually low or absent expression of β-globin cause β-thalassemia. β-globin deficiency in β-thalassemia patients impairs normal development of RBCs from erythroid precursors, leading to anemia, poor oxygenation of tissues, and an increased risk of pathological blood clots.

There are currently no broadly available curative therapies for hemoglobin disorders. Currently, the only curative therapy for SCD or β-thalassemia is allogeneic hematopoietic stem cell transplantation (HSCT), in which a patient's own blood-forming system is replaced by donor cells from an individual with an unaffected HBB gene (6). However, while allogeneic HSCT is successful in >90% of patients who are healthy and have a well matched sibling donor, allogeneic HSCT is inaccessible for many patients due to a lack of appropriate immunologically matched donors, and success rates for patients with alternative donors or patients with end-organ damage and iron overload are significantly lower. In addition, even for patients with well-matched donors, allogeneic HSCT carries with it substantial risks, including a significant risk for development of graft-versus-host disease (GVHD), in which a donor immune response against host cells causes widespread tissue inflammation and damage; graft failure, in which the transplanted cells fail to effectively re-establish hematopoietic cell production; or rejection, in which transplanted cells are destroyed by residual host immune cells. These considerable challenges have limited the widespread application of allogeneic HSCT to β-hemoglobinopathies.

A possible alternative strategy to allogeneic HSCT for the β-hemoglobinopathies has been brought to the fore by recent advances in the field of genome editing. “Genome editing” describes a scientific approach in which engineered programmable nucleases are used to insert, replace or remove segments of DNA within the genome of a living cell or organism (Cheng & Alper, Current Opinion in Biotechnology 30C:87-94 (2014)). In the case of β-hemoglobinopathies, genome editing presents the possibility, explored recently in xenograft systems (Dever et al, Nature 539(7629):384-9 (2016); Hoban et al, Blood 125(17):2597-604 (2015)), of altering the mutant HBB sequences in a patient's own blood-forming cells and then returning these ‘corrected’ cells back to this same patient to support ongoing blood production. This strategy has significant advantages when compared to classical allogeneic HSCT in that (1) every patient can serve as his/her own donor, obviating the need for appropriately matched donors and overcoming immunological barriers to transplantation and GVHD triggers, and (2) editing strategies can be designed that replace the mutant gene with a full length, corrected HBB cDNA, allowing a common targeting strategy to be applied across the spectrum of HBB mutations underlying SCD and β-thalassemia. Importantly, because both SCD and β-thalassemia exhibit autosomal recessive inheritance, only one of the two mutant alleles must be corrected, as individuals carrying at least one unaffected allele typically do not display pathological symptoms.

Current technology for genome editing of blood progenitors comprises ex vivo methods including removal of blood progenitors from a subject, treatment of the blood progenitors to modify their genome, and re-infusion of the modified progenitors. This procedure carries significant risks of graft failure and transplant-related toxicities and requires expensive GMP facilities for handling the ex vivo blood progenitors.

Furthermore, published literature to date reveals significant challenges for retaining robust in vivo engraftment capacity following ex vivo gene editing in HSPCs. For example, an initial study using electroporation of IL2RG directed ZFNs, together with integrase-defective lentiviral vector (IDLV) encoded donor template to enable homology directed recombination (HDR), reported gene targeting rates of 5-12% in cultured human HSPCs, but saw a substantially lower fraction of edited human cells upon analysis of immunodeficient mice transplanted with these HSPCs (Genovese et al, Nature 510(7504):235-40 (2014)). A second study, which likewise used an IDLV encoded donor template or DNA oligonucleotide, together with β-globin directed ZFNs, reported an ˜50-fold reduction in mean levels of genome modification at the β-globin locus after transplant, with several recipient mice lacking any evidence of persistence of β-globin targeted cells (Hoban et al, Blood 125(17):2597-604 (2015)). Finally, while recent studies (Dever et al, Nature 539(7629):384-9 (2016)) have achieved improved ex vivo HSPC transduction rates of up to 26-43%, using an AAV6 encoded donor template together with electroporation of mRNA encoded ZFNs (Wang et al., Nature biotechnology 33(12):1256-63 (2015)) or Cas9 ribonucleoproteins (Dever et al, Nature 539(7629):384-9 (2016)), these groups still saw lower levels of editing in the most primitive CD34+CD133+CD90+ subset of human HSCs (Wang et al., Nature biotechnology 33(12):1256-63 (2015)) and a reduced representation of gene-modified cells in transplanted mice. Furthermore, at least one study found that transduction with an adenoviral vector was cytotoxic to HSPCs (Li et al., Molecular Therapy 21(6):1259-1269; June 2013). Given the problems associated with ex vivo HSPC viral transduction, successful in vivo viral-mediated genome editing of HSPCs seemed highly unlikely.

### SUMMARY OF THE INVENTION

Work described herein surprisingly demonstrates that (1) a dual viral nuclease and donor template system can achieve detectable gene targeting and integration of an anti-sickling β-globin cDNA into the human HBB locus, (2) that this anti-sickling β-globin is appropriately induced upon erythroid differentiation of genome edited HSPCs, and (3) that endogenous tissue stem cells can be transduced by virus in vivo to deliver functional genome editing machinery of relevance to human disease. These surprising results are an improvement over the art and enable gene modification (e.g., HDR-mediated gene modification) in endogenous HSPCs, without a requirement for cell isolation or transplant.

Furthermore, recent data tracing the in vivo clonal dynamics during steady-state hematopoiesis has raised the possibility that the unique function of HSCs as hematopoietic regenerative units may be restricted to the transplant setting, and that endogenous hematopoiesis may be supported largely by a collection of very long-lived, lineage-restricted progenitor cells (25, 26). Given the higher rates of cell division observed for these progenitors, it is possible that they may be more amenable to HDR-based gene editing; however, since they fail to engraft long-term following transplantation (16), they have not been considered as viable targets for ex vivo gene editing approaches. The methods disclosed herein of in vivo editing could overcome this limitation by enabling the modification of such long-acting progenitor cells in situ, where their long-term activity is preserved, thereby providing an alternative or additional source of modified regenerative cells for therapy.

In some aspects, herein is disclosed a strategy for in vivo modification of DNA sequences within endogenous hematopoietic (blood-forming) stem and progenitor cells (HSPCs). This strategy utilizes viral (e.g., AAV-mediated) delivery of sequence targeting nucleases into blood lineage cells in vivo. The delivery virus can be injected directly into the bone marrow or delivered systemically. In some embodiments, the delivery virus is injected intrafemorally.

In some aspects, the invention is directed toward a method for modifying the genome of Hematopoietic Stem and Progenitor Cells (HSPCs) in a subject (e.g., human, mouse), comprising contacting the subject with a virus (e.g., adeno-associated virus (AAV), wherein the virus transduces a nucleic acid sequence encoding a sequence-targeting nuclease into the HSPCs; and modifying the genome of the HSPCs with the sequence targeting nuclease.

In some embodiments, the AAV used in the inventive method is AAV serotype 6, 8 or 9. In some embodiments, the AAV is administered systemically (e.g., intravenously) or is injected into bone marrow.

In some embodiments, the sequence targeting nuclease is a Zinc-Finger Nuclease (ZFN), a Transcription activator-like effector nuclease (TALEN), or a RNA-guided nuclease (e.g., Cas9 nuclease or cpf1 nuclease).

In some embodiments, the method further comprises contacting the subject with a second virus (e.g., AAV) which transduces a nucleic acid sequence encoding one or more gRNAs to a genetic region of interest (e.g., a gene or CHIP).

In some embodiments, the method modifies the genome of CD34−, CD38−, SCA-1+, Thy1.1+/lo, C-kit+, lin−, CD135−, Slamf1/CD150+ hematopoietic stem cells (LT-HSCs). In some embodiments, the method modifies the genome of lineage restricted progenitor cells.

In some embodiments, wherein the genome modification comprises the introduction or correction of a mutation associated with clonal hematopoiesis of indeterminate potential (CHIP). In some embodiments, the modification comprises the introduction or correction of a mutation associated with Sickle cell disease (SCD) or β-thalassemia. In some embodiments, the method treats Sickle cell disease (SCD) or β-thalassemia. In some embodiments, the modification comprises correction of a mutation via homology-directed repair.

In some embodiments, the method further comprises assessing the fate or function of HSPC with genome modification. In some embodiments, the assessment comprises determining if the modification enhances self-renewal of HSPC. In some embodiments, the assessment comprises determining if the modification degrades self-renewal of HSPC. In some embodiments, multiple genomic modifications are made to the HSPC with genome modification. In some embodiments, the genome modification comprises modification of one or more genes associated with biological processes. In some embodiments, the biological processes comprise epigenetic regulation or proteostasis (e.g., autophagy, ubiquitin-proteasome, heat shock response, anti-oxidant response, and/or unfolded protein response).

In some embodiments, the second virus also transduces nucleic acid sequences encoding one or more gRNAs to a cell surface expressed molecule whose loss is non-pathogenic. Disruption of the a cell surface expressed molecule can be used as a marker indicating probable successful targeting of the genetic region of interest as well, since disruption of the cell surface expressed marker requires transduction of the virus having the sequence targeting nuclease and the second virus transducing the gRNAs to a genetic region of interest. The level of cell surface expressed marker on cells should be HIGH in cells containing 2 intact copies of the gene encoding it, LOW in cells containing 1 intact copy and 1 disrupted copy, and ABSENT in cells containing 2 disrupted copies. In some embodiments, the methods of the invention further comprise detection of the level of modification of the genetic region of interest (e.g., one or two alleles). In some embodiments, detection is accomplished by flow cytometry using an antibody specific to cell surface expressed marker.

Some aspects of the invention are directed to a method for in vivo modifying a genetic region of interest in a cell in a subject, comprising contacting the subject with a virus, wherein the virus transduces a nucleic acid sequence encoding a Cas9 nuclease into the cell; contacting the subject with a second virus which transduces a nucleic acid sequence encoding a first set of one or more gRNAs targeting the genetic region of interest and a second set of one or more gRNAs targeting a genetic region encoding or controlling the expression of a cell surface marker; modifying the genetic region of interest with the Cas9 nuclease; and modulating expression of the cell surface marker.

In some embodiments, loss and/or gain of the cell surface marker by the cell is non-pathogenic. In some embodiments, modulating the level of the cell surface marker is non-pathogenic. In some embodiments, the method further comprises detecting the likelihood or degree of modification of the genetic region of interest by detecting a change in the expression of the cell surface marker as compared to a control cell. In some embodiments, a change in the change in the expression of the cell surface marker is detected by immunochemistry (e.g., FACS). In some embodiments, the degree of modulation of the expression of the cell surface marker indicates whether one or both copies of a genetic region of interest are modified by the Cas9 nuclease. In some embodiments, the absence of expression of the cell surface marker indicates that both copies of a genetic region of interest are, or are likely to be, modified by the Cas9 nuclease. In some embodiments, the reduction of expression of the cell surface marker indicates that one copy of a genetic region of interest is, or is likely to be, modified by the Cas9 nuclease. In some embodiments, the high of expression of the cell surface marker indicates that both copies of a genetic region of interest are not, or are likely not, modified by the Cas9 nuclease.

The cell surface marker (e.g., non-pathogenic cell surface marker) is not limited and can be routinely determined in the art. In some embodiments, the cell surface marker is CCR5. The type of cell is also not limited. In some embodiments, the cell is any cell described herein. In some embodiments, the cell is an HSPC.

Some aspects of the invention are directed to a method of screening for genetic regions coding for regulators of hematopoietic stem cell (HSC) self-renewal and/or differentiation, comprising contacting an HSC in vivo with a virus, wherein the virus transduces a nucleic acid sequence encoding a sequence targeting nuclease into the HSC; modifying a genetic region of the HSC with the sequence targeting nuclease; assessing the self-renewal and/or differentiation of the modified HSC; wherein if modification of the genetic region modulates self-renewal and/or differentiation of the HSC then the genetic region is identified as coding for a regulator of hematopoietic stem cell (HSC) self-renewal and/or differentiation. In some embodiments, the genetic region is a gene linked to dysregulated hematopoiesis and/or hematopoietic malignancy, or is linked to variations in HSC self-renewal activity. In some embodiments, the virus is adeno-associated virus (AAV).

In some embodiments, the AAV is AAV serotype 6, 8, 9 or 10. In some embodiments, the virus is administered intravenously or is injected into bone marrow. In some embodiments, the sequence targeting nuclease is a Zinc-Finger Nuclease (ZFN), a Transcription activator-like effector nuclease (TALEN), or a Cas9 nuclease.

In some embodiments, the methods further comprise contacting the subject with a second virus which transduces a nucleic acid sequence encoding one or more gRNAs, wherein the one or more gRNA target the genetic region. In some embodiments, the second virus is an AAV.

Some aspects of the invention are directed to a composition for modifying the genome of Hematopoietic Stem and Progenitor Cells (HSPCs) in a subject comprising a virus encoding a sequence targeting nuclease (e.g., targetable nuclease) as described herein.

Some aspects of the invention are directed to a composition for modifying the genome of Hematopoietic Stem and Progenitor Cells (HSPCs) in a subject comprising a virus encoding a sequence targeting nuclease (e.g., Cas9) as described herein and a second virus encoding one or gRNAs as described herein.

Some aspects of the invention are directed to a composition for modifying a genetic region in vivo comprising a virus encoding a sequence targeting nuclease (e.g., Cas9) as described herein and a second virus encoding one or gRNAs targeting the genetic region and one or more gRNAs targeting expression of a cell surface molecule as described herein.

The above discussed, and many other features and attendant advantages of the present inventions will become better understood by reference to the following detailed description of the invention.

### BRIEF DESCRIPTION OF THE DRAWINGS

The patent application contains at least one drawing executed in color. Copies of this patent application publication with color drawings will be provided by the Office upon request and payment of the necessary fee.

FIG. 1 is an illustration showing that aging is the single biggest risk factor for many diseases, including diabetes, dementia, osteoporosis, heart disease, stroke, cancer, kidney failure, loss of skeletal muscle mass and function, vision loss and infection.

FIG. 2 is a graph and heat map showing that global population aging will dramatically increase the incidence and health burden of aging-related disease dysfunctions.

FIG. 3 is an illustration showing that there are many age related disorders needing therapeutic solutions.

FIG. 4 is an illustration showing that age related disorders may be treated with therapies to each disorder or groups of disorders indicated by color similarity or identity.

FIG. 5 is an illustration showing that age related disorders may be treated by treating the underlying mechanism of aging.

FIG. 6 is an illustration asking whether therapies for these diseases can target the “root cause”—common mechanisms regulating the aging process—to develop common interventions for age-associated diseases.

FIG. 7 shows a graph showing that the prevalence of somatic mutations in peripheral blood cells increases with age. Prevalence of Somatic Mutations, According to Age. Colored bands, in increasingly lighter shades, represent the 50th, 75th, and 95th percentiles.

FIG. 8 is a bar graph showing that prevalent age-related somatic mutations occur in specific genes that are also mutated in blood cell cancers.

FIG. 9 is a graph showing that clonal hematopoiesis is associated with increased risk of blood malignancies.

FIG. 10 is a graph showing that clonal hematopoiesis is associated with increased risk of age-related disease.

FIG. 11 shows the effect of Somatic Mutations on All-Cause Mortality. A forest plot of the risk of death from any cause associated with having a somatic clone, among participants from the JHS cohort, the Ashkenazi cohort of the Longevity Genes Project (UA), the MEC, the Finland-United States Investigation of NIDDM Genetics Study (FUSION) cohort, and the Botnia Study cohort. The left panel includes data from participants who were younger than 70 years of age at the time of DNA ascertainment, and the right panel data from participants who were 70 years of age or older.

FIG. 12A-12C shows that TET2 deficiency in macrophages promotes inflammation and aggravates atherosclerosis. (FIG. 12A) Ldlr−/− Mye-Tet2-KO mice (LysM-Cre+ Tet2flox/flox BMT) and WT controls (LysM-Cre− Tet2flox/flox BMT) were fed a HFHC diet for 10 weeks. (FIG. 12B) qRT-PCR analysis of TET2 transcript levels in BM derived macrophages isolated from Mye-Tet2-KO mice and WT controls (n=6 mice per genotype). (FIG. 12C) Aortic root plaque size. Representative images of H&E-stained sections are shown; atherosclerotic plaques are delineated by dashed lines. Scale bars, 100 mm.

FIG. 13 is an illustration of Research Goals: Develop a system to enable intentional disruption in blood stem/progenitor cells of endogenous genes implicated in the emergence of clonal hematopoiesis. Use this system (the present invention) to monitor the impact of individual (or multiple) gene mutations on aging phenotypes. Use this system (the present invention) to discover new potential targets.

FIG. 14 is an illustration of in vivo gene editing using CRISPR/Cas9 delivered via Adeno Associated Virus (AAV). R=G A (purine)

FIG. 15 illustrates Intrafemoral delivery of AAV-CRISPR transduces and targets genes in endogenous hematopoietic stem and progenitor cells (HSPCs).

FIG. 16 illustrates Systemic delivery of AAV-CRISPR transduces and targets genes in endogenous hematopoietic stem and progenitor cells (HSPCs).

FIG. 17 illustrates AAV-CRISPR transduces and targets genes in endogenous hematopoietic stem and progenitor cells (HSPCs).

FIG. 18 illustrates using AAV-CRISPR to model clonal hematopoiesis. In vivo AAV-CRISPR approach allows introduction of CH-relevant mutations in endogenous HSPCs and at physiologically relevant frequencies.

FIG. 19 illustrates using AAV-CRISPR to model clonal hematopoiesis.

FIG. 20 illustrates outcomes for AAV-CRISPR model studies of clonal hematopoiesis.

FIG. 21A-21E shows in vivo transduction and genome editing of hematopoietic progenitors with AAV-encoded nuclease. (FIG. 21A) Experimental design. AAVs carrying a nuclease (in this case, Cre) targeting the loxP sequences of the Ai9 cassette were injected into Ai9 transgenic mice bearing a lox-STOP-lox-tdTomato cassette. (FIG. 21B) Flow cytometric analysis of bone marrow (BM), spleen and blood 4 weeks after AAV administration revealed genomic excision of the STOP cassette, and subsequent tdTomato expression in mature lymphoid and myeloid cells (not shown), as well as hematopoietic stem cells (quantified as mean+/−SD; n=2-4 mice per serotype). (FIG. 21C) Subsequent transplant of tdTomato+ cells from AAV-injected donors into irradiated CD45.1+ recipients revealed long-term, multi-lineage reconstitution by tdTomato+ cells, confirming permanent genome modification of HSCs via this methodology. Lineage designations: T=T cell; B=B cell; M=Monocyte; N=Neutrophil. In the studies proposed herein, Cre will be replaced with Cas9+ guide RNAs targeting genes of interest (e.g., Dnmt3a, Tet2 and/or Asxl1) for HSC regulation, using a similar system to that we applied previously to edit genes in vivo (2). (FIG. 21D-FIG. 21E) Summary of two independent experiments targeting endogenous LT-HSCs with AAV-Cre of the indicated serotypes. Middle column: Flow cytometry analysis of % tdTomato+ HSPCs in mice administered different AAV serotypes harboring Cre via (FIG. 21D) intrafemoral or (FIG. 21E) intravenous injection. Right column: Fraction of mice transplanted with bone marrow cells from the AAV-Cre injected Ai9 animals that contain tdTomato+ blood cells (FIG. 21D) 6 months or (FIG. 21E) 2 months post-transplant.

FIG. 22 shows the prevalence of clonal hematopoiesis per decade.

FIG. 23 illustrates recurrent mutations identified from exome sequencing of human peripheral blood cells. Figure compiles data from 3 recent studies (3-5).

FIG. 24A-24E-(FIG. 24A) is a Schematic depicting the Ai9 allele, and design of saCas9 gRNAs that direct Cas9 excision of the STOP cassette to enable TdTomato expression. (FIG. 24B) Dual AAV system for systemic delivery of saCas9 and Ai9 gRNAs. (FIG. 24C) Representative FACS plots of tdTomato expression among Pax7-ZsGreen+ muscle stem cells isolated from Pax7-ZsGreen+/−;mdx;Ai9 mice treated systemically with vehicle (left), AAV-Cre (middle) or AAV-Ai9 CRISPR (right). (FIG. 24D) Quantification of FACS data showing % of muscle stem (satellite) cells expressing tdTomato after systemic injection of Pax7-ZsGreen+/−;mdx;Ai9 mice with AAV-Ai9-CRISPR. Individual data points overlaid with mean±SD; vehicle (n=3), AAV-Cre (n=4) AAV Ai9 CRISPR (n=5). (FIG. 24E) Muscle stem cells isolated from a dystrophic muscle injected intramuscularly with AAV-Ai9-CRISPR were FACSorted and expanded in culture for 2 wks., and then transplanted into cardiotoxin-preinjured recipient mdx mouse muscle (Left). Ten days later, muscles were harvested for fluorescence imaging analysis (Laminin, green; tdTomato, red). Detection of tdTomato+ donor-derived myofibers (left) demonstrates the capacity of gene-edited stem cells to engraft and contribute to muscle regenerative responses in vivo. TdTomato+ myofibers were not detected in muscles injected with vehicle only (right). Scale bar: 100 μm. Data reproduced from (2).

FIG. 25A-25C—illustrates in vivo transduction and genome modification of mouse HSPCs. Adult (8-10 wks) Ai9 transgenic mice, harboring the LSL-tdTomato transgene, were injected systemically (FIG. 25A, FIG. 25B) or intrafemorally (FIG. 25C) with AAV-Cre vectors of the indicated serotypes (2-4e12 vector genomes (vg) per recipient). 4 weeks later, mice were sacrificed for flow cytometric analysis of tdTomato expression (an indicator of nuclease activity) in Lin-c-kit+Sca1+(LSK) progenitors (FIG. 25A) and immunophenotypic HSCs (CD150+CD48-LSK) (FIG. 25B). Tdtomato+ cells were also transplanted into irradiated CD45 congenic recipients and analyzed for multi-lineage hematopoietic engraftment 16 weeks later (FIG. 25C).

FIG. 26 shows transduction of immunophenotypic LT-HSCs by AAV-Cre.

FIG. 27 shows transduction of immunophenotypic LT-HSCs by AAV-Cre and nucleic acid sequences for two vector AAV transduction of saCas9 and two gRNAs.

FIG. 28A-28C-(FIG. 28A) is a schematic showing in vivo AAV-Cre editing of mdx-Ai9 mice to produce tdTomato+LT-HSC followed by injection of tdTomato+LT-HSC into irradiated CD45.1 host. (FIG. 28B) Bar graph showing % tdTomato LT-HSCs after in vivo AAV-Cre editing of mdx-Ai9 mice. (FIG. 28C) Graph showing % tdTomato LT-HSCs donor cells.

FIG. 29A-29C illustrates in vivo transduction and genome modification of mouse HSPCs. Ai9 transgenic mice, harboring the LSL-tdTomato transgene, were injected systemically (FIG. 29A, FIG. 29B) or intrafemorally (FIG. 29C) with AAV-Cre vectors of the indicated serotypes. Four weeks later, mice were sacrificed for flow cytometric analysis of tdTomato expression (an indicator of Cre-mediated nuclease activity) in Lin-c-kit+Sca1+ (LSK) progenitors (FIG. 29A) and immunophenotypic HSCs (CD150+CD48-LSK) (FIG. 29B). Tdtomato+ cells were also transplanted to CD45 congenic recipients (FIG. 29C).

FIG. 30A-30C shows in vivo transduction and genome modification of mouse HSPCs. Ai9 transgenic mice, harboring the LSL-tdTomato transgene, were injected systemically (FIG. 30A, FIG. 30B) or intrafemorally (FIG. 30C) with AAV-Cre vectors of the indicated serotypes. Four weeks later, mice were sacrificed for flow cytometric analysis of tdTomato expression (an indicator of Cre-mediated nuclease activity) in Lin-c-kit+Sca1+ (LSK) progenitors (FIG. 30A) and immunophenotypic HSCs (CD150+CD48-LSK) (FIG. 30B). Tdtomato+ cells were also transplanted to CD45 congenic recipients (FIG. 30C).

FIG. 31 illustrates FACS dot plots showing spleen mature lineages for tdTomato+ cells.

FIG. 32 shows bar graph showing long term (16w), multi-lineage engraftment from AAV-Cre transduced BM cells.

FIG. 33A-33B illustrates HBB Gene targeting machinery. (FIG. 33A) Schematic diagram of Left and Right TALENs (L4 and R4) targeting the HBB locus near the sickle mutation site (highlighted ‘a’). TALENs were designed and tested in the Porteus laboratory (4). (FIG. 33B) Schematic diagram of the MDM20 and GW15 donor templates for HR utilized in our preliminary studies, which enable targeting of the HBB locus. MDM20 allows integration of GFP utilizing the endogenous β-globin start codon (ATG) to drive its expression. GW15 allows integration of an anti-sickling version of the human β-globin cDNA (8-11), similarly controlled by the endogenous β-globin promoter. GW15 also allows β-globin promoter dependent expression of citrine fluorescent protein, encoded 3′ of the anti-sickling β-globin and separated by a self-cleaving 2A peptide sequence and selection marker (P140K MGMT) which is expressed ubiquitously from the ubiquitin C (UBC) promoter. Integration of GW15 in the HBB locus results in anti-sickling β-globin and citrine expression ONLY in β-globin expressing cells (i.e., differentiated erythroid cells) and ubiquitous expression of P140K MGMT. Integration in non-HBB loci could theoretically also result in citrine expression if near to active promoter elements. Thus, DNA sequencing of citrine+ cells will be used to confirm integration events at HBB and distinguish from (presumably rare) integration at other non-homologous locations.

FIG. 34A-34B illustrates TALEN-catalyzed genome modification at the HBB locus in human erythroid cells derived from primary CD34+ HSPCs. (FIG. 34A) Experimental design. One million BM CD34+ HSPCs from healthy human donors were nucleofected with plasmid DNA encoding the β-globin donor template MDM20 (4 ug, see FIG. 36) or MDM20 together with the HBB-targeting L4/R4 TALEN pair (1 ug each TALEN). Transfected and untransfected cells (as control, not shown) were placed in erythroid differentiation medium (StemSpan media containing EPO, IL-3, IL-6, and SCF) for 7-12 days, and then harvested for flow cytometry. (FIG. 34B) Cultures were stained for erythroid markers, including CD235a (glycophorin A, GPA) and analyzed for green fluorescent protein (GFP) expression within the GPA+ β-globin expressing erythroid subset. FACS data are shown as dot plots of side scatter (SSC, Y-axis) versus GFP (X-axis) and previously gated to show only viable GPA+ erythroid cells (left plots and data not shown). Data representative of >8 independent experiments with different human donors.

FIG. 35A-35C shows MGMT-mediated enrichment of stably modified human HSPCs. (FIG. 35A) Experimental design. One million human BM CD34+ HSPCs were electroporated with 1 ug of donor GFP template (GW15) or 1 ug of GW15 and 0.5 ug each of the β-globin-specific TALENs L4 and R4. Cells were cultured in erythroid media, and split or treated with BG+BCNU according to the indicated time line. A subset of cells was analyzed by flow cytometry for CD235a (GPA) and citrine expression at the time of splitting (days 3, 6, and 9) and at the termination of the experiment (day 14). (FIG. 35B) Prior to drug selection (day 3), cells co-transfected with GW15+L4/R4 TALENs included a small fraction (0.2%) of citrine+ GPA+ cells (red arrow). (FIG. 35C) The frequency of citrine+ GPA+ cells in cultures co-transfected with GW15+L4/R4 TALENs increased after each round of drug selection, to 1.89% at day 6, 19.3% at day 9, and 36.2% at day 14 (red boxes), for a total enrichment of 180-fold after 3 rounds of selection. No citrine expression was detected in any non-erythroid (GPA-negative) cells (data not shown). Data are representative of 4 experiments with 4 independent human donors. Different levels of citrine expression in (FIG. 35B) and (FIG. 35C) reflect different stages of asynchronous erythroid maturation in these cultures.

FIG. 36 shows DNA sequence analysis by SMRT sequencing confirms correct targeting at the HBB locus following co-transfection of β-globin TALENs+donor template. Wild-type (endogenous sequence) reads shown in black; gene targeted reads (with the expected integrated sequence) in white. % indicates percentage of reads showing sequence expected from integration of the donor cassette into the HBB locus. ND, no gene targeted reads detected.

FIG. 37 shows detection of anti-sickling β-globin mRNA in human HSPCs after cotransfection with β-globin TALENs+donor template. (top) Unique DNA ‘signatures’ allowing discrimination of endogenous β- and δ-globin transcripts from the highly homologous anti-sickling β-globin mRNA introduced by TALEN-directed HR at the β-globin locus. (bottom) Table indicating the percentage of RNA-sequencing reads attributable to endogenous β-globin, TALEN-delivered anti-sickling β-globin or endogenous δ-globin in untransfected HSPCs (untransfected), HSPCs transfected with donor only (Donor) or HSPCs co-transfected with TALENs+donor before (Pulse=0) or after 1 round (Pulse=1), 2 rounds (Pulse=2) or 3 rounds (Pulse=3) of BG/BCNU drug selection. Cells were not sorted based on citrine positivity or GPA expression prior to RNA sequencing. Data are representative of one of four independent sequencing experiments using four different healthy human donors.

FIG. 38 shows flow cytometric and epifluorescence analysis of citrine expression by HSPCs from an SCD patient. Umbilical cord blood cells were enriched for CD34+ cells by magnetic selection and then nucleofected with L4-R4 TALENs+GW15 donor plasmid. Mock transfected HSPCs from the same patient serve as control. Samples were cultured without selection (mock and unselected columns) or with selection using a single (d5) or double (d10) pulse of O6BG and BCNU. 92-95% of the cells analyzed in these cultures were CD71+GPA+ erythroid cells. Citrine expression, detected by FACS (top) or epifluorescence (bottom) indicates proper integration of the donor construct in the HBB locus.

FIG. 39A-39B illustrates CRISPR-Cas9 targeting of HBB. (FIG. 39A) T7E1 assay of PCR products amplified from K562 cells nucleofected with plasmid encoding Streptococcus pyogenes (Spy) Cas9 and Spy gRNA (R66), which uses an “NGG” PAM (A) or Staphylococcus aureus Cas9 (Sau) and Sau gRNA Sa_12, which uses an “NNGGR(T)” PAM Both R66 and Sa_12 target the sickle cell mutation in exon 1. T7EI sensitive bands in lanes A and B, which represent replicate experiments, indicate the presence of modified HBB alleles harboring small insertions or deletions. (FIG. 39B) Frequency of modified alleles in treated K562 cells.

FIG. 40 is a Schematic of AAV vector (AAV-GW25) for delivery of Sa_12 HBB gRNA and donor template for HDR at the HBB locus.

FIG. 41A-41B shows AAV-CRISPR/Cas9 mediates disruption of an endogenous gene in the genome of endogenous hematopoietic stem cells. (FIG. 41A) Hemizygous CAAGS-eGFP mice, containing a single transgenic allele encoding ubiquitous GFP expression were injected with AAV-CRISPR particles (serotype 8) targeting disruption of the GFP transgene. Three weeks later, bone marrow cells from the AAV-CRISPR injected mice were transplanted into wild-type recipients. (FIG. 41B) ⅓ of recipient mice showed multi-lineage hematopoietic reconstitution with GFP− blood cells, indicating disruption in blood reconstituting hematopoietic stem and progenitor cells (HSPCs) of the genomically encoded GFP transgene by the AAV8-delivered gene editing complexes. In contrast, 100% of recipients of bone marrow cells from non-targeted mice showed engraftment with GFP+ cells. Data show peripheral blood cell analysis at 8 weeks after transplant of WT (top left) and GFP control cells (top right) or cells from AAV8-CRISPR injected mice (bottom), including one animal reconstituted by non-disrupted (GFP+) HSPCs (bottom left) and one reconstituted by disrupted (GFP−) HSPCs (bottom right).

FIG. 42 illustrates components for establishing optimal viral serotypes and titers for disrupting known aging-relevant target genes in endogenous mouse and human (xenografted) HSCs.

FIG. 43 illustrates components for multiplexed screening strategies to identify gene targets that enhance self-renewal of endogenous human HSCs.

FIG. 44 illustrates unique proteostasis genes downregulated in aged vs. young HSPCs.

FIG. 45 is an illustration of pathways that control stem cell self-renewal endogenously.

FIG. 46A-46B-(FIG. 46A) illustrates a Mouse Reporter System with nucleotide sequences for SaCas9 and hybrid reporter for Ai9-Dmd. (FIG. 46B) is a human reporter system with nucleotide sequences for SaCas9 and hybrid reporter for Gene of Interest (GOI).

FIG. 47 is a schematic showing mouse reporter system for CRISPR-Cas9 in vivo editing resulting in expression of tdTomato. Two AAV vectors used, one for SaCas9 and one for two Ai9 gRNAs.

FIG. 48 illustrates a graph of hypothetical data from FACS of human reporter system showing populations of cells expressing low levels, medium levels or high levels of a reporter protein.

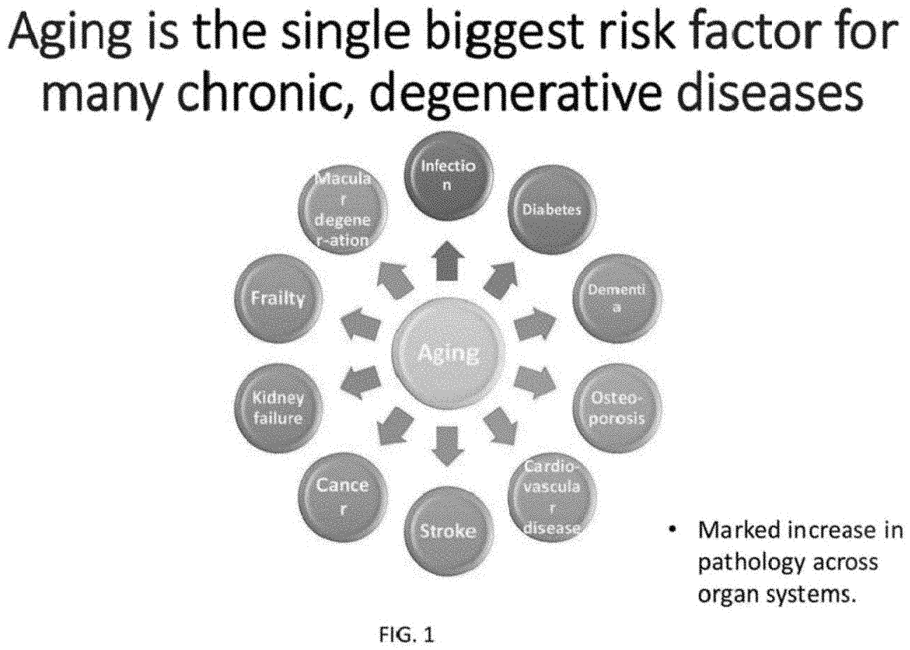

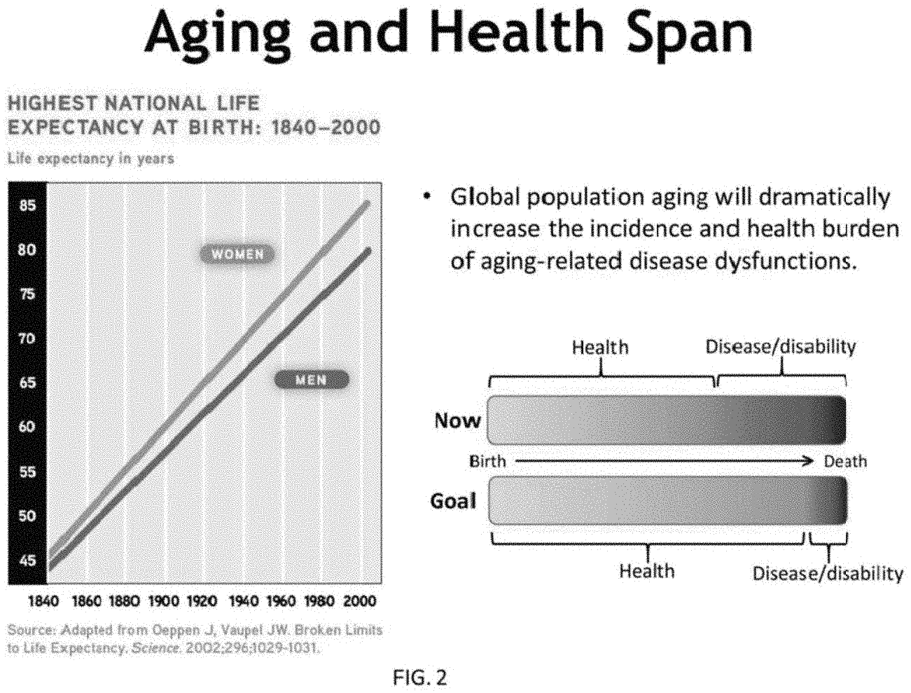

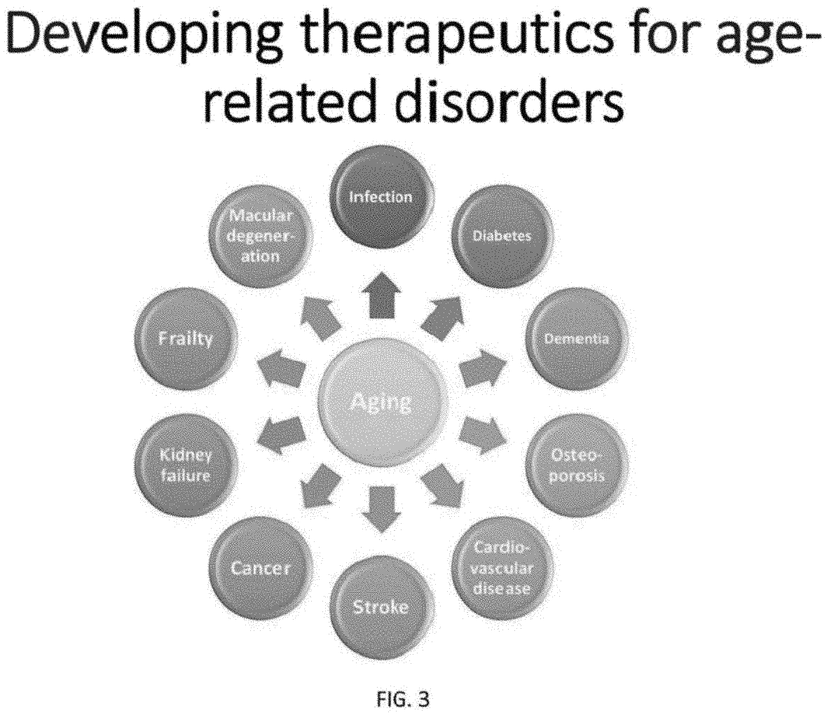

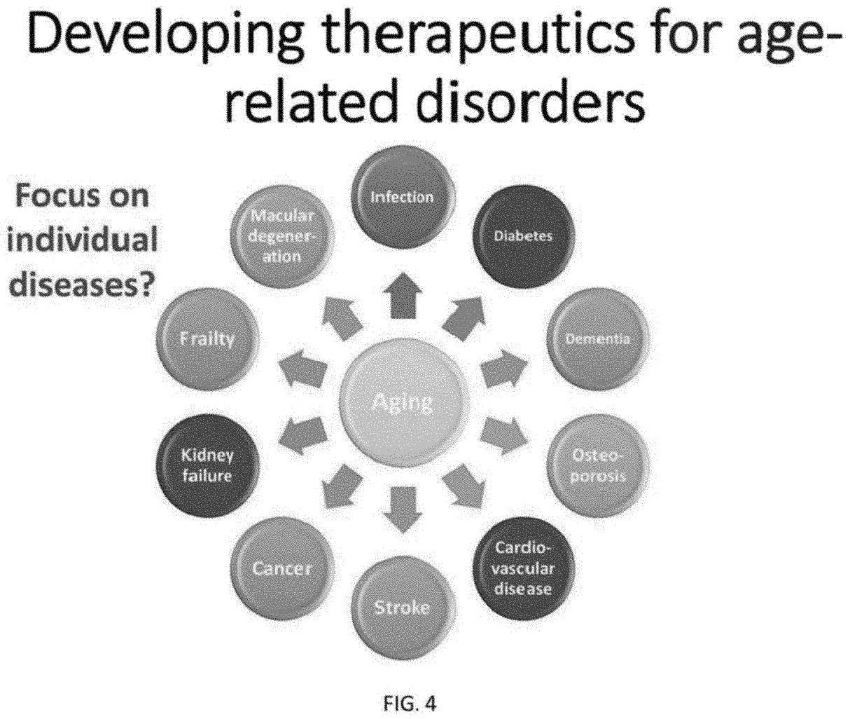

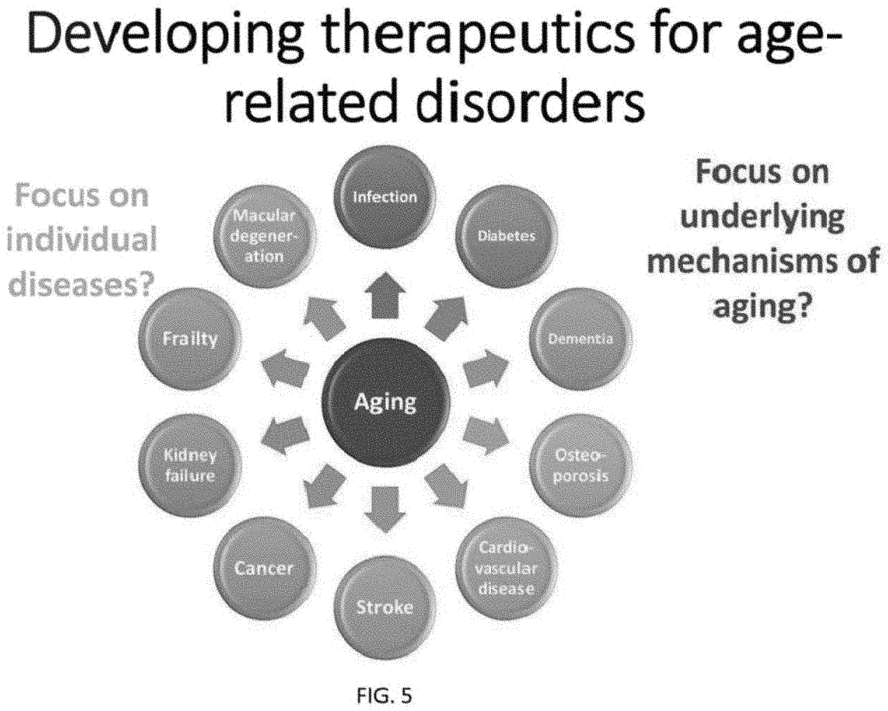

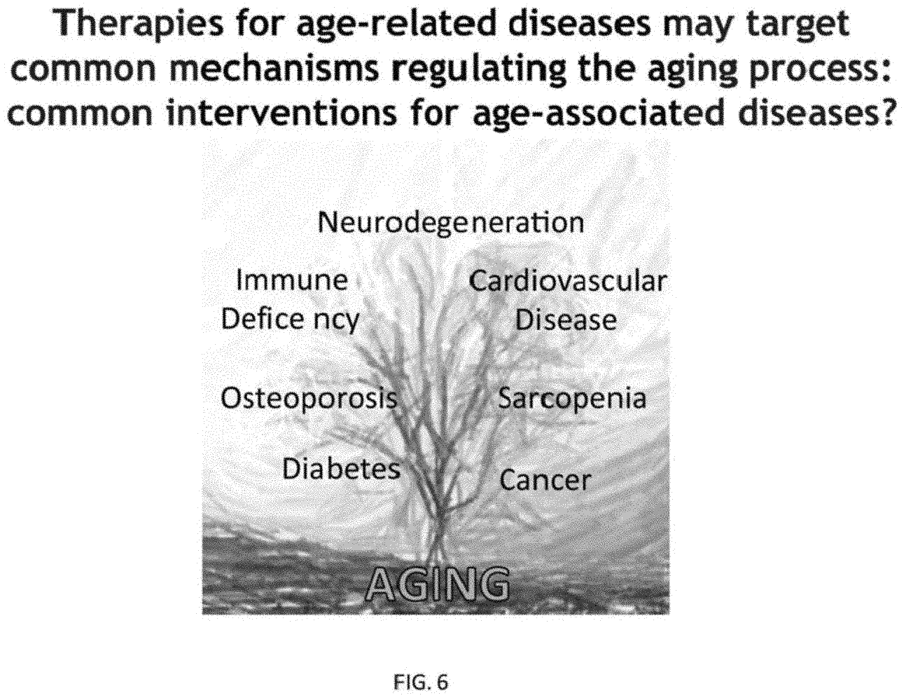

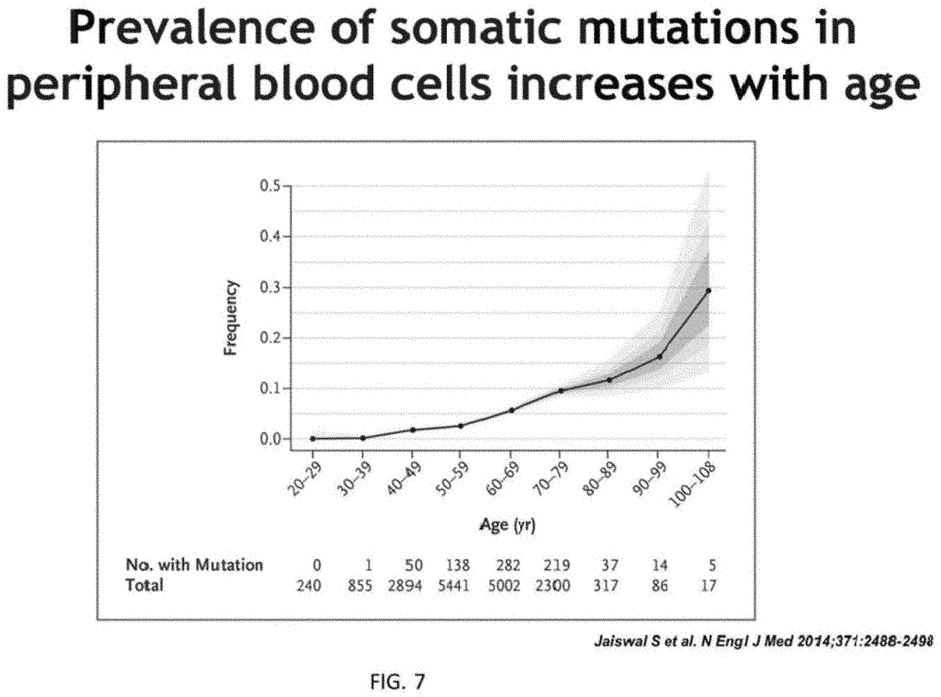

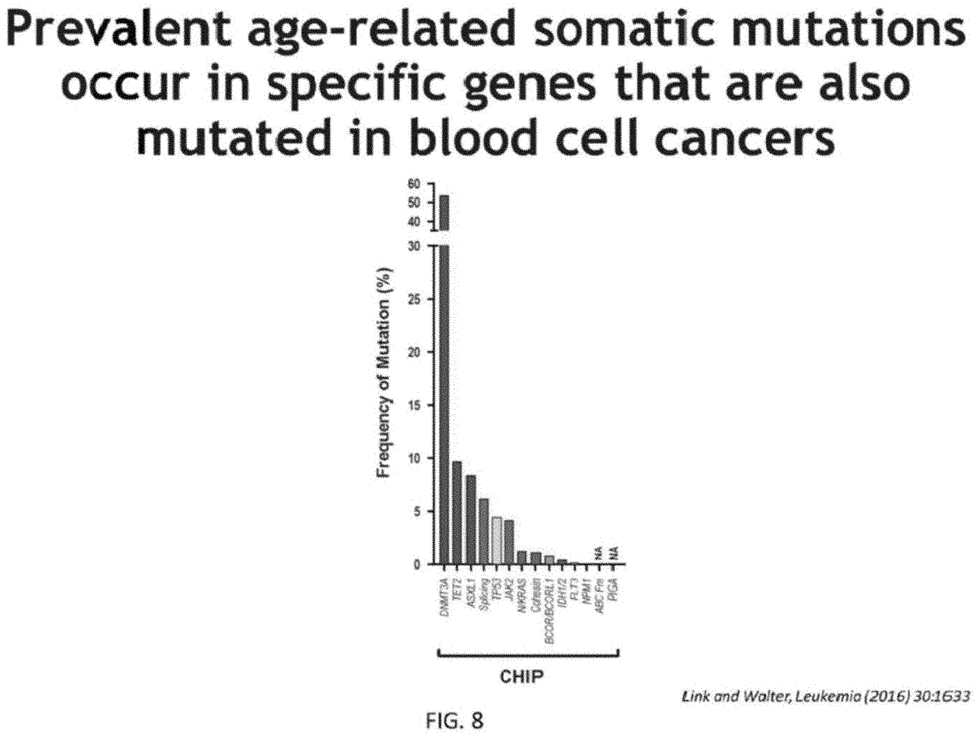

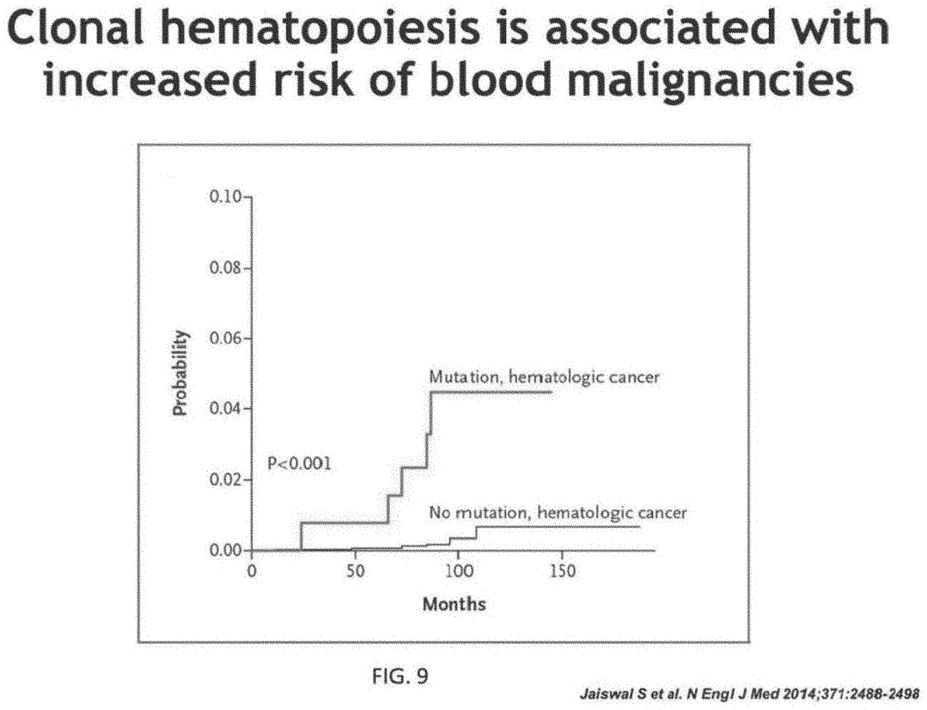

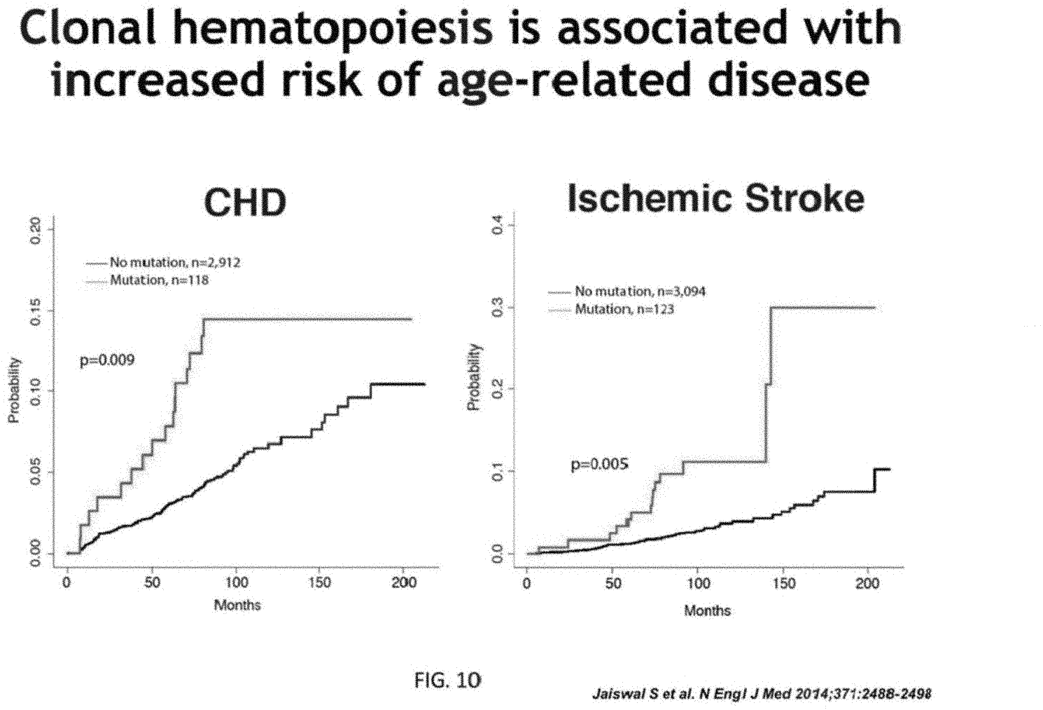

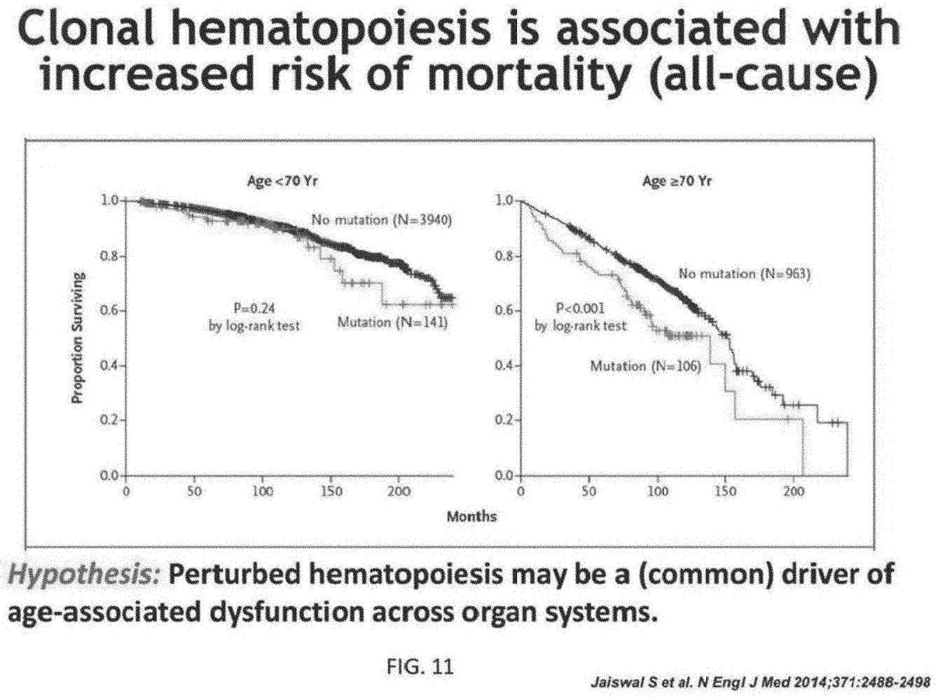

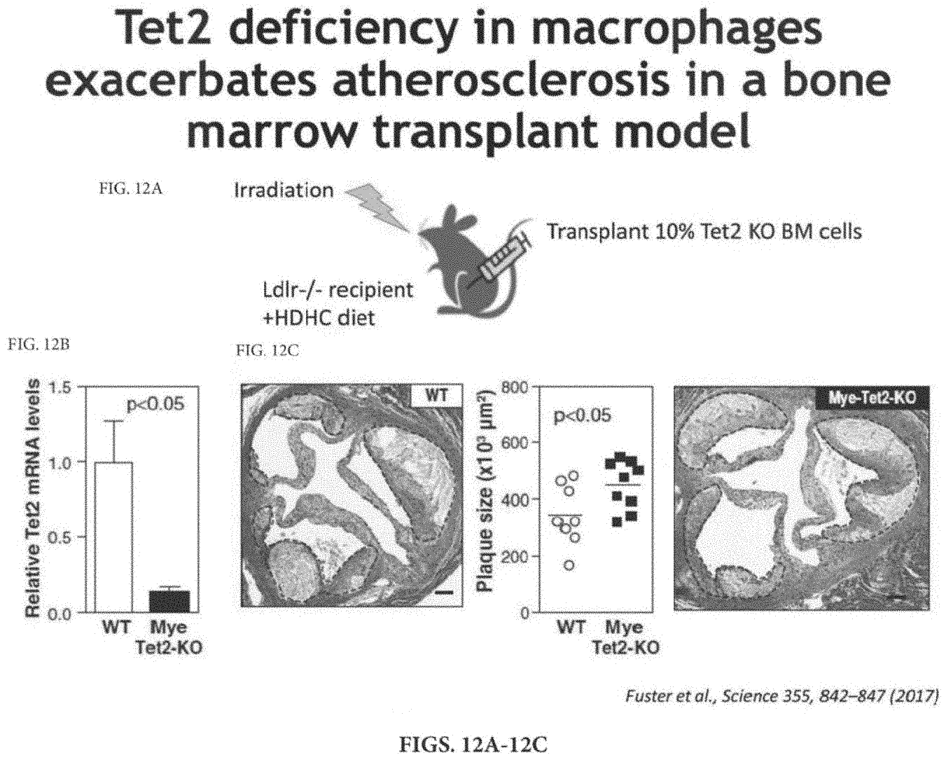

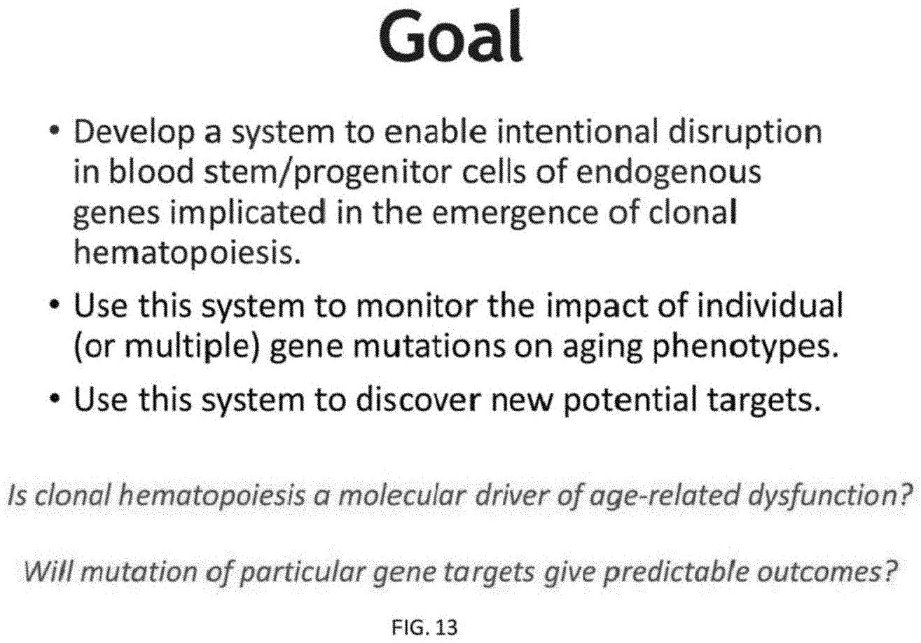

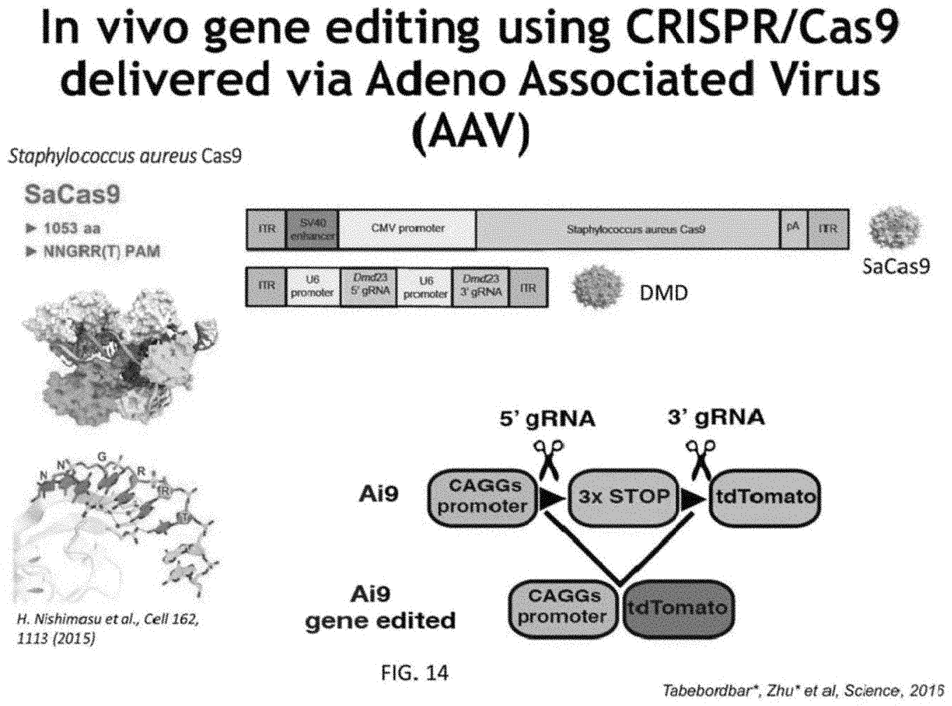

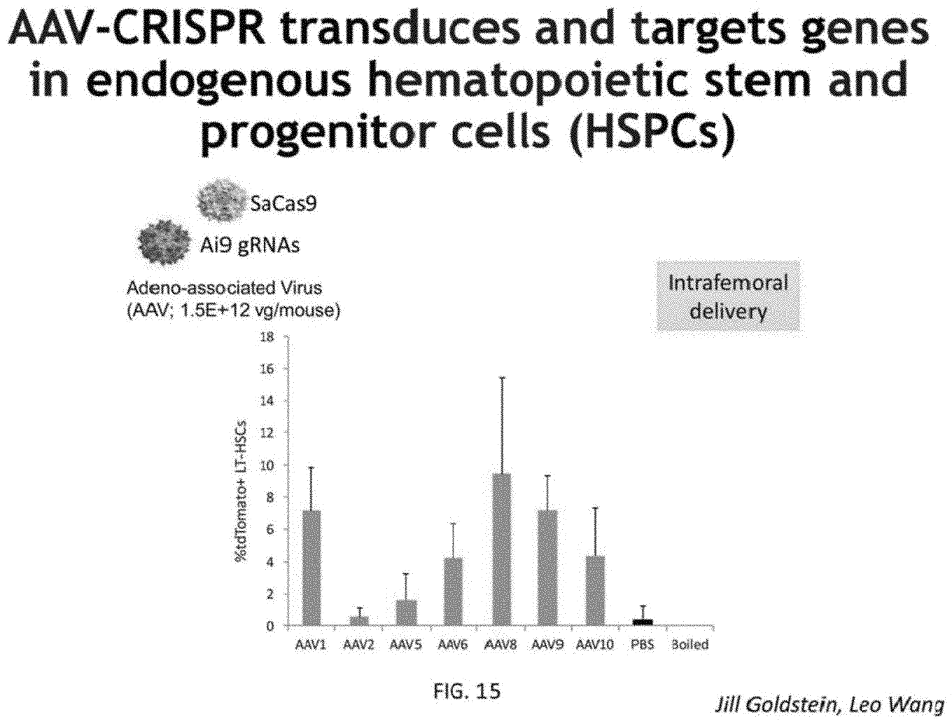

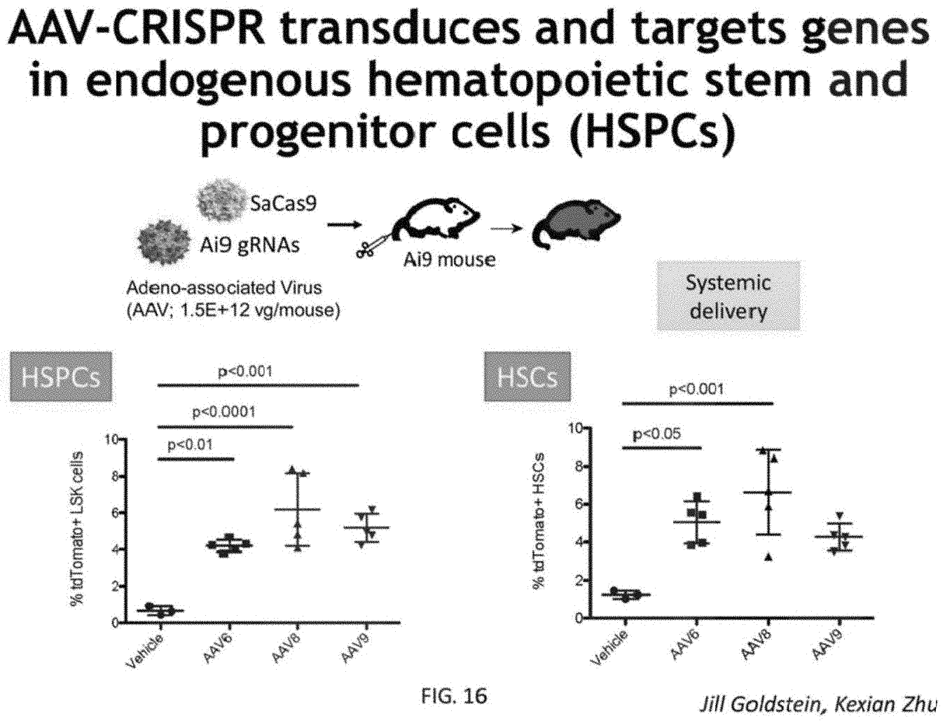

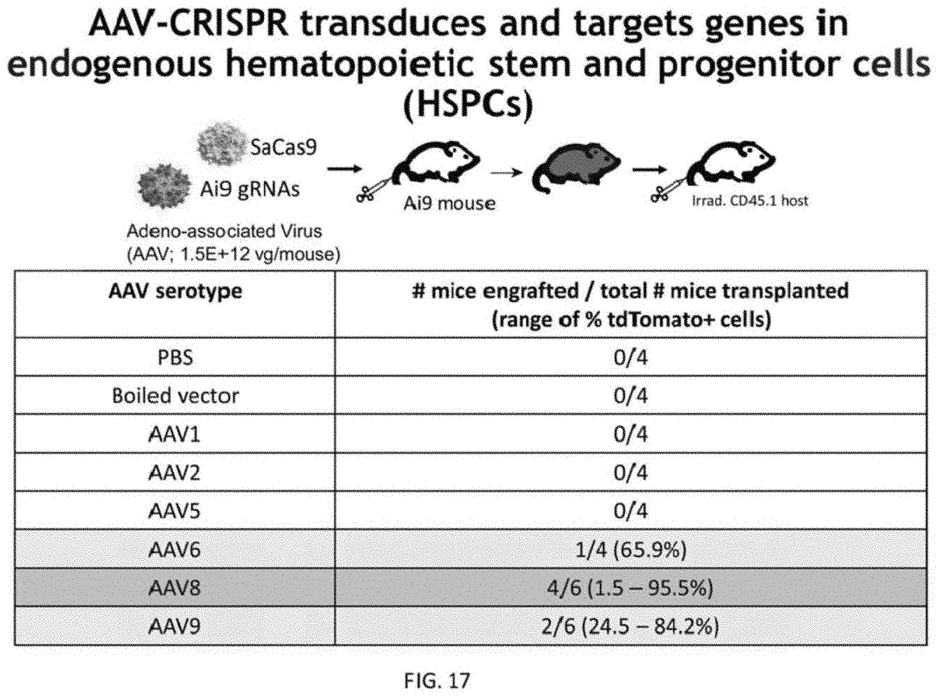

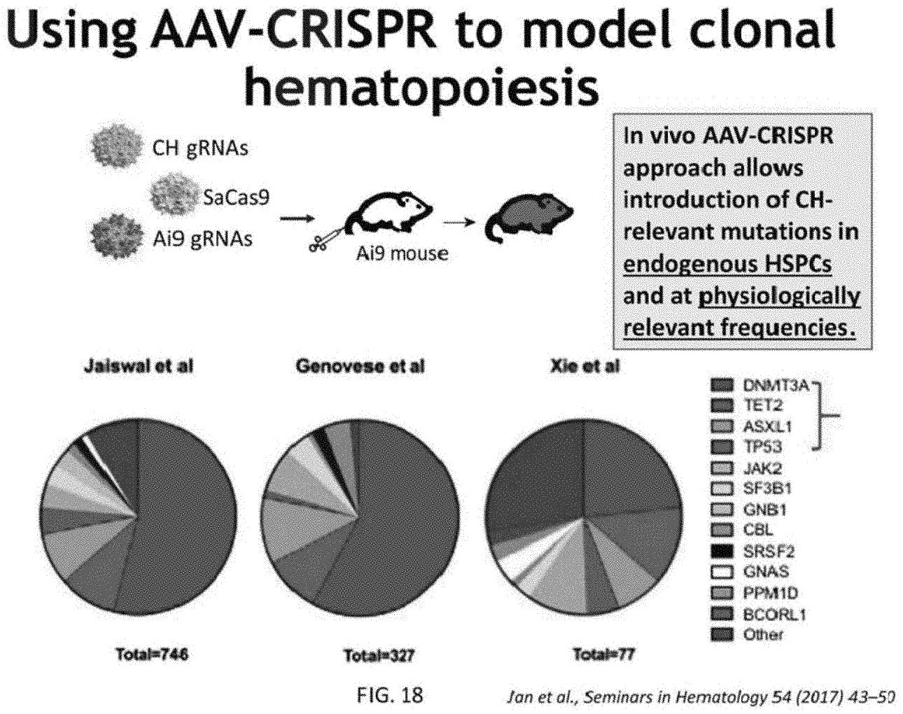

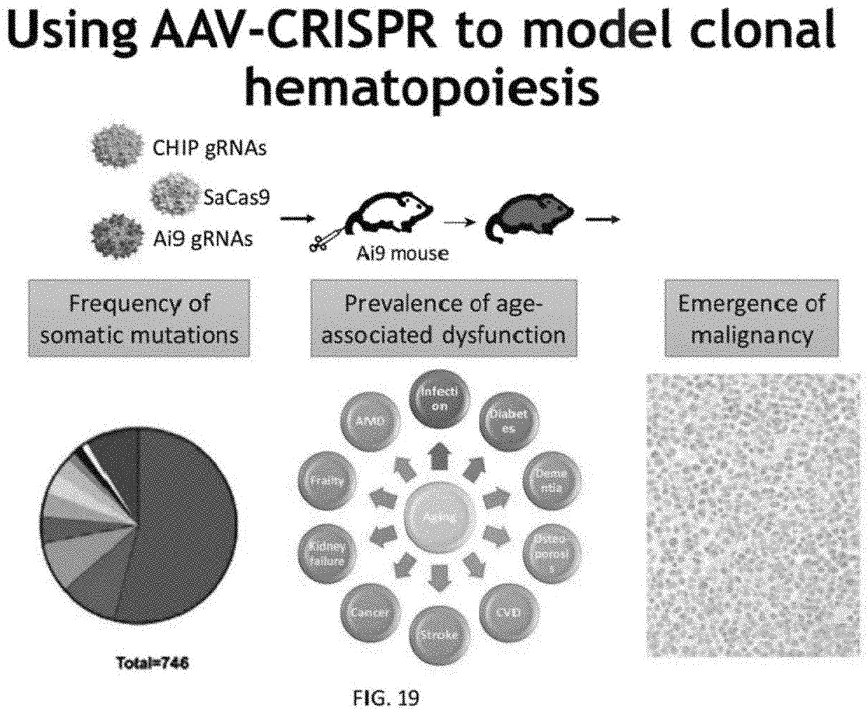

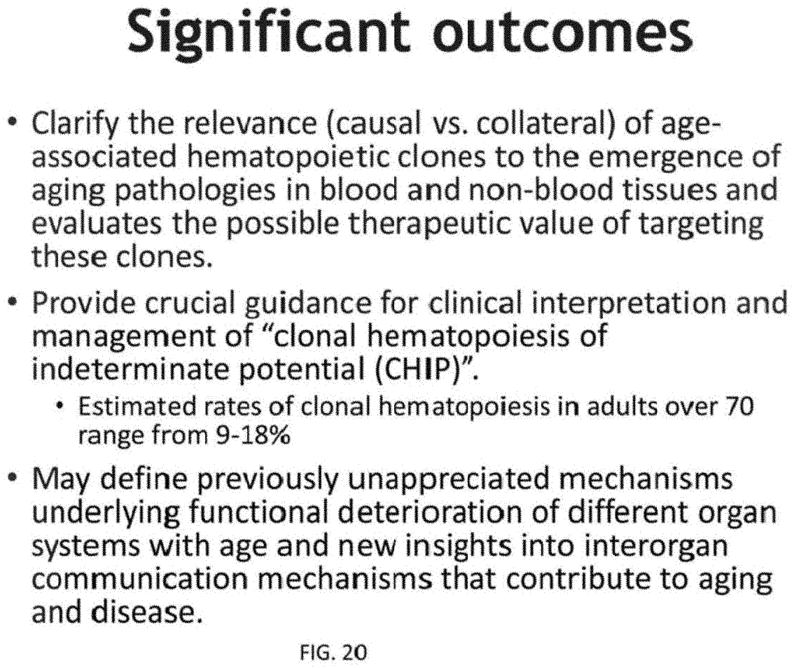

### REFERENCES

2. Tabebordbar et al, In vivo gene editing in dystrophic mouse muscle and muscle stem cells. Science. 2016.
3. Genovese, G., et al. Clonal hematopoiesis and blood-cancer risk inferred from blood DNA sequence. N Engl J Med 371, 2477-2487 (2014).
4. Jaiswal, S., et al. Age-related clonal hematopoiesis associated with adverse outcomes. N Engl J Med 371, 2488-2498 (2014).
5. Xie, M., et al. Age-related mutations associated with clonal hematopoietic expansion and malignancies. Nat Med 20, 1472-1478 (2014).

### DETAILED DESCRIPTION OF THE INVENTION

Disclosed is a strategy (i.e., method) for in vivo modification of DNA sequences within endogenous hematopoietic (blood-forming) stem and progenitor cells (HSPCs). This strategy utilizes viral (e.g., AAV-mediated) delivery of sequence targeting nucleases into blood lineage cells in vivo. AAVs can be injected directly into the bone marrow or delivered systemically. Proof-of-concept with this system has been demonstrated using AAV-mediated delivery of Cre recombinase into a fluorescent reporter mouse (the Ai9 reporter, which exhibits red fluorescence upon excision of sequences flanked by loxP Cre-recognition sites); however, the system can be easily adapted to deliver other relevant nucleases, including CRISPR-Cas9. We have shown editing of up to 9% of endogenous HSPCs by fluorescence activated cell sorting (FACS), and confirmed modification of the most primitive long-term reconstituting HSCs by transplantation assays. Further, disclosed herein is data showing AAV-CRISPR/Cas9 mediates disruption of an endogenous gene in the genome of endogenous hematopoietic stem cells. This system could be useful in a number of ways:

The methods of the invention can be used clinically to introduce therapeutic gene disruptions in endogenous HSPCs (e.g., for disruption of the Bcl11A erythroid enhancer, which would enable expression of fetal hemoglobin in adult blood cells as a therapeutic strategy for beta-hemoglobinopathies, or for disruption of the HIV co-receptor CCR5 for induction of blood cell resistance to HIV infection).

The methods of the invention can be combined with delivery of homologous donor templates to enable therapeutic gene replacement (e.g., to correct the sequence of disease causing mutations, such as the sickle variant of HBB, which causes sickle cell anemia, replacing these mutant sequences with normal ones).

The methods of the invention can be used experimentally to evaluate the role of specific gene products in blood cell function and blood disease (e.g., by introduction of mutations that have been identified in human patients but for which functional evaluation has not been done) to segregate causative from associative mutations. One example is to introduce mutations associated with clonal hematopoiesis in humans. The presence of these mutations has been associated with aging and with increased risk of malignancy, cardiovascular disease and stroke, but whether these changes are themselves causative of these pathologies (or mere biomarkers) has not been established. Recent human studies have shown that normal aging is associated with an increased frequency of somatic mutations in the hematopoietic system, which provide a competitive growth advantage to the mutant cell and allow its progressive clonal expansion (clonal hematopoiesis).

Gene correction in endogenous HSPCs, in particular, has the potential to overcome two key limitations faced by similar approaches that rely on HSPC isolation, ex vivo modification, and subsequent transplantation.

First, strong data indicate that the only cells capable of long-term hematopoietic reconstitution following transplant are the most primitive subset of hematopoietic stem cells (LT-HSCs); however, multiple recent studies indicate that the engraftment efficiency of these cells is reduced following ex vivo manipulation, leading to reduced representation of gene-modified cells in the reconstituted hematopoietic systems of transplant recipients (9, 12, 17). By accomplishing HSC editing in situ, our strategy avoids the need for transplantation, thereby circumventing this “engraftment problem”.

Second, recent data tracing the in vivo clonal dynamics during steady-state hematopoiesis has raised the possibility that the unique function of HSCs as hematopoietic regenerative units may be restricted to the transplant setting, and that endogenous hematopoiesis may be supported largely by a collection of very long-lived, lineage-restricted progenitor cells (25, 26). Given the higher rates of cell division observed for these progenitors, it is possible that they may be more amenable to HDR-based gene editing; however, since they fail to engraft long-term following transplantation (16), they have not been considered as viable targets for ex vivo gene editing approaches. Our strategy of in vivo editing could overcome this limitation by enabling the modification of such long-acting progenitor cells in situ, where their long-term activity is preserved, thereby providing an alternative or additional source of modified regenerative cells for therapy.

### REFERENCES

9. Hoban Correction of the sickle cell disease mutation in human hematopoietic stem/progenitor cells. Blood. 2015; 125(17):2597-604. doi: 10.1182/blood-2014-12-615948.
12. Genovese et al, Targeted genome editing in human repopulating haematopoietic stem cells. Nature. 2014; 510(7504):235-40.
16. Seita J, Weissman I L. Hematopoietic stem cell: self-renewal versus differentiation. Wiley interdisciplinary reviews Systems biology and medicine. 2010; 2(6):640-53.
17. Wang et al, Homology-driven genome editing in hematopoietic stem and progenitor cells using ZFN mRNA and AAV6 donors. Nature biotechnology. 2015; 33(12):1256-63.
25. Sun et al, Clonal dynamics of native haematopoiesis. Nature. 2014; 514(7522):322-7.
26. Busch et al, Fundamental properties of unperturbed haematopoiesis from stem cells in vivo. Nature. 2015; 518(7540):542-6.

In some aspects, the invention is directed towards a method for modifying the genome of Hematopoietic Stem and Progenitor Cells (HSPCs) in a subject (e.g., human, mouse), comprising contacting the subject with a virus (e.g., adeno-associated virus (AAV)), wherein the virus transduces a nucleic acid sequence encoding a sequence targeting nuclease into the HSPCs; and modifying the genome of the HSPCs with the sequence targeting nuclease. In some embodiments, at least about 0.1%, 0.5%, 1%, 2%, 3%, 5%, 10%, 15%, 20%, 25%, 30%, 35%, 40%, 45%, 50% or more of the HSPCs or a subset (e.g. LT-HSC) thereof are modified. In some embodiments, at least about 0.1%, 0.5%, 1%, 2%, 3%, 5%, 10%, 15%, 20%, 25%, 30%, 35%, 40%, 45%, 50% or more of the genome of the HSPCs or a subset (e.g. LT-HSC) thereof are modified via homologous recombination (e.g., a genomic sequence is replaced or inserted). In some embodiments, at least about 0.1%, 0.5%, 1%, 2%, 3%, 5%, 10%, 15%, 20%, 25%, 30%, 35%, 40%, 45%, 50% or more of the genome of the HSPCs or a subset (e.g. LT-HSC) thereof are modified via non-homologous end-joining (NEJ) (e.g., a genomic sequence is deleted).

Suitable viruses include, e.g., adenoviruses, adeno-associated viruses, retroviruses (e.g., lentiviruses), vaccinia virus and other poxviruses, herpesviruses (e.g., herpes simplex virus), and others. The virus may or may not contain sufficient viral genetic information for production of infectious virus when introduced into host cells, i.e., viral vectors may be replication-competent or replication-defective.

In some embodiments, the virus is adeno associated virus. Adeno-associated virus (AAV) is a small (20 nm) replication-defective, nonenveloped virus. The AAV genome a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA that inhibits ATPIF1, operably linked to a promoter, is inserted between the inverted terminal repeats (ITR) of the AAV genome. Adeno-associated viruses (AAV) and their use as vectors, e.g., for gene therapy, are also discussed in Snyder, R O and Moullier, P., Adeno-Associated Virus Methods and Protocols, Methods in Molecular Biology, Vol. 807. Humana Press, 2011.

In some embodiments, the AAV used in the inventive method is AAV serotype 6, 8 or 9. In some embodiments, the AAV serotype is AAV serotype 2. Any AAV serotype may be used as appropriate and is not limited.

Another suitable AAV may be, e.g., rhlO [WO 2003/042397]. Still other AAV sources may include, e.g., AAV9 [U.S. Pat. No. 7,906,111; US 2011-0236353-A1], and/or hu37 [see, e.g., U.S. Pat. No. 7,906,111; US 2011-0236353-A1], AAV 1, AAV2, AAV3, AAV4, AAV5, AAV6, AAV6.2, AAV7, AAV8, [U.S. Pat. Nos. 7,790,449; 7,282,199] and others. See, e.g., WO 2003/042397; WO 2005/033321, WO 2006/110689; U.S. Pat. Nos. 7,790,449; 7,282,199; 7,588,772B2 for sequences of these and other suitable AAV, as well as for methods for generating AAV vectors. Still other AAV may be selected, optionally taking into consideration tissue preferences of the selected AAV capsid. A recombinant AAV vector (AAV viral particle) may comprise, packaged within an AAV capsid, a nucleic acid molecule containing a 5 ‘ AAV ITR, the expression cassettes described herein and a 3’ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3 ‘ ITR. A shortened version of the 5’ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

A single-stranded AAV viral vector may be used. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, ULB, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of which is incorporated herein by reference in its entirety: U.S. Pat. Nos. 5,139,941; 5,741,683; 6,057, 152; 6,204,059; 6,268,213; 6,491,907; 6,660,514; 6,951,753; 7,094,604; 7, 172,893; 7,201,898; 7,229,823; and 7,439,065.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production.

The virus may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, the promoter is selected from CMV promoter and U6 promoter.

In some embodiments, the virus (e.g., AAV) is administered systemically (e.g., intravenously) or is injected into bone marrow. Alternatively, other routes of administration may be selected (e.g., oral, inhalation, intranasal, intratracheal, intraarterial, intraocular, intravenous, intramuscular, and other parental routes). The method of administration is not limited.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g. a human, non-human primate, rodent (e.g., mouse, rat, rabbit), ungulate (e.g., ovine, bovine, equine, caprine species), canine, or feline. In some embodiments, a human subject is between newborn and 6 months old. In some embodiments, a human subject is between 6 and 24 months old. In some embodiments, a human subject is between 2 and 6, 6 and 12, or 12 and 18 years old. In some embodiments a human subject is between 18 and 30, 30 and 50, 50 and 80, or greater than 80 years old. In some embodiments, the subject is at least about 50, 60, 65, 70, 75, 80, 85, or 90 years of age. In some embodiments, a subject is an adult. For purposes hereof a human at least 18 years of age is considered an adult. In some embodiments a subject is an embryo. In some embodiments a subject is a fetus. In certain embodiments an agent is administered to a pregnant female in order to treat or cause a biological effect on an embryo or fetus in utero.

There are currently four main types of sequence targeting nucleases (i.e., targetable nucleases, site specific nucleases) in use: zinc finger nucleases (ZFNs), transcription activator-like effector nucleases (TALENs), and RNA-guided nucleases (RGNs) such as the Cas proteins of the CRISPR/Cas Type II system, and engineered meganucleases. ZFNs and TALENs comprise the nuclease domain of the restriction enzyme FokI (or an engineered variant thereof) fused to a site-specific DNA binding domain (DBD) that is appropriately designed to target the protein to a selected DNA sequence. In the case of ZFNs, the DNA binding domain comprises a zinc finger DBD. In the case of TALENs, the site-specific DBD is designed based on the DNA recognition code employed by transcription activator-like effectors (TALEs), a family of site-specific DNA binding proteins found in plant-pathogenic bacteria such as Xanthomonas species. The Clustered Regularly Interspaced Short Palindromic Repeats (CRISPR) Type II system is a bacterial adaptive immune system that has been modified for use as an RNA-guided endonuclease technology for genome engineering. The bacterial system comprises two endogenous bacterial RNAs called crRNA and tracrRNA and a CRISPR-associated (Cas) nuclease, e.g., Cas9. The tracrRNA has partial complementarity to the crRNA and forms a complex with it. The Cas protein is guided to the target sequence by the crRNA/tracrRNA complex, which forms a RNA/DNA hybrid between the crRNA sequence and the complementary sequence in the target. For use in genome modification, the crRNA and tracrRNA components are often combined into a single chimeric guide RNA (sgRNA or gRNA) in which the targeting specificity of the crRNA and the properties of the tracrRNA are combined into a single transcript that localizes the Cas protein to the target sequence so that the Cas protein can cleave the DNA. The sgRNA often comprises an approximately 20 nucleotide guide sequence complementary or homologous to the desired target sequence followed by about 80 nt of hybrid crRNA/tracrRNA. One of ordinary skill in the art appreciates that the guide RNA need not be perfectly complementary or homologous to the target sequence. For example, in some embodiments it may have one or two mismatches. The genomic sequence which the gRNA hybridizes is typically flanked on one side by a Protospacer Adjacent Motif (PAM) sequence although one of ordinary skill in the art appreciates that certain Cas proteins may have a relaxed requirement for a PAM sequence. The PAM sequence is present in the genomic DNA but not in the sgRNA sequence. The Cas protein will be directed to any DNA sequence with the correct target sequence and PAM sequence. The PAM sequence varies depending on the species of bacteria from which the Cas protein was derived. Specific examples of Cas proteins include Cas1, Cas2, Cas3, Cas4, Cas5, Cash, Cas7, Cas8, Cas9 and Cas10. In some embodiments, the site specific nuclease comprises a Cas9 protein. For example, Cas9 from Streptococcus pyogenes (Sp), Neisseria meningitides, Staphylococcus aureus, Streptococcus thermophiles, or Treponema denticola may be used. The PAM sequences for these Cas9 proteins are NGG, NNNNGATT, NNAGAA, NAAAAC, respectively. In some embodiments, the Cas9 is from Staphylococcus aureus (saCas9).

A number of engineered variants of the site-specific nucleases have been developed and may be used in certain embodiments. For example, engineered variants of Cas9 and FokI are known in the art. Furthermore, it will be understood that a biologically active fragment or variant can be used. Other variations include the use of hybrid site specific nucleases. For example, in CRISPR RNA-guided FokI nucleases (RFNs) the FokI nuclease domain is fused to the amino-terminal end of a catalytically inactive Cas9 protein (dCas9) protein. RFNs act as dimers and utilize two guide RNAs (Tsai, Q S, et al., Nat Biotechnol. 2014; 32(6): 569-576). Site-specific nucleases that produce a single-stranded DNA break are also of use for genome editing. Such nucleases, sometimes termed “nickases” can be generated by introducing a mutation (e.g., an alanine substitution) at key catalytic residues in one of the two nuclease domains of a site specific nuclease that comprises two nuclease domains (such as ZFNs, TALENs, and Cas proteins). Examples of such mutations include D10A, N863A, and H840A in SpCas9 or at homologous positions in other Cas9 proteins. A nick can stimulate HDR at low efficiency in some cell types. Two nickases, targeted to a pair of sequences that are near each other and on opposite strands can create a single-stranded break on each strand (“double nicking”), effectively generating a DSB, which can optionally be repaired by HDR using a donor DNA template (Ran, F. A. et al. Cell 154, 1380-1389 (2013). In some embodiments, the Cas protein is a SpCas9 variant. In some embodiments, the SpCas9 variant is a R661A/Q695A/Q926A triple variant or a N497A/R661A/Q695A/Q926A quadruple variant. See Kleinstiver et al., “High-fidelity CRISPR-Cas9 nucleases with no detectable genome-wide off-target effects,” Nature, Vol. 529, pp. 490-495 (and supplementary materials)(2016); incorporated herein by reference in its entirety. In some embodiments, the Cas protein is C2c1, a class 2 type V-B CRISPR-Cas protein. See Yang et al., “PAM-Dependent Target DNA Recognition and Cleavage by C2c1 CRISPR-Cas Endonuclease,” Cell, Vol. 167, pp. 1814-1828 (2016); incorporated herein by reference in its entirety. In some embodiments, the Cas protein is one described in US 20160319260 “Engineered CRISPR-Cas9 nucleases with Altered PAM Specificity” incorporated herein by reference.

The nucleic acid encoding the sequence targeting nuclease should be sufficiently short to be included in the virus (e.g., AAV).

In some embodiments, the sequence targeting nuclease has at least about 80%, 85%, 90%, 91%, 92%, 93%, 94%, 95%, 96%, 97%, 98% or 99% polypeptide sequence identity to a naturally occurring targetable nuclease.

In some embodiments, the sequence targeting nuclease is a Zinc-Finger Nuclease (ZFN), a Transcription activator-like effector nuclease (TALEN), or a Cas9 nuclease.

In some embodiments, the method further comprises contacting the subject with a second virus (e.g., AAV) which transduces a nucleic acid sequence encoding one or more gRNAs. In some embodiment, the ratio of the first virus to the second virus is about 1:3 to about 1:100, inclusive of intervening ratios. For example, the ratio of the first virus to the second virus may be about 1:5 to about 1:50, or about 1:10, or about 1:20. Although not as preferred, the ratio may be 1:1 or there may be more second virus.

In some embodiments, the second virus encodes for two gRNAs that flank a genetic region of interest (e.g., a CHIP mutation, a mutation associated with a blood disorder). In some embodiments, the methods of the invention further comprise administration to the subject of homologous donor templates to enable therapeutic gene replacement (e.g., to correct the sequence of disease causing mutations, such as the sickle variant of HBB, which causes sickle cell anemia, replacing these mutant sequences with normal ones). Homologous recombination (HR) mediated repair (also termed homology-directed repair (HDR)) uses homologous donor DNA as a template to repair the break. If the sequence of the donor DNA differs from the genomic sequence, this process leads to the introduction of sequence changes into the genome.

In another embodiment, the method comprises a single AAV for delivery of gRNA and a second, different, Cas9-delivery system. For example, Cas9 (or Cpfl) delivery may be mediated by non-viral constructs, e.g., “naked DNA”, “naked plasmid DNA”, RNA, and mRNA; coupled with various delivery compositions and nanoparticles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, both of which are incorporated herein by reference.

In some embodiments, the method modifies the genome of CD34−, CD38−, SCA-1+, Thy1.1+/lo, C-kit+, lin−, CD135−, Slamf1/CD150+ hematopoietic stem cells (LT-HSCs). In some embodiments, the method modifies the genome of lineage restricted progenitor cells. In some embodiments, the method modifies a sufficient number and/or type of HSPCs to repopulate the subject's blood cells and treat hemoglobinopathies, Sickle cell disease (SCD), or β-thalassemia. In some embodiments, the method modifies a number of HSPC's to provide a physiologically accurate frequency (i.e., level) of somatic cells having CHIP mutations.

In some embodiments, the genome modification comprises the introduction or correction of a mutation associated with clonal hematopoiesis of indeterminate potential (CHIP). In some embodiments, the modification comprises the introduction or correction of a mutation associated with Sickle cell disease (SCD) or β-thalassemia.

In some embodiments, the method treats hemoglobinopathies, Sickle cell disease (SCD) or β-thalassemia. The effect of treatment may include reversing, alleviating, reducing severity of, delaying the onset of, curing, inhibiting the progression of, and/or reducing the likelihood of occurrence or recurrence of hemoglobinopathies, SCD or β-thalassemia or one or more symptoms or manifestations of hemoglobinopathies, SCD or β-thalassemia.

In some embodiments, the modification comprises correction of a mutation via homology-directed repair. In some embodiments, the modification activates or deactivates gene expression (e.g., expression of fetal hemoglobin).

In some embodiments, virus compositions can be formulated in dosage units to contain an amount of replication-defective virus that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

In some embodiments, the method further comprises assessing the fate or function of HSPC with genome modification. In some embodiments, the assessment comprises determining if the modification enhances self-renewal of HSPC. In some embodiments, the assessment comprises determining if the modification degrades self-renewal of HSPC. In some embodiments, multiple geneomic modifications are made to the HSPC with genome modification. In some embodiments, the genome modification comprises modification of one or more genes associated with biological processes. In some embodiments, the biological processes comprise epigenetic regulation or proteostasis (e.g., autophagy, ubiquitin-proteasome, heat shock response, anti-oxidant response, unfolded protein response).

In some embodiments, the second virus also transduces nucleic acid sequences encoding one or more gRNAs to a cell surface expressed molecule whose loss is non-pathogenic. Disruption of the a cell surface expressed molecule can be used as a marker indicating probable successful targeting of the genetic region of interest as well, since disruption of the cell surface expressed marker requires transduction of the virus having the sequence targeting nuclease and the second virus transducing the gRNAs to a genetic region of interest. The level of cell surface expressed marker on cells should be HIGH in cells containing 2 intact copies of the gene encoding it, LOW in cells containing 1 intact copy and 1 disrupted copy, and ABSENT in cells containing 2 disrupted copies. In some embodiments, the methods of the invention further comprise detection of the level of modification of the genetic region of interest (e.g., one or two alleles). In some embodiments, detection is accomplished by flow cytometry using an antibody specific to cell surface expressed marker.

Some aspects of the invention are directed to a method for in vivo modifying a genetic region of interest in a cell in a subject, comprising contacting the subject with a virus, wherein the virus transduces a nucleic acid sequence encoding a Cas9 nuclease into the cell; contacting the subject with a second virus which transduces a nucleic acid sequence encoding a first set of one or more gRNAs targeting the genetic region of interest and a second set of one or more gRNAs targeting a genetic region encoding or controlling the expression of a cell surface marker; modifying the genetic region of interest with the Cas9 nuclease; and modulating expression of the cell surface marker.

In some embodiments, loss and/or gain of the cell surface marker by the cell is non-pathogenic. In some embodiments, modulating the level of the cell surface marker is non-pathogenic. In some embodiments, the method further comprises detecting the likelihood or degree of modification of the genetic region of interest by detecting a change in the expression of the cell surface marker as compared to a control cell. In some embodiments, a change in the change in the expression of the cell surface marker is detected by immunochemistry (e.g., FACS). In some embodiments, the degree of modulation of the expression of the cell surface marker indicates whether one or both copies of a genetic region of interest are modified by the Cas9 nuclease. In some embodiments, the absence of expression of the cell surface marker indicates that both copies of a genetic region of interest are, or are likely to be, modified by the Cas9 nuclease. In some embodiments, the reduction of expression of the cell surface marker indicates that one copy of a genetic region of interest is, or is likely to be, modified by the Cas9 nuclease. In some embodiments, the high of expression of the cell surface marker indicates that both copies of a genetic region of interest are not, or are likely not, modified by the Cas9 nuclease.

The cell surface marker (e.g., non-pathogenic cell surface marker) is not limited and can be routinely determined in the art. In some embodiments, the cell surface marker is CCR5. The cell to be modified is not limited and can be any suitable cell in the art or described herein. In some embodiments, the cell is an HSPC.

Some aspects of the invention are directed to a method of screening for genetic regions coding for regulators of hematopoietic stem cell (HSC) self-renewal and/or differentiation, comprising contacting an HSC in vivo with a virus, wherein the virus transduces a nucleic acid sequence encoding a sequence targeting nuclease into the HSC; modifying a genetic region of the HSC with the sequence targeting nuclease; assessing the self-renewal and/or differentiation of the modified HSC; wherein if modification of the genetic region modulates self-renewal and/or differentiation of the HSC then the genetic region is identified as coding for a regulator of hematopoietic stem cell (HSC) self-renewal and/or differentiation. In some embodiments, the genetic region is a gene linked to dysregulated hematopoiesis and/or hematopoietic malignancy, or is linked to variations in HSC self-renewal activity. I

In some embodiments, the virus is adeno-associated virus (AAV). In some embodiments, the AAV is AAV serotype 6, 8, 9 or 10. The virus may be any suitable virus or virus described herein and is not limited.

In some embodiments, the virus is administered intravenously or is injected into bone marrow. The virus may be administered by any suitable method and is not limited. The virus may be administered by any method described herein.

In some embodiments, the sequence targeting nuclease is a Zinc-Finger Nuclease (ZFN), a Transcription activator-like effector nuclease (TALEN), or a Cas9 nuclease. The sequence targeting nuclease may be any nuclease described herein and is not limited.

In some embodiments, the methods further comprise contacting the subject with a second virus which transduces a nucleic acid sequence encoding one or more gRNAs, wherein the one or more gRNA target the genetic region. In some embodiments, the second virus is an AAV. The virus is not limited and may be any suitable virus described herein or in the art.

Some aspects of the invention are directed to a composition for modifying the genome of Hematopoietic Stem and Progenitor Cells (HSPCs) in a subject comprising a virus encoding a sequence targeting nuclease (e.g., targetable nuclease) as described herein.

Some aspects of the invention are directed to a composition for modifying the genome of Hematopoietic Stem and Progenitor Cells (HSPCs) in a subject comprising a virus encoding a sequence targeting nuclease (e.g., Cas9) as described herein and a second virus encoding one or gRNAs as described herein.

Some aspects of the invention are directed to a composition for modifying a genetic region in vivo comprising a virus encoding a sequence targeting nuclease (e.g., Cas9) as described herein and a second virus encoding one or gRNAs targeting the genetic region and one or more gRNAs targeting expression of a cell surface molecule as described herein.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

As used herein “A and/or B”, where A and B are different claim terms, generally means at least one of A, B, or both A and B. For example, one sequence which is complementary to and/or hybridizes to another sequence includes (i) one sequence which is complementary to the other sequence even though the one sequence may not necessarily hybridize to the other sequence under all conditions, (ii) one sequence which hybridizes to the other sequence even if the one sequence is not perfectly complementary to the other sequence, and (iii) sequences which are both complementary to and hybridize to the other sequence.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### EXAMPLES

### Studies of Clonal Hematopoiesis

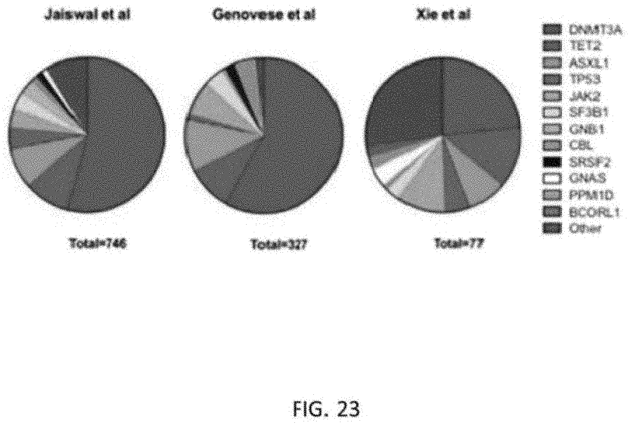

In this project, we test the possibility that perturbed hematopoiesis (blood formation) may be a common driver of age-associated dysfunction across organ systems by applying a novel in vivo gene editing system to introduce specific somatic mutations that are frequently associated with clonal hematopoiesis in aging humans (particularly Dnmt3a, Tet2 and Asxl1, see FIG. 23) into a subset of mature blood cells and blood progenitors in young and middle-aged mice. We will then monitor the rate of emergence of well-characterized, age-associated pathologies in three different non-hematopoietic organ systems (the skeletal muscle, brain and heart) that show profound and well-defined alterations with advancing age and for which prior studies indicate an increased likelihood of disease in individuals with clonally expanded mutant blood cells (4). In particular, we will monitor for the premature emergence of cardiac hypertrophy and atherosclerotic plaques, loss of skeletal muscle mass and regenerative potential, and perturbation of the cerebral vasculature with loss of neural stem, using the in vivo, physiological and histological methods that our team has successfully applied in previously published studies of aging biology (6-8).

This work will answer a critical and timely question in aging biology and gerontology—is the emergence of clonal mutations in the blood system a common driver of aging pathology in non-blood organs? It will further provide crucial guidance for the clinical interpretation and management of individuals identified to harbor this condition (recently termed clonal hematopoiesis of indeterminate potential (CHIP)). Such guidance is currently lacking (9) and urgently needed, as it is estimated that rates of clonal hematopoiesis in individuals over the age of 70 can range from 9-18% (3-5,10), and it is entirely unclear at present whether CHIP represents a causal or collateral aspect of aging effects in non-blood organs.

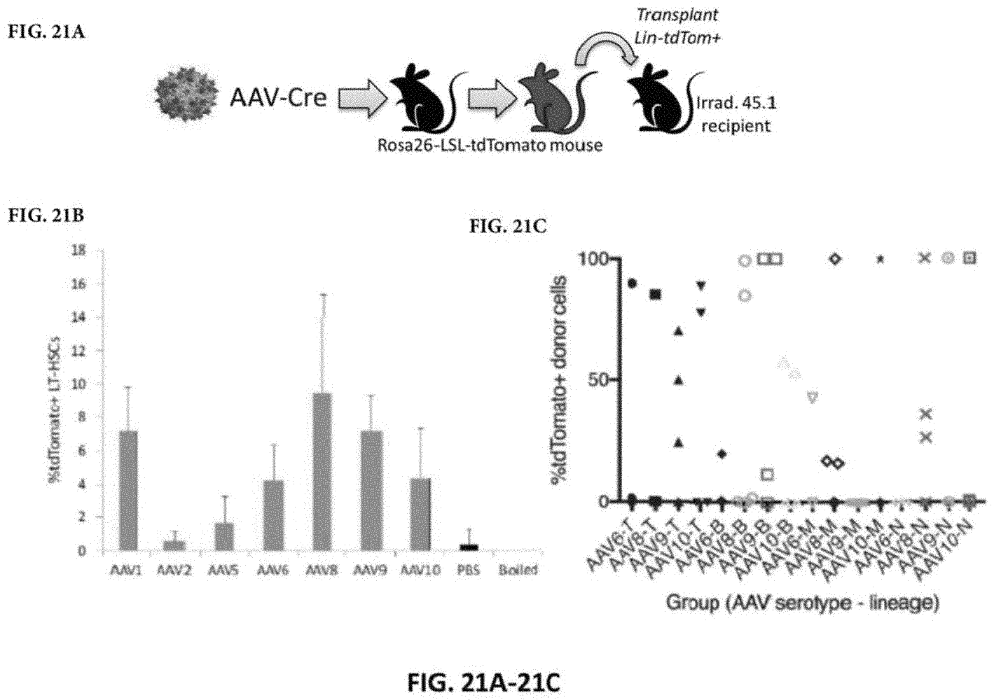

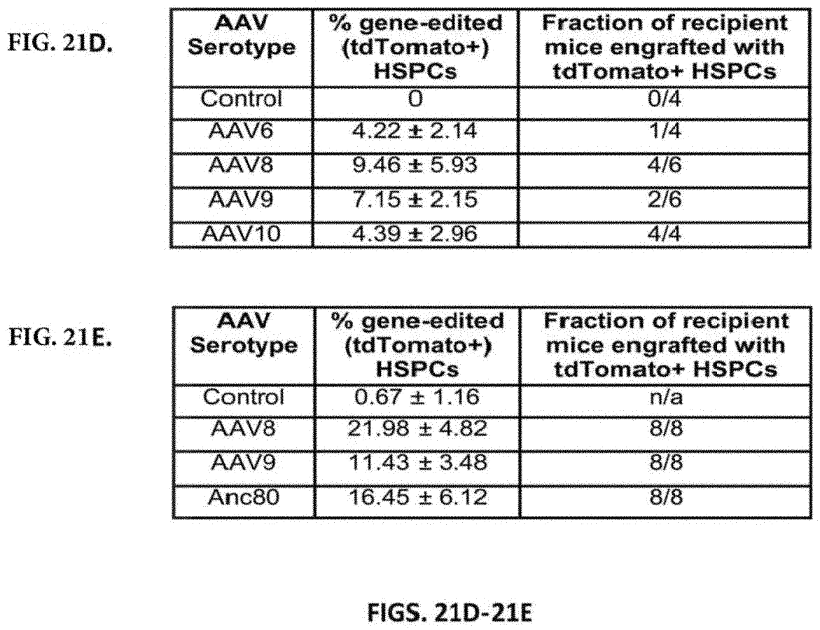

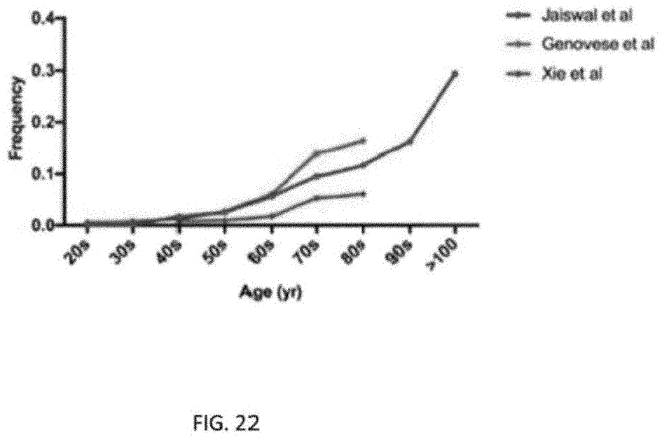

Our approach builds on the powerful tools and unique expertise we have developed over the past 13 years in hematopoiesis, in vivo stem cell modification and aging physiology. With a single exception (11), studies to date of clonal hematopoiesis have been limited to correlative assessments. In addition, the one recently published study that sought to evaluate a possible causal linkage between CHIP and non-blood organ dysfunction tested the effect of only a single somatic mutation (in Tet2) and used a much less physiologically relevant system, applying total body irradiation in LDL receptor null mice followed by bone marrow transplantation with relatively large numbers of mutant cells (11). In contrast, our approach will use genetically normal animals in which human-relevant mutations in individual genes or combinations of genes will be introduced at physiologically relevant low frequencies via in vivo CRISPR/Cas9-mediated gene editing. As documented in our published (12) and preliminary studies (see FIG. 21), this lab has developed and optimized a novel methodology through which Cas9, together with single or multiplexed genome-targeting guide RNAs, can be delivered systemically using adenoassociated viral (AAV) vectors to modify endogenous hematopoietic progenitors and mature cells.

### BIBLIOGRAPHY

1. Wagers, A. J. Aging stem cells and their niches: possibilities for regenerative medicine. Experimental Medicine (YOSHIDA, TOKYO) 31, 3348-3353 (2013).
2. Genovese, G., Jaiswal, S., Ebert, B. L. & McCarroll, S. A. Clonal hematopoiesis and blood-cancer risk. N Engl J Med 372, 1071-1072 (2015).
3. Genovese, G., et al. Clonal hematopoiesis and blood-cancer risk inferred from blood DNA sequence. N Engl J Med 371, 2477-2487 (2014).
4. Jaiswal, S., et al. Age-related clonal hematopoiesis associated with adverse outcomes. N Engl J Med 371, 2488-2498 (2014).
5. Xie, M., et al. Age-related mutations associated with clonal hematopoietic expansion and malignancies. Nat Med 20, 1472-1478 (2014).
6. Sinha, M., et al. Restoring systemic GDF11 levels reverses age-related dysfunction in mouse skeletal muscle. Science 344, 649-652 (2014).
7. Loffredo, F. S., et al. Growth differentiation factor 11 is a circulating factor that reverses age-related cardiac hypertrophy. Cell 153, 828-839 (2013).
8. Katsimpardi, L., et al. Vascular and neurogenic rejuvenation of the aging mouse brain by young systemic factors. Science 344, 630-634 (2014).
9. Heuser, M., Thol, F. & Ganser, A. Clonal Hematopoiesis of Indeterminate Potential. Dtsch Arztebl Int 113, 317-322 (2016).
10. Jan, M., Ebert, B. L. & Jaiswal, S. Clonal hematopoiesis. Semin Hematol 54, 43-50 (2017).
11. Fuster, J. J., et al. Clonal hematopoiesis associated with TET2 deficiency accelerates atherosclerosis development in mice. Science 355, 842-847 (2017).
12. Tabebordbar, M., et al. In vivo gene editing in dystrophic mouse muscle and muscle stem cells. Science 351, 407-411 (2016).

Treatment of SCD and β-Thalassemia

Sickle cell disease (SCD) and β-thalassemia are autosomal recessive diseases that affect hundreds of thousands of patients in the United States and millions of patients worldwide. Both diseases are caused by mutations in the β-globin gene. SCD is caused by a single point mutation that results in a glutamic acid to valine change at position 6 of the β-globin protein, whereas β-thalassemia can result from any of a number of mutations throughout the β-globin gene that cause decreased β-globin protein expression. Currently, the only curative therapy for SCD or β-thalassemia is allogeneic hematopoietic stem cell transplantation (HSCT). Yet, while allogeneic HSCT is successful in >90% of patients who are completely healthy and have a well-matched sibling donor, success rates for patients with unrelated donors or patients with end-organ damage or iron overload are significantly lower (6, 7).

Gene therapy represents an alternative to allogeneic HSCT whereby modified autologous hematopoietic stem cells (HSCs) would be transplanted back into the patient in order to cure the disease. There are currently multiple clinical trials either under way or about to start in which a lentivirus will be used to deliver a functional β-globin gene to HSCs, and these modified HSCs are then transplanted back into the patient. While available data suggest that lentiviral modification may carry a lower risk of insertional oncogenesis than gamma-retroviral modification, the safety of lentiviral vectors has not been completely confirmed in clinical trials. In fact, the single published β-thalassemia patient who was treated with lentiviral gene therapy developed clonal, though currently non-malignant, erythropoiesis from the activation of a genomic proto-oncogene (8), raising concerns about the potential for subsequent malignant transformation.

As an alternative gene therapy strategy, we and others (9, 10) have been working to develop genome editing approaches, based on homology directed recombination (HDR) to replace the mutated DNA at the β-globin locus. Instead of integrating an additional copy of the β-globin gene, this strategy aims to functionally correct at least one of the two mutant genes, converting homozygous mutant cells to heterozygous or homozygous functional cells by the replacing one or both of the mutant alleles with a cDNA encoding a full-length and fully functional β-globin. Importantly, this single targeting strategy would be applicable for therapy in a broad spectrum of SCD and β-thalessemia patients, for the most part independent of the precise mutation site, and could be used to convert SCD or β-thalassemia into sickle trait or β-thalassemia trait.

Towards that end, we have worked to engineer zinc finger nucleases (ZFNs), TAL effector nucleases (TALENs), and RNA-guided nucleases (RGENs, of the CRISPR/Cas9 class), to target near the SCD mutation site in exon 1 of the human β-globin gene. Work in our lab and others has demonstrated in cell lines and primary human CD34+ hematopoietic stem and progenitor cells (HSPCs) that such nucleases support HR-mediated modification at the human β-globin locus (HBB) in up to 20% of cells (1, 9-11), leading to production of normal hemoglobin tetramers (1, 9) that can substantially reduce sickling of SCD red blood cells (10). Yet, in our studies and those of others, preservation of hematopoietic engraftment potential after ex vivo gene editing has proved extremely challenging (9, 12). Indeed, recent publications have reported a substantial reduction (5-50-fold) in mean levels of genome modification at the β-globin locus after transplantation of gene-edited CD34+ cells into immunodeficient mice, with large numbers of cells required (1) and several recipient mice lacking any evidence of persistence of β-globin targeted cells (9). Similar concerns about potential loss of modified cells after gene transfer and transplant have been raised in recent discussions about Bluebird Bio's ongoing Lentiglobin trials (ASH Abstract #201, Dec. 6, 2015). Thus, while strong evidence supports the feasibility of phenotypic rescue via gene replacement in HSPCs as a therapeutic strategy for SCD and β-thalassemia, current limitations for preserving the hematopoietic reconstituting abilities of these cells after ex vivo culture presents a significant challenge for future clinical application, and argues for a radically different approach to overcome this “engraftment problem”.

With this in mind, we aim to develop novel in vivo gene editing strategies for correction of disease-causing β-globin mutations. Our work will test the hypothesis that β-globin gene modification in HSPCs can be achieved in situ, without the need for HSPC harvest, purification, culture or re-implantation. This hypothesis is supported by recent work in my lab that clearly documents therapeutic gene editing of the Dmd locus in endogenous muscle stem cells after delivery of the genome editing machinery via adeno-associated virus (AAV) (2), and our preliminary data demonstrating its further applicability for HSPC modification. The experiments proposed here will take a systematic approach, building on the extensive knowledge and resources we have generated through our prior studies, to test a single, central Specific Aim: To test the hypothesis that AAV-mediated delivery of gene editing components in vivo can enable targeted modification of endogenous HSPCs at the β-globin locus and rescue disease phenotypes in mutant cells.

Towards Curative Therapies for β-Hemoglobinopathies.

Currently, the only curative therapy for SCD or β-thalassemia is allogeneic hematopoietic stem cell transplantation (HSCT), in which a patient's own blood-forming system is replaced by donor cells from an individual with an unaffected HBB gene (6). However, while allogeneic HSCT is successful in >90% of patients who are healthy and have a well matched sibling donor, allogeneic HSCT is inaccessible for many patients due to a lack of appropriate immunologically matched donors, and success rates for patients with alternative donors or patients with end-organ damage and iron overload are significantly lower (6, 7). In addition, even for patients with well-matched donors, allogeneic HSCT carries with it substantial risks, including a significant risk for development of graft-versus-host disease (GVHD), in which a donor immune response against host cells causes widespread tissue inflammation and damage; graft failure, in which the transplanted cells fail to effectively re-establish hematopoietic cell production; or rejection, in which transplanted cells are destroyed by residual host immune cells. These considerable challenges have limited the widespread application of allogeneic HSCT to β-hemoglobinopathies.

A possible alternative strategy to allogeneic HSCT for the β-hemoglobinopathies has been brought to the fore by recent advances in the field of genome editing. “Genome editing” describes a scientific approach in which experimentally engineered programmable nucleases are used to insert, replace or remove segments of DNA within the genome of a living cell or organism (15). In the case of β-hemoglobinopathies, genome editing presents the possibility, explored recently xenograft systems (1, 9), of altering the mutant HBB sequences in a patient's own blood-forming cells and then returning these ‘corrected’ cells back to this same patient to support ongoing blood production. This strategy has significant advantages when compared to classical allogeneic HSCT in that (1) every patient can serve as his/her own donor, obviating the need for appropriately matched donors and overcoming immunological barriers to transplantation and GVHD triggers, and (2) editing strategies can be designed that replace the mutant gene with a full length, corrected HBB cDNA, allowing a common targeting strategy to be applied across the spectrum of HBB mutations underlying SCD and β-thalassemia. Importantly, because both SCD and β-thalassemia exhibit autosomal recessive inheritance, only one of the two mutant alleles must be corrected, as individuals carrying at least one unaffected allele typically do not display pathological symptoms.

Yet, a critical consideration for applying ex vivo genome editing strategies in SCD, β-thalassemia and other hematological diseases is the capacity to achieve modification in precursor cells that will support long-term replenishment of gene-modified cells upon transplantation. Such cells classically include the most primitive long-term hematopoietic stem cells (LT-HSCs), which are the only cells able to regenerate the entire blood system for the lifetime of the transplanted recipient (16). Yet published literature to date reveals significant challenges for retaining robust in vivo engraftment capacity following ex vivo gene editing in HSPCs. For example, an initial study using electroporation of IL2RG directed ZFNs, together with integrase-defective lentiviral vector (IDLV) encoded donor template to enable homology directed recombination (HDR), reported gene targeting rates of 5-12% in cultured human HSPCs, but saw a substantially lower fraction of edited human cells upon analysis of immunodeficient mice transplanted with these HSPCs (12). A second study, which likewise used an DLV encoded donor template or DNA oligonucleotide, together with β-globin directed ZFNs, reported an ˜50-fold reduction in mean levels of genome modification at the β-globin locus after transplant, with several recipient mice lacking any evidence of persistence of β-globin targeted cells (9). Finally, while recent studies (1) have achieved improved ex vivo HSPC transduction rates of up to 26-43%, using an AAV6 encoded donor template together with electroporation of mRNA encoded ZFNs (17) or Cas9 ribonucleoproteins (1), these groups still saw lower levels of editing in the most primitive CD34+CD133+CD90+ subset of human HSCs (17) and a reduced representation of gene-modified cells in transplanted mice (1, 17). Thus, while these important papers demonstrate key principles supporting the promise of therapeutic genome editing in HSPCs, including the capacity of primitive HSPCs to undergo homology directed repair (HDR) for gene insertion, the utility of AAVs as delivery vehicles for donor homology templates, and the fact that engraftment function can be at least partially preserved in gene edited cells, they also highlight the critical challenge of obtaining sufficient numbers of appropriately modified, engraftable cells via this route to support therapeutic blood reconstitution in patients.

Disclosed is a novel strategy to overcome this “engraftment problem”, by enabling in vivo gene editing in endogenous HSPCs. This work will provide novel, proof-of-concept data supporting a broadly applicable, and potentially curative, therapy for human β-hemoglobinopathies. Our pre-clinical testing strategy will be to replace mutated β-globin alleles in endogenous hematopoietic stem and progenitor cells (HSPCs) of SCD model mice (which express the human genes encoding HbA and HbS (18), with an intact β-globin cDNA that is resistant to polymerization (i.e., ‘anti-sickling’ β-globin (19-22)). We hypothesize that this approach will populate the blood-forming system with gene-edited daughter cells that produce sufficient amounts of functional, non-sickling β-globin to significantly ameliorate or even abrogate disease symptoms. Our innovative approach is based on our extensive and compelling preliminary data, outlined below, which demonstrate (1) that our existing nuclease and donor template systems can achieve detectable gene targeting and integration of an anti-sickling β-globin cDNA into the human HBB locus, (2) that this anti-sickling β-globin is appropriately induced upon erythroid differentiation of genome edited HSPCs, and (3) that endogenous tissue stem cells can be transduced by AAV in vivo to deliver functional genome editing machinery of relevance to human disease. Our experimental goals will therefore be to identify optimal conditions for in vivo HSPC transduction with genome modifying nucleases, adapt this system for site-directed HDR at the β-globin locus, and then test the therapeutic utility of this approach using disease-relevant SCD model mice.

Scientific Premise.

Current treatments for SCD and β-thalassemia are largely limited to disease-modifying therapies such as hydroxyurea and chronic transfusion. To develop potentially curative therapy for β-hemoglobinopathies, some investigators have pursued allogenic HSCT or ex vivo gene therapy in autologous HSPCs using lentiviral vectors that randomly integrate into the genome or that specifically modify the genome at the HBB locus to deliver an anti-sickling gene (6, 8) or correct the sickle mutation (9). As an alternative strategy, we propose herein a distinct genome editing approach based on homology directed recombination (HDR) in endogenous HSPCs. This strategy makes use of “designer” nucleases that can create a DNA double-strand break (DSB) at a specific sequence in exon 1 of HBB. Repair of this DSB by non-homologous end-joining (NHEJ) leads to insertions or deletions (indels) of small fragments of DNA at the site of the break; however, if the introduced DSB is repaired by HDR, using a DNA template (the ‘donor template’) that is provided in concert with the nuclease, then precise nucleotide changes, encoded in the donor template, are introduced at the site of the break. These nucleotide changes can range from single base pair changes to insertions of entire genes or even large cassettes of multiple genes (23, 24).

We have designed and validated HDR-mediated genome editing systems and shown that they can modify human CD34+ HSPCs ex vivo by insertion at the HBB locus of an anti-sickling β-globin cDNA. We will adapt these systems for in vivo delivery, and test their efficacy and efficiency for targeted gene-editing of HBB using a previously described mouse model in which the mouse globin genes are replaced by human alleles encoding HbA and HbS (18). These SCD mice recapitulate many features of human SCD, including red blood cell sickling and aggregation in the vasculature, splenic and vascular abnormalities, anemia, and defects in kidney function. Thus, they provide an appropriate pre-clinical platform in which to test the efficacy of gene editing complexes that target the human HBB gene in a physiologically relevant animal model of SCD. Our approach represents a significant innovation and improvement over prior attempts at HBB gene correction in two respects: (1) we will develop a single, generic method to functionally correct a wide variety of mutations in the β-globin coding sequence, and (2) we will accomplish HDR-mediated gene modification in endogenous HSPCs, without a requirement for cell isolation or transplant.

Our focus on gene correction in endogenous HSPCs, in particular, has the potential to overcome two key limitations faced by similar approaches that rely on HSPC isolation, ex vivo modification, and subsequent transplantation. First, strong data indicate that the only cells capable of long-term hematopoietic reconstitution following transplant are the most primitive subset of hematopoietic stem cells (LT-HSCs); however, multiple recent studies indicate that the engraftment efficiency of these cells is reduced following ex vivo manipulation, leading to reduced representation of gene-modified cells in the reconstituted hematopoietic systems of transplant recipients (9, 12, 17). By accomplishing HSC editing in situ, our strategy avoids the need for transplantation, thereby circumventing this “engraftment problem”. Second, recent data tracing the in vivo clonal dynamics during steady-state hematopoiesis has raised the possibility that the unique function of HSCs as hematopoietic regenerative units may be restricted to the transplant setting, and that endogenous hematopoiesis may be supported largely by a collection of very long-lived, lineage-restricted progenitor cells (25, 26). Given the higher rates of cell division observed for these progenitors, it is possible that they may be more amenable to HDR-based gene editing; however, since they fail to engraft long-term following transplantation (16), they have not been considered as viable targets for ex vivo gene editing approaches. Our strategy of in vivo editing could overcome this limitation by enabling the modification of such long-acting progenitor cells in situ, where their long-term activity is preserved, thereby providing an alternative or additional source of modified regenerative cells for therapy. Our approach is also robustly supported by the following key Preliminary Data:

Targeting HBB using β-globin-specific nucleases.

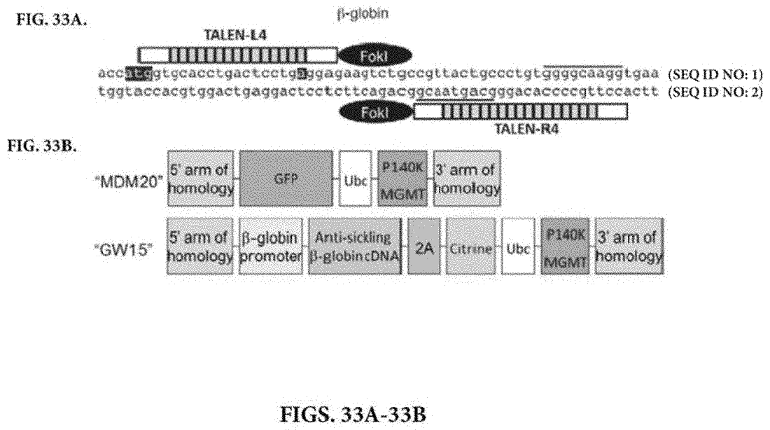

To test clinically-relevant β-globin-specific nucleases that effectively target the HBB locus in human hematopoietic precursor cells, we introduce HBB-directed nucleases, with or without a β-globin template DNA constructed to introduce a fluorescent reporter under the control of the endogenous β-globin promoter, into human CD34+ HSPCs (FIG. 33). Our initial studies employed the Transcription Activator-Like Effector Nuclease (TALEN) system, originally adapted from the plant bacterial pathogen Xanthomonas (27). TALENs are engineered, programmable nucleases composed of a specifically designed DNA binding domain fused to the FokI endonuclease domain (28). Binding of a pair of TALENs to contiguous sites in DNA allows for dimerization of the associated FokI domains and generation of a double strand break (DSB) near the TALEN binding site. This break can be repaired by mutagenic NHEJ, or, if a homologous DNA template is available, by HDR. A recently published study (11) identified four candidate left (L1-L4) and right (R1-R4) TALEN binding sites near the sickle mutation site in HBB, and generated eight individual TALENs directed at these sites. Combinatorial testing of these TALEN pairs revealed the L4-R4 pair (FIG. 33A) to have superior activity (11). This TALEN pair further stimulated high rates of HDR at the HBB locus in transfected K562 cells (a human erythroleukemia cell line), yielding stable integration of a donor plasmid with 5′ and 3′ HBB homology regions in up to 20% of transfected cells (11).

TALEN-Directed Homologous Recombination (HR) at HBB in Human HSPCs.

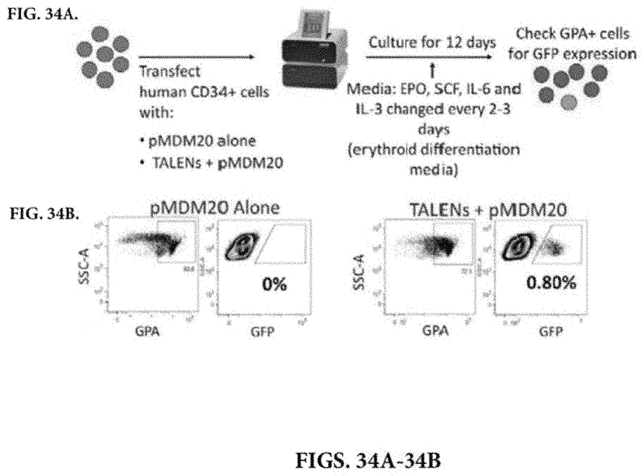

To assess the feasibility of HBB targeting and HDR in primary human CD34+ HSPCs, we nucleofected human CD34+ bone marrow (BM) HSPCs (FIG. 34A) with plasmid DNA encoding TALENs L4 and R4, together with a β-globin template DNA (MDM20, FIG. 33B) that introduces GFP under control of the endogenous β-globin promoter. In this system, cells will express GFP only after HDR with the donor template and only upon induction of adult hemoglobin expression. HSPCs were cultured after nucleofection in erythroid differentiation media (StemSpan media containing EPO, SLF, IL-3, and IL-6), and after 7 days, a subset of the cultured cells exhibited high levels of the erythroid markers CD71 and CD235a (also known as Glycophorin A (GlyA)) and began to express hemoglobin (data not shown). Excitingly, a low, but detectable fraction (˜0.1%) of cells in cultures initiated after transfection with the HBB-targeting donor template and β-globin TALENs became GFP+; whereas untransfected control cells and cells receiving donor template alone showed no GFP expression (data not shown). GFP expression was further increased in CD235a+ erythroid cells after 12 days of culture in cells receiving both TALENs and template, and remained undetectable in CD235a+ cells from control cultures (FIG. 34B). These results confirm that β-globin specific nucleases can stimulate HDR with a β-globin directed template in human HSPCs, generating stably transfected progenitor cells that can differentiate to produce β-globin expressing erythroid lineage cells. Considering data from multiple different donors (>8), we estimate the efficiency of gene modification in this ex vivo system at −0.2-1.0% of input cells. Supporting the robustness of this approach, another lab has obtained the same frequency of gene targeting in CD34+ cells at two different gene loci associated with severe combined immunodeficiency ((29) and data not shown).

Enrichment of HBB Targeted Human HSPCs by Drug Selection.

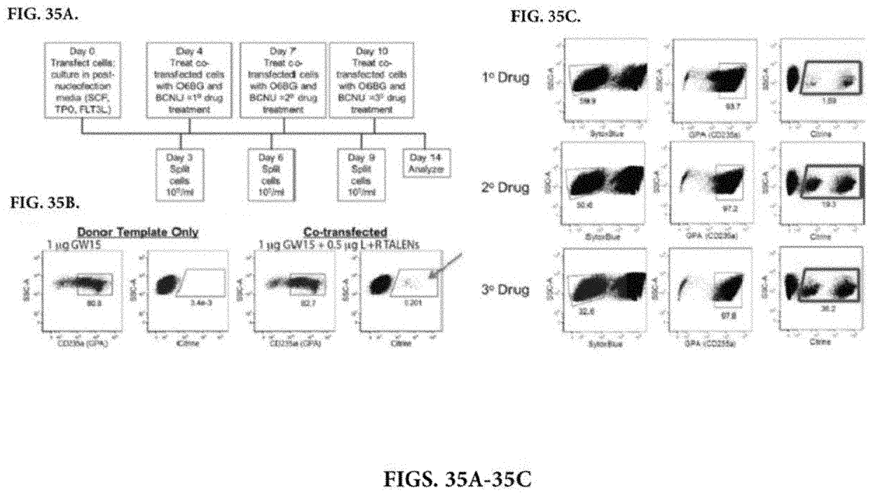

Our original donor templates (both MDM20 and GW15) were engineered to include a drug selection cassette encoded by the P140K variant O(6)-methylguanine-DNA methyltransferase (MGMT) and constitutively expressed from the human ubiquitin C (Ubc) promoter (see FIG. 33B). Several publications indicate that this drug resistance cassette can be used to enrich for cells that have undergone HDR with the donor template, both in vivo and in vitro (30-33). This selection strategy also has a safety profile compatible with use in a phase I clinical trial involving glioblastoma patients (34). Expression of P140K MGMT enables drug selection using 06-benzylguanine (BG) in combination with 1,3-bis(2-chloroethyl)-1-nitrosourea (BCNU) (30-33). Preliminary studies using the MDM20 vector indicated that a portion of cells transfected with β-globin donor template and TALENs were resistant to cell death when exposed to BG/BCNU treatment (data not shown). Furthermore, when human HSPCs were co-transfected with the anti-sickling β-globin template GW15 (FIG. 33B) and L4-R4 TALENs and subjected to three rounds of BG/BCNU selection, the resulting cultures of erythroid lineage cells showed increased representation of HBB-targeted cells at each round of selection, with one experiment yielding a remarkable 180-fold enrichment of HBB-targeted cells (detected by flow cytometry for the introduced β-globin responsive fluorescent marker citrine, FIG. 35). Importantly, citrine was expressed only in GPA+ erythroid lineage cells, reflecting control by the β-globin promoter. The enrichment of citrine+ cells by drug selection confirms the stable integration of the donor template in HSPCs. Furthermore, the fact that no GPA-negative (non-erythroid) cells were citrine+(data not shown), even after long-term culture, confirms the fidelity of our fluorescent reporter system.

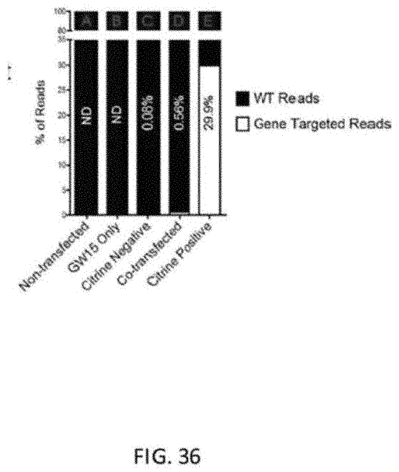

Confirmation of HBB targeting in human HSPCs by sequencing analysis. Finally, to validate specific targeting and modification of HBB in human HSPCs at the genomic and transcriptomic levels, we performed RNA and DNA sequencing analysis using single molecule real time (SMRT) sequencing, which provides an affordable, rapid, and high-throughput method for analysis of the β-globin locus following TALEN treatment (35). As a first experiment, we isolated 5 populations of cells for comparison by DNA sequencing (FIG. 36): A, untransfected HSPCs (NON-TRANSFECTED); B, HSPCs transfected with donor template only (GW15 ONLY); C, HSPCs transfected with GW15+L4-R4 TALENs and sorted for lack of citrine expression (CITRINE NEGATIVE); D, HSPCs transfected with GW15+L4-R4 TALENs, but not sorted or selected with BG/BCNU (CO-TRANSFECTED); and E, HSPCs transfected with GW15+L4-R4 TALENs, subjected to 2 rounds of BG/BCNU selection, and then sorted for Citrine-positive cells (CITRINE POSITIVE). (Please note, we could not sort citrine+ cells for sequencing analysis prior to drug selection because the low frequency of these cells (0.5% in this experiment) prevented us from obtaining sufficient cell numbers.) This sequencing analysis confirmed appropriate genomic integration of the donor construct at the HBB locus, and showed a direct correlation between the percent gene-targeted reads and the percent citrine positivity in samples analyzed by flow cytometry (FIG. 35 and data not shown), with sorted citrine+ cells after secondary drug selection exhibiting almost 30% gene targeted reads.

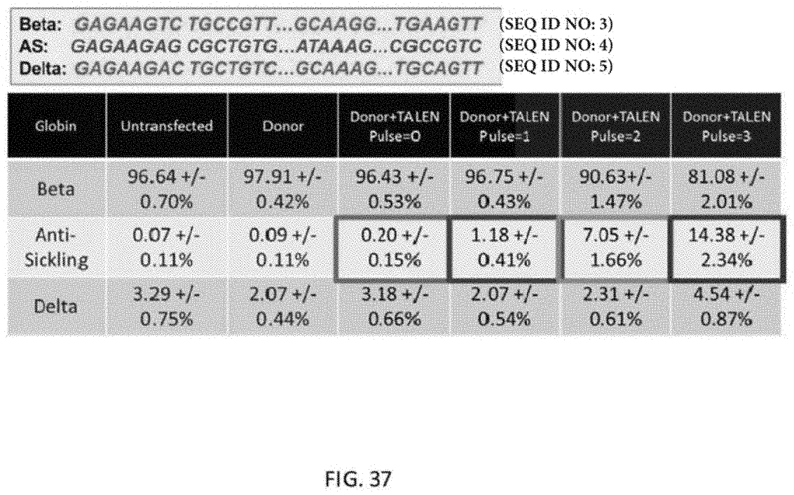

Next, in a separate series of experiments, we analyzed un-sorted pools of untransfected HSPCs, or HSPCs transfected with donor template only or with donor+TALENs for expression of various forms of globin mRNA. In particular, we defined four unique sequence ‘signatures’ that allowed for mapping of RNA sequencing reads to either endogenous β-globin, endogenous δ-globin (which shares significant sequence homology with β-globin) or the HR-introduced variant anti-sickling β-globin (which contains both the Thr→Val substitution (21) and multiple wobble mutations, FIG. 37). As shown in FIG. 37, anti-sickling β-globin is essentially undetectable in untransfected and donor only cultures, but represents a low percentage of total globin reads in unselected cultures (Pulse=0) of HSPCs co-transfected with donor+TALENs. Importantly, the fraction of globin reads accounted for by the donor vector delivered anti-sickling β-globin increased sequentially with each round of drug selection, paralleling increases in the % citrine-positive cells (FIG. 35 and data not shown) and further confirming stable integration of the targeting construct.

Gene Editing in SCD Patient-Derived HSPCs.

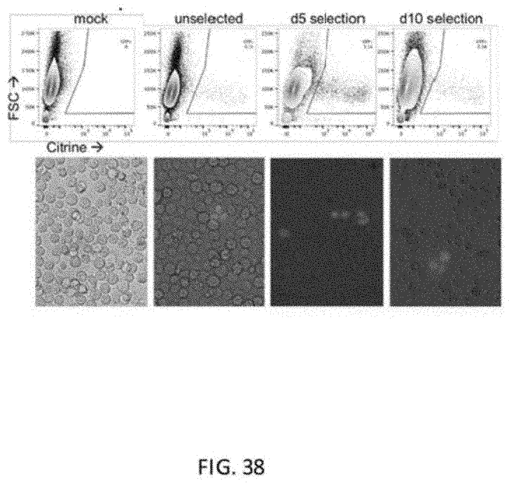

To verify that genome modification of human HSPCs is likewise effective in HSPCs from β-hemoglobinopathy patients, we isolated CD34+ HSPCs from the banked umbilical cord of a single SCD patient. SCD HSPCs were nucleofected with L4-R4 TALENs+ the GW15 donor plasmid, and then cultured in media containing erythropoietic cytokines, with or without MGMT selection. Citrine+ cells expressing the erythroid markers GPA and CD71 were apparent in both the unselected cultures and in cultures in which transduced cells were subjected to drug selection, but were absent from mock transfected cultures (FIG. 38). Molecular analyses of these cells (as in FIGS. 36 and 37) is ongoing. These data are consistent with published work from Kohn and colleagues using HBB ZFNs (9), and from the Porteus lab using CRISPR/Cas9 (1), and confirm that site directed genome editing at HBB can be accomplished in SCD cells and results in proper integration of our therapeutic cDNA construct.

Preliminary data (FIGS. 35-38), demonstrates the feasibility of HDR-mediated gene editing in human CD34+ HSPCs, including HSPCs from SCD patients ((1, 9) and FIG. 38); however, achieving robust engraftment of gene-edited cells after ex vivo manipulation remains a significant challenge for translating these exciting advances. Moreover, even if efficient engraftment were achieved following ex vivo editing, the transplant procedure itself carries significant risk to the patient, including conditioning-related toxicities and risk of graft failure. For these reasons, development of an HBB editing approach that does not rely on transplantation is highly desirable. In the studies proposed here, we will test the feasibility of such a transplant-independent approach using an AAV-based delivery system to introduce HBB editing components into HSPCs in situ. AAVs are attractive delivery vectors due to their prevalence and general non-pathogenicity in human populations (36, 37) and their prior approval for use in clinical trials (38, 39). AAVs also provide the opportunity for both local and systemic delivery of virally encoded gene editing complexes. However, the limited packaging capacity of AAVs (4.7 kb) presents an obstacle for their use in delivering large sequences of DNA, such as the L4-R4 TALENs, which have a combined size of >6 kB. To overcome this problem, we have transitioned to use of Clustered Regularly Interspaced Short Palindromic Repeats (CRISPR/Cas9, also known as RNA-guided endonucleases or “RGENs”) (27, 40, 41) for HBB gene editing.

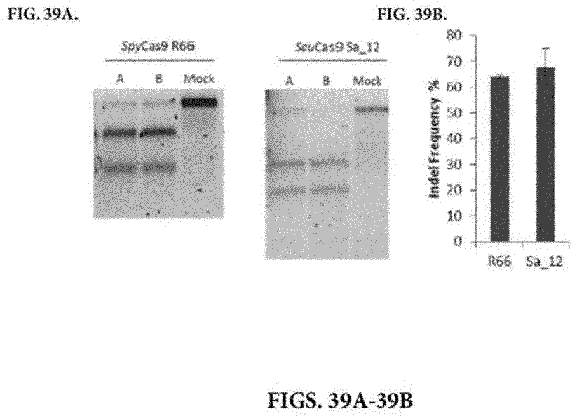

The CRISPR-Cas9 system is a recent development in genome engineering first adapted from Streptococcus pyogenes (Sp), and subsequently from other bacteria including Staphylococcus aureus (Sa, (42)). CRISPR-Cas9 RGEN systems consist of the Cas9 endonuclease and a programmable guide RNA (gRNA). Cas9-gRNA probes the genome for protospacer-adjacent motifs (PAM) (-NGG for SpCas9 (43) and -NNGGR(T) for SaCas9 (42)). Upon gRNA:DNA base-pairing, Cas9 creates a double-strand break (DSB) in the DNA that induces genetic change. CRISPR/Cas9 RGENs have been used to target both expressed and non-expressed genes in multiple cell types from multiple organisms both in vitro (44-48) and in vivo (49, 50). Recent data further demonstrates the utility of SaCas9 for multisystemic gene targeting of many different cell types in vivo, including hepatocytes, muscle fibers, cardiomyocytes, and muscle regenerative stem cells (2, 42, 51, 52) (see below). Finally, studies indicate that HBB-directed RGENs actually produce higher frequencies of gene disruption than TALENs and stimulate higher frequencies of HDR when introduced together with an appropriate donor template in the erythropoietic cell line K562 or human HSPCs ((1, 29) and unpublished data). Indeed, recently published work successfully applied CRISPR/Cas9 for ex vivo targeting and correction of the β-globin gene in human HSPCs (1). gRNAs (R66 and Sa_12) appropriate for use with Sp or SaCas9, respectively, have been designed and validated for targeting near the sickle mutation in HBB exon 1 in hematopoietic cell lines (FIG. 39) and HSPCs (data not shown). In silico analysis using the Cas-Offinder tool suggests minimal predicted off-target activity for either gRNA in the human genome (not shown).

AAV Transduction and CRISPR Reporter System in Mice

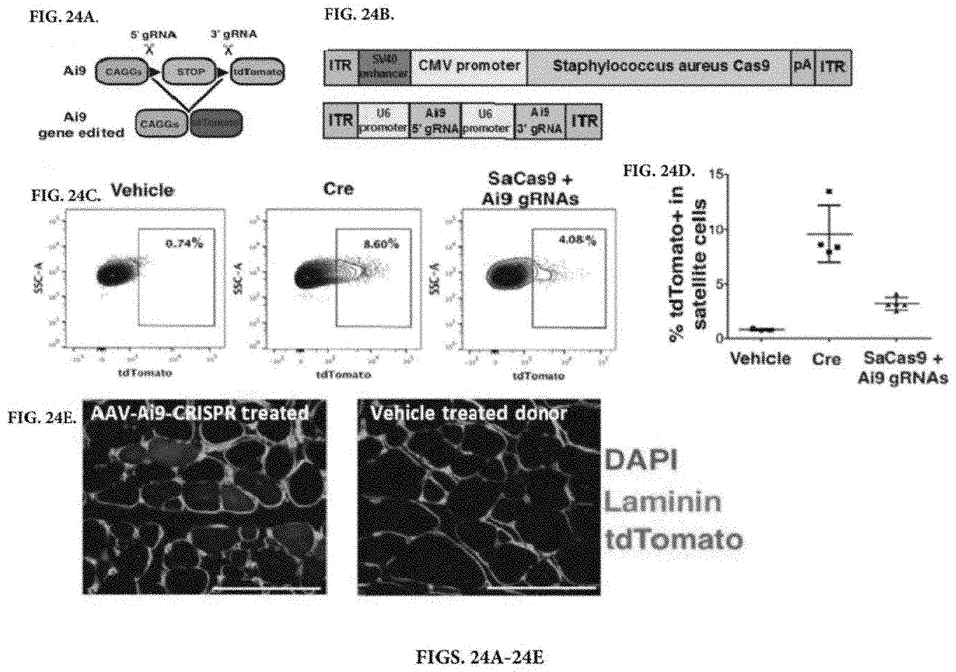

We also have designed and validated a novel fluorescence-based AAV transduction and CRISPR reporter system in mice using the Ai9 loxP mouse strain (JAX strain #007905) (2). The Ai9 mouse harbors a genomically integrated loxP-3xSTOPSV40 pA-loxP (LSL) cassette flanked by an upstream constitutive CAG promoter and downstream tdTomato gene in the Rosa26 locus (53) (FIG. 24A). TdTomato is not normally expressed in this mouse due to the upstream terminators present in the transgene (3×STOP); however, in the presence of Cre recombinase, or of active CRISPR complexes containing gRNAs targeting near the 5′ and 3′ loxP sites (2), the LSL cassette is excised and tdTomato expression is activated (FIG. 24A). In a recent publication (2), we used this system to demonstrate effective AAV transduction and delivery of Cre or CRISPR complexes into endogenous stem cells in skeletal muscle, as well as multinucleated muscle fibers and cardiomyocytes. For that study, we produced AAVs (using the muscle-tropic serotype 9 (54)) encoding SaCas9 and Sa gRNAs targeting the Ai9 locus (Ai9 gRNAs) or exon 23 of the endogenous Dmd gene (Dmd23 gRNAs). Our strategy employed a dual AAV system (FIG. 24B), which yielded superior editing efficiencies as compared to a single vector system (due to AAV packaging limitations) (2). We then injected AAV-Cre or AAV-CRISPR intramuscularly or systemically into recipient Pax7-zsGreen+/−;mdx;Ai9 mice, which carry a nonsense mutation (mdx) in Dmd exon 23 as well as the Ai9 reporter allele and a fluorescent marker (Pax7-zsGreen) that identifies muscle stem cells. FACS analysis 2 wks later revealed tdTomato expression in Pax7-ZsGreen+ muscle stem cells following local or systemic delivery of AAV-Cre or AAV-Ai9 CRISPR (FIG. 24 C,D and (2)). In vitro differentiation of FACS-isolated ZsGreen+ muscle stem cells from mice receiving intramuscular or systemic AAV-Cre or AAV-Ai9 CRISPR produced tdTomato+ myotubes, and transplantation of freshly isolated stem cells into recipient mdx muscles showed engraftment of tdTomato+ muscle fibers in vivo (FIG. 24E and (2)). Similar editing in endogenous muscle stem cells by local or systemic AAV-CRISPR paired with gRNAs targeting the endogenous Dmd gene was also documented, and led to recovery of Dystrophin protein expression in satellite cells and their progeny (2).

Together, these data confirm the utility of the Ai9 system to provide sensitive, fluorescence-based detection of CRISPR activity in mice with single cell resolution, the capacity to accomplish in vivo genome modification of endogenous stem cell populations, and the feasibility of prospective detection and isolation of gene-edited stem cells and their progeny by fluorescence activated cell sorting (FACS) (FIG. 24C,D). They further document the preservation of engraftment and differentiation potential in AAV-transduced and in vivo gene-edited stem cells (FIG. 24E and (2)). These results strongly suggest that other disease-relevant stem cell populations, including HSPCs, will likewise be editable in vivo, provided the correct combination of targeting vectors and delivery strategies can be identified.

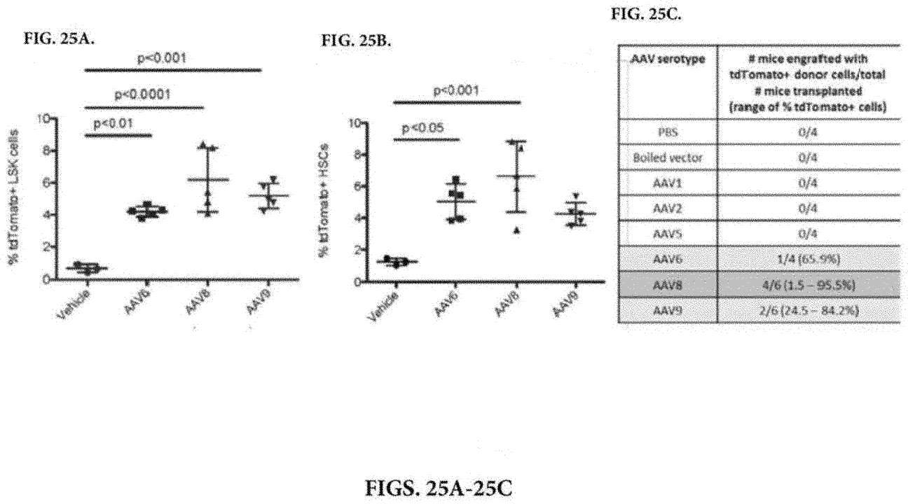

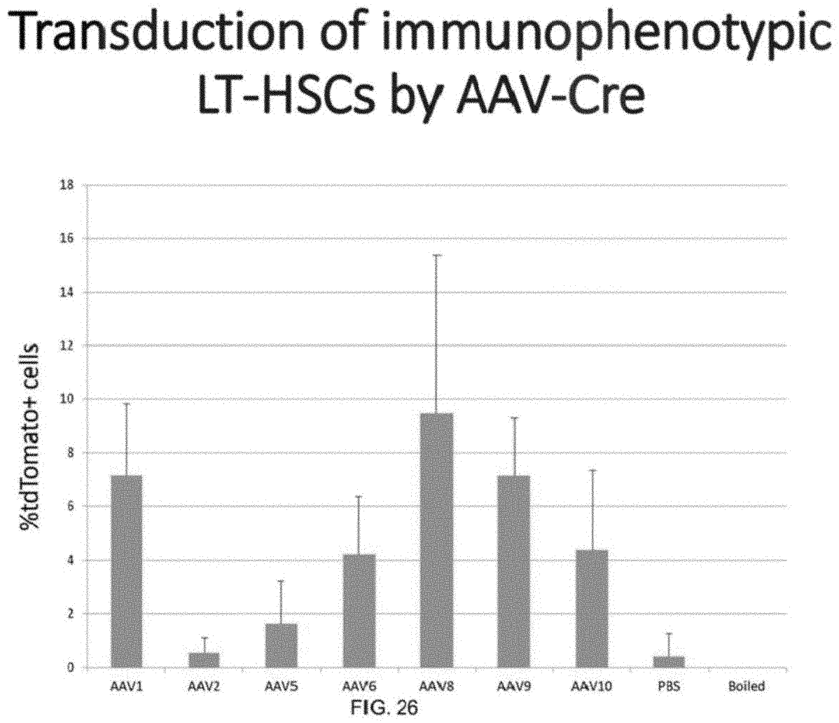

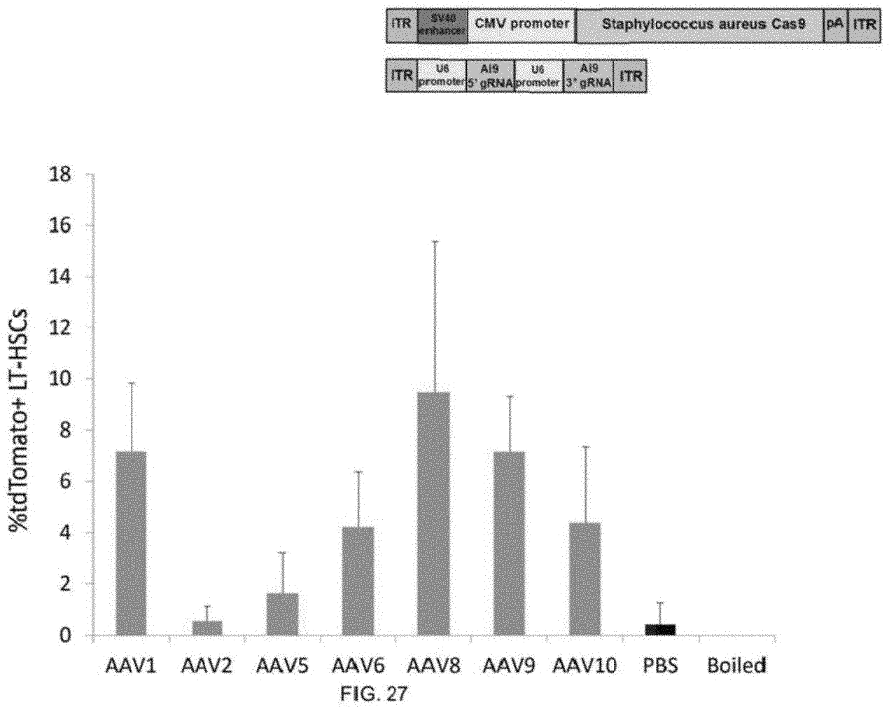

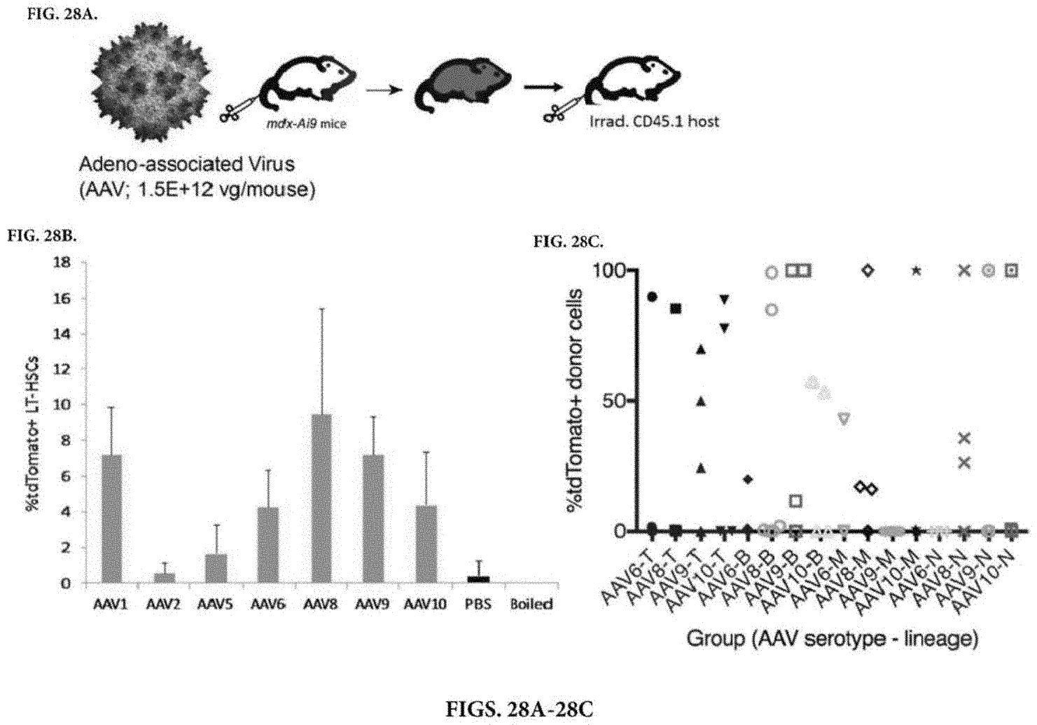

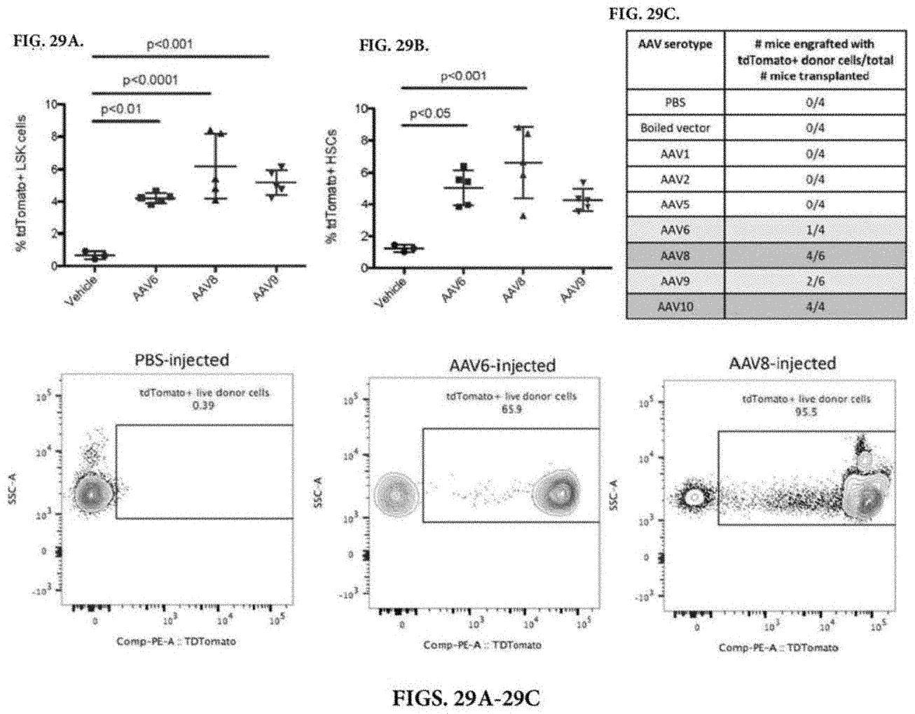

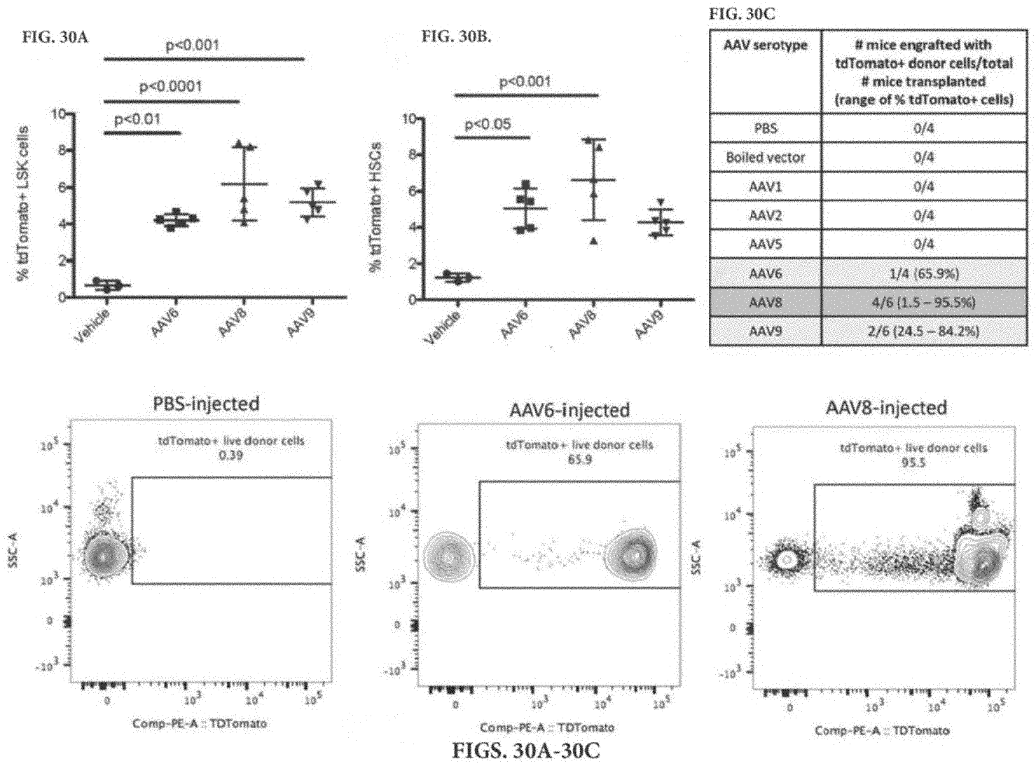

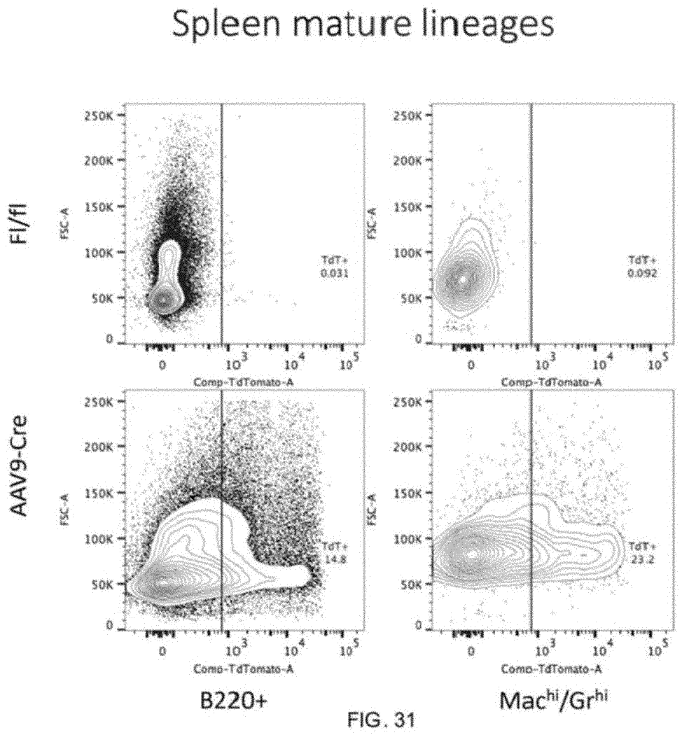

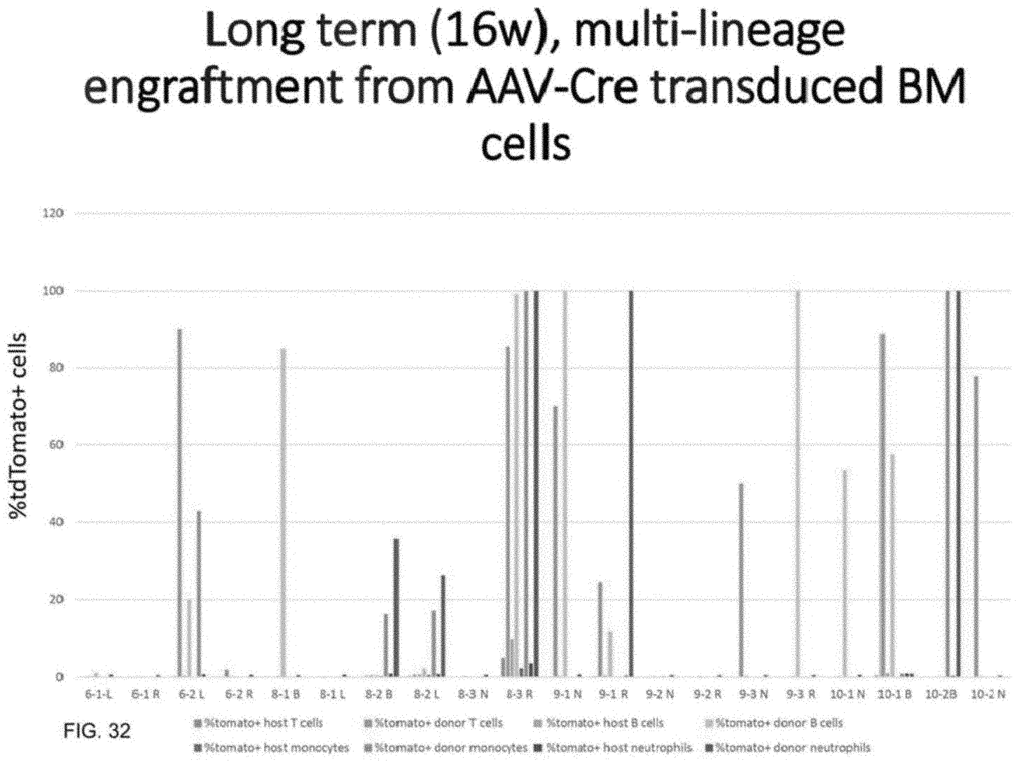

Supporting this notion, we performed preliminary studies (FIG. 25) using the AAV-Cre Ai9 system described above to identify AAV serotypes and delivery routes supportive of in vivo HSPC transduction and modification. This work demonstrates that HSPCs, including long-term reconstituting HSCs, in normal mice can be effectively transduced by multiple AAV serotypes following either intravenous or intrafemoral injection, with the highest rates of transduction achieved with AAV8 (FIG. 25A, 25B). Importantly, transplantation of tdTomato+ HSPCs from these mice confirm that the transduced and modified cells retain long-term, multilineage reconstituting capacity (FIG. 25C), documenting the utility of this system to achieve successful targeting of HSPCs in vivo that maintain the capacity to replenish the blood system with gene-edited cells.

Evaluating in vivo HSPC gene editing frequencies using AAV-Ai9 CRISPR. Our preliminary studies using AAV-Cre clearly establish the feasibility of in vivo transduction and permanent genomic modification of mouse HSPCs, including long-term reconstituting HSCs, by multiple AAV serotypes in a substantial fraction (up to 9%) of HSCs in otherwise unperturbed mice (FIG. 25). Furthermore, they have identified AAV8 as the most efficient serotype for HSPC transduction and demonstrated that systemic delivery is as efficient as intrafemoral injection (data not shown). These data provide strong justification for proceeding to use this sensitive reporter system to test HSPC gene editing by AAV-CRISPR, as we have done previously for endogenous muscle stem cells (2). These experiments will utilize a dual vector system (FIG. 24B and (2)) with SaCas9 expressed from one AAV and Ai9 gRNAs encoded in the other. Our prior work documents that this dual system exhibits a similar efficiency of in vivo gene editing as single vector designs (2), and regardless, dual vectors may be required for adaptation of the CRISPR system to HDR since AAV packaging limitations (4.7 kb) prohibit incorporation of the HDR homology template together with SaCas9 and gRNAs within a single AAV vector.

As in our recent publication (2), AAV vectors is produced and provided by the Massachusetts Eye and Ear Infirmary (MEEI) Gene Transfer Vector Core (http://vector.meei.harvard.edu/). Ai9 mice (n=8 per experimental condition, currently breeding in our animal colony) will be injected intravenously with AAV8-SaCas9 and AAV8-Ai9 gRNAs vectors over a 2 log range of titers (10¹¹, 10¹² or 10¹³ vg/mouse, chosen based on recent and prior publications (54-57), including our own (2), indicating their relevance for in vivo studies). To account for possible effects of age and HSPC activation state (e.g., quiescent or proliferating) on AAV transduction and genome modification, we will compare editing rates in otherwise unperturbed early postnatal (P3) and adult (P42) Ai9 mice, and in adult Ai9 mice treated with pharmacologic mobilizing agents (cyclophosphamide(Cy)/G-CSF). We chose the Cy/G-CSF protocol for these studies because we have extensive experience with this treatment regimen (58-60) and know that it induces proliferation of all endogenous HSCs in mice, with well-defined topology and kinetics (58). Importantly, while we recognize that induction of mobilization may not be desirable in patients, and indeed, based on our preliminary data (FIG. 25) is unlikely to be necessary, we also believe that evaluating this experimental perturbation in these pre-clinical studies will provide important information about the biological influences on AAV transduction and gene editing rates in vivo that will be useful for other applications of this system as an experimental platform for in situ modification of HSPCs in hematopoiesis research. AAVs will be administered to Cy/G-treated adult mice at time points preceding (day 0 and day +1), concurrent with (day +2) and following (day +3) this peak of mobilization-induced HSC proliferation. Control mice (n=8) will receive AAV-SaCas9+ irrelevant (e.g., Dmd (61)) gRNAs. In all cases, AAV-Ai9-CRISPR editing efficiency will be read out at 4 wks. after AAV administration by flow cytometry (for tdTomato+ cells) performed on HSPCs (see FIG. 25). We will use standard immuno-phenotypic markers, including primitive LT-HSCs (Lin-ckit+Sca1+(LKS) CD150+CD48−), multipotent progenitors (MPPs; LKS CD150-CD48+), common myeloid progenitors (CMPs; Lin-ckit+Sca1-CD34highCD16/32−), and megakaryocyte-erythrocyte progenitors (MEPs, Lin-ckit+Sca1-CD34lowCD16/32−) (16) to delineate multipotent and oligopotent hematopoietic precursor cells, and compare frequencies of TdTomato+ cells in each of these subsets across ages and mobilization conditions. Analysis of transduction and editing rates in non-hematopoietic organs (e.g., liver, muscle, heart, etc.) will also be compared, using fluorescence microcopy to quantify tdTomato+ cells. The 4 wk. time point was chosen based on preliminary studies indicating that this is sufficient time to allow for genomic excision of the transgenic STOP cassette and tdTomato expression (FIGS. 24 and 25 and (2)). We also have confirmed that HSPC surface marker phenotypes are unaltered at this time point, consistent with prior observations that AAV vectors do not induce sustained or significant inflammation (62). The permanence of gene editing of LT-HSCs observed by FACS will be confirmed by hematopoietic reconstitution studies, in which tdTomato+ CD150+CD48-Lin−Sca1+ckit+ HSCs will be isolated and transplanted into irradiated, congenic (differing in allotypic expression of the pan-hematopoietic cell surface maker CD45.1) recipient mice, as in (59, 63-65) and FIG. 25. Primary recipients (n=5 recipients per donor) will be analyzed for multi-lineage reconstitution by tdTomato+ cells 4, 8 and 20 wk. post-transplant, and then marrow cells from these mice will be used in secondary transplants to confirm engraftment by LT-HSCs.

These studies will require 16 Ai9 mice (experimental+control) and up to 80 congenic CD45.1 recipients) for each of 3 replicate experiments per condition (a total of 288 mice), and will test 6 experimental conditions (2 ages and 4 Cy/G time points) at 3 viral titers. Thus, this work will require 288×6×3=5184 experimental animals. For all studies, male and female mice will be used equally and randomized to experimental group. Analyses will be performed by an observer blinded to sample identity.

Adaptation of AAV-CRISPR to HDR for HBB.

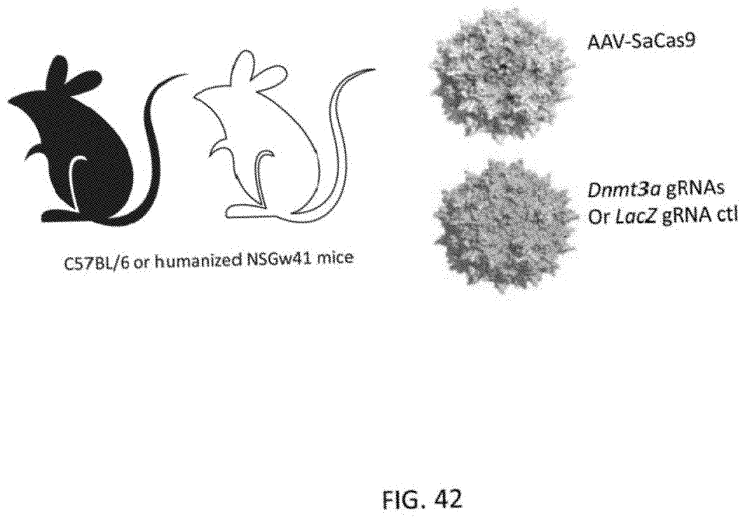

We will test the feasibility in a pre-clinical model of using in vivo delivery of gene editing complexes to endogenous HSPCs to recover expression of normal HbB in cells harboring mutations that lead to β-hemoglobinopathy. The studies described above will provide a critical (and yet independently useful) step towards this goal by establishing optimal strategies for AAV-mediated delivery of active gene editing complexes to HSPCs in vivo. As a next step, we will test the utility of this approach to accomplish HDR, as this would allow for functional correction of the mutated HBB gene using a common targeting strategy that could be applied broadly across the wide spectrum of HBB mutations underlying SCD and β-thalassemia. To do this, we will use the “Townes model” (ha/ha::βS/βS (18)) SCD mice. These mice carry multiple human hemoglobin knock-in alleles, which replace the endogenous mouse α-globin genes with human hemoglobin a (ha) and replace the endogenous mouse major and minor β-globin with human hemoglobin gamma (Aγ) and sickle hemoglobin beta (βS) (this allele is also known as −1400 γ-βS). ha/ha::βS/βS mice (hereafter “SCD mice”) are viable and fertile, but exhibit red blood cell sickling and aggregation in blood vessels, splenic and vascular abnormalities, anemia, and defects in kidney function—all phenotypes that mimic human SCD. Importantly, animals that carry only 1 βS allele (ha/ha::βA/βS mice) are protected from these phenotypes, similar to human βS heterozygotes. Thus, the presence of human sickle alleles that can be targeted by our existing human HBB gRNA (Sa_12, FIG. 42), together with the phenotypic similarity to human SCD, make the Townes SCD mice an excellent pre-clinical model in which to test the therapeutic potential of our strategy for in vivo genome editing in HSPCs.

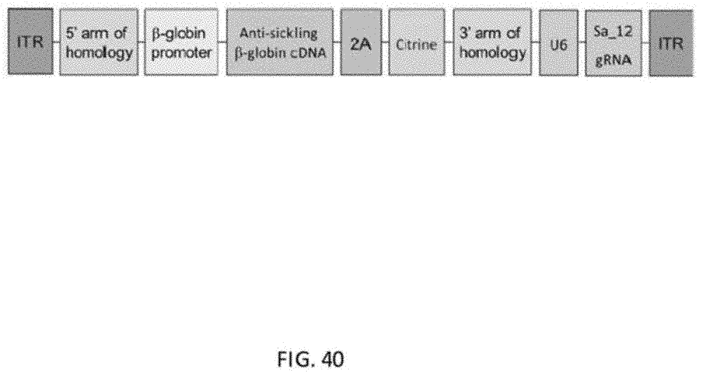

SCD mice (18) will be injected with AAV particles carrying SaCas9, the HBB-targeting gRNA Sa_12, and a modified donor construct (hereafter “AAV-HBB-CRISPR”). Again, we will employ a dual AAV system, for incorporation of all components. The first AAV will carry SaCa9, driven by a strong CMV promoter (see FIG. 24B and (2)). The second AAV will carry both the donor template and Sa_12 HBB gRNA, driven by the U6 promoter (FIG. 40). Importantly, the feasibility and utility of this strategy for in vivo HDR is strongly supported by our prior work with a similar system (FIG. 24 and (2)), which showed superior DNA cutting with dual versus single vector systems, and by a recent paper that used the same dual AAV approach for HDR to functionally correct the OTC gene in hepatocytes of mice with ornithine transcarbamylase deficiency (56).

The HBB donor template we will use for these studies is very similar to that described above and allows integration with a partial β-globin promoter of a variant anti-sickling human β-globin cDNA (8-11) containing both the Thr→Val substitution (21) and multiple wobble mutations (synonymous substitutions) that disrupt the Sa_12 PAM and seed region to prevent re-cutting and allow discrimination from the endogenous βS allele (FIG. 38). Donor sequence insertion into the HBB locus is required for β-globin cDNA expression and also allows β-globin promoter dependent expression of fluorescent citrine, encoded 3′ of the anti-sickling β-globin and separated by a self-cleaving 2A peptide sequence (FIG. 40 and see FIGS. 33-38).

AAV8-HBB-CRISPR will be administered to early postnatal (P3) or adult (P42) SCD mice according to the optimal methods established in our studies with AAV-Cre (FIG. 24) and AAV-Ai9-CRISPR (see above; n=10 SCD mice per experimental condition). Equal numbers of age- and sex-matched animals injected via an identical approach with AAV-SaCas9 only, AAV-GW25 only (encoding the donor template and Sa_12 gRNA, FIG. 40), or dual AAV-SaCas9+AAV-Sa_12 gRNA (lacking donor template) will be used as controls. Animals (n=240 mice; 10 mice per group×4 experimental groups×2 ages×3 replicate experiments) will be randomized prior to study entry. Both males and females will be used. Four weeks after AAV injection, expression of donor-encoded citrine will be assayed in mature peripheral blood myeloid, lymphoid and erythroid lineages, using flow cytometric analysis and co-staining for relevant lineage markers (e.g. M1/70, Ly6G, CD3, CD19, Ter119, and CD71) (see FIG. 23). 12 wks. after transplant, we will harvest bone marrow from these mice for immunophenotypic analysis of splenic and bone marrow HSPCs as well as mature peripheral blood lineages, using the markers indicated above (16). Citrine+ and citrine-subsets of each of these mature lineages (myeloid, lymphoid and erythroid) and progenitor subsets (LT-HSCs, MPPs, CMPs, MEPs) will be sorted by FACS and subjected to genomic and transcriptomic analysis to determine the frequency of on-target HDR, using SMRT sequencing and droplet digital PCR (see FIGS. 36 and 37 above and (29)) and the frequency of mutagenic events (i.e., insertions and deletions (indels) generated by NHEJ mediated repair of CRISPR-Cas9 induced DSBs at HBB or at other predicted off-target genomic loci). Prediction of potential off-target sites in the mouse genome for the Sa_12 HBB gRNA will be performed using Cas-Offinder (http://www.rgenome.net/cas-offinder/). Of note, prior analysis in the human genome identified only 12 sites with a 4 bp mismatch and 0 sites with mismatches <4 (unpublished) for this gRNA, consistent with the longer SaCas9 PAM (42), which results in fewer closely matched sites genome-wide.

Importantly, because DSBs introduced by activity of our CRISPR-Cas9 gene editing tool at the HBB locus can be repaired by either HDR or NHEJ, editing at this locus can result in multiple different outcomes, depending on the number of alleles affected and the type of modification(s) introduced. Gene editing in SCD cells, which harbor two βS alleles, could result in 6 different outcomes (Table 1). Thus, in addition to assessing off-target (non-HBB) modifications in CRISPR-Cas9 modified cells, it will be important to determine the relative frequencies with which each of these possible on-target genomic modifications may occur. Notably, because patients with SCD produce a pathogenic beta hemoglobin chain, whereas patients with β-thalessemia experience ineffective erythropoiesis due to insufficient HbB production, conversion of a subset of SCD HSPCs to a genotype consistent with β-thalassemia, sickle β-thalassemia, or sickle trait is unlikely to cause significant complications. Genomic assessments of the relative frequency of on-target HDR and on- and off-target indels will be performed using genomic DNA harvested from FACS sorted mature cells and HSPCs and analyzed by SMRT sequencing, Illumina deep sequencing and droplet digital PCR, as in (2, 17, 29). Briefly, for determining on-target gene editing rates, nested PCR will be used to prepare samples for sequencing on the Illumina MiSeq platform.

TABLE 1

| | | Hb | Resultant | |
|---|---|---|---|---|
| | | proteins | phenotype | Impact of |
| β^{S} allele 1 | β^{S} allele 2 | produced | conversion | modification |
| No | No | AS | SCD →SCD | No change in |
| modification | modification | | (no phenotypic change) | pathology |
| Indel | No | AS | SCD →Sickle beta | No change in |
| (inactivation) | modification | | thalassemia (essentially | pathology |
| | | | the same as SCD; no | |
| | | | phenotypic change) | |
| Indel | Indel | A | SCD →β-thalassemia | No HbB |
| (inactivation) | (inactivation) | | major (microcytic anemia) | produced |
| HDR | No | AB | SCD →Sickle trait | Therapeutic |
| | modification | and AS | (benign condition) | |
| HDR | Indel | AB | SCD →β thalassemia | Therapeutic |
| | (inactivation) | | minor | |
| HDR | HDR | AB | SCD →Normal | Therapeutic |

Table 1: Possible outcomes for individual HSPCs of gene editing at HBB and resultant phenotypic conversions. Each of these possible modifications may be represented in the pool of edited cells at different frequencies. As cells expressing normal Hb have a tremendous selective advantage in SCD models (4, 66), the presence of some unmodified clones (Rows 1-2) or clones that carry heterozygous or homozygous disruption of HBB should not cause additional pathology in SCD mice (or patients). The presence of cells with recovered expression of normal HbB (rows 5-7) would be therapeutic.

Results will be analyzed based on the PERL programming language, and specifically designed to quantify indels and HDR events at predetermined genomic locations (67-69). This approach is extremely sensitive and specific, but could be challenging if the AAV-donor template vector remains in any of the sorted cell populations, as this would cause high background due to contaminating amplification. Thus, to complement the Illumina approach, we will also use a PacBio SMRT sequencing platform. Although SMRT sequencing has lower throughput and a higher error rate, it allows for sequencing of longer amplicons, which permits specific amplification of genomic DNA with PCR primers that bind outside the donor homology arms. The Bao lab has successfully developed an analysis pipeline for quantifying genome editing events from SMRT sequencing data that addresses the error rate issues (29). Results from these experiments will allow us to quantify the level of each type of modification within each sorted bulk population. To further determine allelic frequencies of HBB modifications, sequencing of single cell clones will be also performed. PCR amplicons of the HBB locus from different cell sub-populations will be cloned into a standard TOPO vector and Sanger sequenced. Potential off-target sites will be predicted using the bioinformatics tool COSMID (70), and profiled using the genome-wide analysis tool Guide-seq (71). Targeted deep sequencing at off-target loci will be performed using the Illumina MiSeq platform to quantify levels of indel formation. A LAM-PCR based method will be used to determine if the AAV genome integrates into the genome. Thorough analysis will be performed on any identified integration sites to determine if they are true off-target sites.

Finally, to evaluate the potential therapeutic benefit of in vivo HBB editing for reversing SCD phenotypes, we will assess hematologic parameters (RBC, WBC and platelet number, Hb content, reticulocyte count, etc.), quantify RBC half-life and the presence of sickle cells in blood smears, determine spleen weight, assess kidney function (urine concentration, proteinuria, BUN/creatinine) and perform histologic analysis of spleens, livers and kidneys from AAV-HBB-CRISPR treated versus control treated (Cas9 only, gRNA only and donor+gRNA only) mice, as in (18, 72)). All analyses will be performed by an observer blinded to sample identity.

### BIBLIOGRAPHY

1. Dever et al, CRISPR/Cas9 beta-globin gene targeting in human haematopoietic stem cells. Nature. 2016; 539(7629):384-9.
2. Tabebordbar et al, In vivo gene editing in dystrophic mouse muscle and muscle stem cells. Science. 2015.
3. Chew et al, A multifunctional AAV-CRISPR-Cas9 and its host response. Nat Methods. 2016; 13(10):868-74.
4. Kean et al, Chimerism and cure: hematologic and pathologic correction of murine sickle cell disease. Blood. 2003; 102(13):4582-93.
5. Daya S, Berns K I. Gene therapy using adeno-associated virus vectors. Clinical microbiology reviews. 2008; 21(4):583-93.
6. Kohn D B, Pai S Y, Sadelain M. Gene therapy through autologous transplantation of gene-modified hematopoietic stem cells. Biol Blood Marrow Transplant. 2013; 19(1 Suppl):564-9.
7. Tolar J, Mehta P A, Walters M C. Hematopoietic cell transplantation for nonmalignant disorders. Biol Blood Marrow Transplant. 2012; 18(1 Suppl):5166-71.
8. Cavazzana-Calvo et al, Transfusion independence and HMGA2 activation after gene therapy of human beta-thalassaemia. Nature. 2010; 467(7313):318-22.
9. Hoban et al, Correction of the sickle cell disease mutation in human hematopoietic stem/progenitor cells. Blood. 2015; 125(17):2597-604.
10. Urbinati et al, Potentially therapeutic levels of anti-sickling globin gene expression following lentivirus-mediated gene transfer in sickle cell disease bone marrow CD34+ cells. Exp Hematol. 2015; 43(5):346-51.
11. Voit et al, Nuclease-mediated gene editing by homologous recombination of the human globin locus. Nucleic Acids Res. 2014; 42(2):1365-78.
12. Genovese et al, Targeted genome editing in human repopulating haematopoietic stem cells. Nature. 2014; 510(7504):235-40.
13. Kean et al, Comparison of mechanisms of anemia in mice with sickle cell disease and beta-thalassemia: peripheral destruction, ineffective erythropoiesis, and phospholipid scramblase-mediated phosphatidylserine exposure. Exp Hematol. 2002; 30(5):394-402.
14. Eldor A, Rachmilewitz E A. The hypercoagulable state in thalassemia. Blood. 2002; 99(1):36-43.
15. Cheng J K, Alper H S. The genome editing toolbox: a spectrum of approaches for targeted modification. Current opinion in biotechnology. 2014; 30C:87-94.
16. Seita J, Weissman I L. Hematopoietic stem cell: self-renewal versus differentiation. Wiley interdisciplinary reviews Systems biology and medicine. 2010; 2(6):640-53.
17. Wang et al, Homology-driven genome editing in hematopoietic stem and progenitor cells using ZFN mRNA and AAV6 donors. Nature biotechnology. 2015; 33(12):1256-63.
18. Wu et al, Correction of sickle cell disease by homologous recombination in embryonic stem cells. Blood. 2006; 108(4):1183-8.
19. Levasseur et al, Correction of a mouse model of sickle cell disease: lentiviral/antisickling beta-globin gene transduction of unmobilized, purified hematopoietic stem cells. Blood. 2003; 102(13):4312-9.
20. Nagel R L, et al, Structural bases of the inhibitory effects of hemoglobin F and hemoglobin A2 on the polymerization of hemoglobin S. Proc Natl Acad Sci USA. 1979; 76(2):670-2.
21. Oh vExpression of an anti-sickling beta-globin in human erythroblasts derived from retrovirally transduced primitive normal and sickle cell disease hematopoietic cells. Exp Hematol. 2004; 32(5):461-9.
22. Pawliuk et al, Correction of sickle cell disease in transgenic mouse models by gene therapy. Science. 2001; 294(5550):2368-71.
23. Urnov et al, Highly efficient endogenous human gene correction using designed zinc-finger nucleases. Nature. 2005; 435(7042):646-51.
24. Voit vGeneration of an HIV resistant T-cell line by targeted “stacking” of restriction factors. Molecular therapy: the journal of the American Society of Gene Therapy. 2013; 21(4):786-95. Epub 2013/01/30.
25. Sun et al, Clonal dynamics of native haematopoiesis. Nature. 2014; 514(7522):322-7.
26. Busch et al, Fundamental properties of unperturbed haematopoiesis from stem cells in vivo. Nature. 2015; 518(7540):542-6.
27. Gaj T, Gersbach C A, Barbas C F, 3rd. ZFN, TALEN, and CRISPR/Cas-based methods for genome engineering. Trends in biotechnology. 2013; 31(7):397-405.
28. Bogdanove A J, Voytas D F. TAL effectors: customizable proteins for DNA targeting. Science. 2011; 333(6051):1843-6.
29. Hendel et al, Quantifying genome-editing outcomes at endogenous loci with SMRT sequencing. Cell reports. 2014; 7(1):293-305.
30. Beard et al, Efficient and stable MGMT-mediated selection of long-term repopulating stem cells in nonhuman primates. J Clin Invest. 2010; 120(7):2345-54.
31. Phaltane et al, Efficiency and safety of 0(6)-methylguanine DNA methyltransferase (MGMT(P140K))-mediated in vivo selection in a humanized mouse model. Hum Gene Ther. 2014; 25(2):144-55.
32. Roth et al, MGMT enrichment and second gene co-expression in hematopoietic progenitor cells using separate or dual-gene lentiviral vectors. Virus research. 2014; 196C:170-80.
33. Gori et al, In vivo selection of autologous MGMT gene-modified cells following reduced-intensity conditioning with BCNU and temozolomide in the dog model. Cancer gene therapy. 2012; 19(8):523-9.
34. Adair et al, Extended survival of glioblastoma patients after chemoprotective HSC gene therapy. Sci Transl Med. 2012; 4(133):133ra57.
35. Eid et al, Real-time DNA sequencing from single polymerase molecules. Science. 2009; 323(5910):133-8.
36. Gao et al, Clades of Adeno-associated viruses are widely disseminated in human tissues. Journal of virology. 2004; 78(12):6381-8.
37. Boutin et al, Prevalence of serum IgG and neutralizing factors against adeno-associated virus (AAV) types 1, 2, 5, 6, 8, and 9 in the healthy population: implications for gene therapy using AAV vectors. Hum Gene Ther. 2010; 21(6):704-12.
38. Kotterman M A, Schaffer D V. Engineering adeno-associated viruses for clinical gene therapy. Nature reviews Genetics. 2014; 15(7):445-51.
39. Mingozzi F, High K A. Therapeutic in vivo gene transfer for genetic disease using AAV: progress and challenges. Nature reviews Genetics. 2011; 12(5):341-55.
40. Mali P, Esvelt K M, Church G M. Cas9 as a versatile tool for engineering biology. Nat Methods. 2013; 10(10):957-63.
41. Damian M, Porteus M H. A crisper look at genome editing: RNA-guided genome modification. Mol Ther. 2013; 21(4):720-2.
42. Ran et al, In vivo genome editing using Staphylococcus aureus Cas9. Nature. 2015; 520(7546):186-91.
43. Ran et al, Genome engineering using the CRISPR-Cas9 system. Nature protocols. 2013; 8(11):2281-308. Epub 2013/10/26.
44. Mali et al, RNA-guided human genome engineering via Cas9. Science. 2013; 339(6121):823-6.
45. Cong et al, Multiplex genome engineering using CRISPR/Cas systems. Science. 2013; 339(6121):819-23.
46. Friedland et al, Heritable genome editing in C. elegans via a CRISPR-Cas9 system. Nat Methods. 2013; 10(8):741-3. Epub 2013/07/03.
47. DiCarlo et al, Genome engineering in Saccharomyces cerevisiae using CRISPR-Cas systems. Nucleic Acids Res. 2013; 41(7):4336-43. Epub 2013/03/06.
48. Hwang et al, Efficient genome editing in zebrafish using a CRISPR-Cas system. Nature biotechnology. 2013; 31(3):227-9. Epub 2013/01/31.
49. Yin et al, Genome editing with Cas9 in adult mice corrects a disease mutation and phenotype. Nature biotechnology. 2014; 32(6):551-3. Epub 2014/04/01.
50. Ding et al, Permanent Alteration of PCSK9 With In Vivo CRISPR-Cas9 Genome Editing. Circ Res. 2014. Epub 2014/06/12.
51. Long et al, Postnatal genome editing partially restores dystrophin expression in a mouse model of muscular dystrophy. Science. 2015.
52. Nelson et al, In vivo genome editing improves muscle function in a mouse model of Duchenne muscular dystrophy. Science. 2015.
53. Madisen et al, A robust and high-throughput Cre reporting and characterization system for the whole mouse brain. Nat Neurosci. 2010; 13(1):133-40.
54. Zincarelli et al, Analysis of AAV serotypes 1-9 mediated gene expression and tropism in mice after systemic injection. Mol Ther. 2008; 16(6):1073-80. Epub 2008/04/17.
55. Le Hir et al, AAV genome loss from dystrophic mouse muscles during AAV-U7 snRNA-mediated exon-skipping therapy. Mol Ther. 2013; 21(8):1551-8.
56. Yang et al, A dual AAV system enables the Cas9-mediated correction of a metabolic liver disease in newborn mice. Nature biotechnology. 2016.
57. Deverman et al, Cre-dependent selection yields AAV variants for widespread gene transfer to the adult brain. Nature biotechnology. 2016.
58. Wright et al, Cyclophosphamide/granulocyte colony-stimulating factor causes selective mobilization of bone marrow hematopoietic stem cells into the blood after M phase of the cell cycle. Blood. 2001; 97(8):2278-85.
59. Wagers et al, Changes in integrin expression are associated with altered homing properties of Lin(−/lo)Thy1.1(10)Sca-1(+)c-kit(+) hematopoietic stem cells following mobilization by cyclophosphamide/granulocyte colony-stimulating factor. Exp Hematol. 2002; 30(2):176-85. PubMed PMID: 11823053.
60. Passegue et al, Global analysis of proliferation and cell cycle gene expression in the regulation of hematopoietic stem and progenitor cell fates. J Exp Med. 2005; 202(11):1599-611. PubMed PMID: 16330818.
61. Mandal et al, Efficient Ablation of Genes in Human Hematopoietic Stem and Effector Cells using CRISPR/Cas9. Cell Stem Cell. 2014; 15(5):643-52. doi: 10.1016/j.stem.2014.10.004. PubMed PMID: 25517468; PMCID: 4269831.
62. Zaiss et al, Differential activation of innate immune responses by adenovirus and adeno-associated virus vectors. Journal of virology. 2002; 76(9):4580-90. PubMed PMID: 11932423; PMCID: 155101.
63. Rossi et al, Cell intrinsic alterations underlie hematopoietic stem cell aging. Proc Natl Acad Sci USA. 2005; 102(26):9194-9. PubMed PMID: 15967997.
64. Min et al, The transcription factor EGR1 controls both the proliferation and localization of hematopoietic stem cells. Cell Stem Cell. 2008; 2(4):380-91. Epub 2008/04/10.
65. Rao et al, High-level Gpr56 expression is dispensable for the maintenance and function of hematopoietic stem and progenitor cells in mice. Stem Cell Res. 2015; 14(3):307-22.
66. et al, A cure for murine sickle cell disease through stable mixed chimerism and tolerance induction after nonmyeloablative conditioning and major histocompatibility complex-mismatched bone marrow transplantation. Blood. 2002; 99(5):1840-9. PubMed PMID: 11861303.
67. Lin et al, CRISPR/Cas9 systems have off-target activity with insertions or deletions between target DNA and guide RNA sequences. Nucleic acids research. 2014; 42(11):7473-85.
68. Muller et al, C. Streptococcus thermophilus CRISPR-Cas9 Systems Enable Specific Editing of the Human Genome. Mol Ther. 2015.
69. Lee C M, Cradick T J, Bao G. The Neisseria meningitidis CRISPR-Cas9 System Enables Specific Genome Editing in Mammalian Cells. Molecular therapy: the journal of the American Society of Gene Therapy. 2016.
70. Cradick T J, Qiu P, Lee C M, Fine E J, Bao G. COSMID: A Web-based Tool for Identifying and Validating CRISPR/Cas Off-target Sites. Molecular therapy Nucleic acids. 2014; 3:e214.
71. Tsai et al, GUIDE-seq enables genome-wide profiling of off-target cleavage by CRISPR-Cas nucleases. Nature biotechnology. 2015; 33(2):187-97.
72. Xu et al, Correction of sickle cell disease in adult mice by interference with fetal hemoglobin silencing. Science. 2011; 334(6058):993-6.
73. Smith et al, Strict in vivo specificity of the Bcl11a erythroid enhancer. Blood. 2016.
74. Platt et al, CRISPR-Cas9 knockin mice for genome editing and cancer modeling. Cell. 2014; 159(2):440-55.
75. Lu R, Neff N F, Quake S R, Weissman I L. Tracking single hematopoietic stem cells in vivo using high-throughput sequencing in conjunction with viral genetic barcoding. Nature biotechnology. 2011; 29(10):928-33.
76. Ogilvy et al, Promoter elements of vav drive transgene expression in vivo throughout the hematopoietic compartment. Blood. 1999; 94(6):1855-63. PubMed PMID: 10477714.
77. Almarza et al, Characteristics of lentiviral vectors harboring the proximal promoter of the vav proto-oncogene: a weak and efficient promoter for gene therapy. Mol Ther. 2007; 15(8):1487-94.
78. Gonzalez-Murillo et al, Development of lentiviral vectors with optimized transcriptional activity for the gene therapy of patients with Fanconi anemia. Hum Gene Ther. 2010; 21(5):623-30.
79. Smith et al, Gene transfer properties and structural modeling of human stem cell-derived AAV. Mol Ther. 2014; 22(9):1625-34.

In Situ Gene Editing to Discover New HSC Regulators

As shown herein, we have developed novel approaches to introduce programmable genetic lesions into endogenous blood stem cells, via adeno-associated virus (AAV)-mediated delivery of CRISPR/Cas9 gene editing complexes, as a means of interrogating gene function. Here, we propose to apply this system to rapidly and systematically interrogate novel candidate regulators that may control HSC self-renewal and differentiation functions. As outlined below, these studies will first optimize the AAV-CRISPR platform (through comparison of different viral serotypes and titers) by targeting known genes linked to dysregulated hematopoiesis and hematopoietic malignancy in mouse and human (xenograft) models (Aim 1). The studies will then be extended to a small pilot screen in which a panel of candidate genes whose expression is regulated concomitantly with variations in HSC self-renewal activity [1-3], but whose functional importance has not been evaluated will be examined (Aim 2). Results obtained from these studies will have both fundamental and translational importance for HSC biology and hematological disease by establishing a new experimental system for in situ genome manipulation of HSCs in their endogenous niche and identifying new mechanisms and mediators of HSC self-renewal and hematopoietic function.

A key innovation and advantage in the approach discussed herein is that manipulation of HSC gene expression can be accomplished in a highly programmable manner and without the need to remove HSC cells from their endogenous niche, thereby preserving their native regulatory interactions and extant stem cell properties. In addition, mutations will be introduced at relatively low frequencies (<10% of the total HSCs), allowing identification of targets whose manipulation can drive selective expansion of endogenous stem cells, as opposed to those that may provide a selective advantage only in transplant assays (which model a more regenerative, as opposed to homeostatic state, of the blood system). This in vivo AAV-CRISPR approach also overcomes a key challenge in typical transgenic and knockout-based models for assessing HSC gene function. Such conventional genetic systems require substantial time and resources to generate and have limited ability to evaluate combinatorial effects from disrupting multiple genes, particularly among linked loci. This AAV-CRISPR strategy circumvents such concerns by establishing a novel, robust system to discover and interrogate key genes and pathways that control HSC function, both singly and in combination. Finally, this transgene-independent approach is also uniquely amenable to interrogating xenografted human cells, and thus holds significant translational potential. With these key advantages, this study is expected to uncover novel stem cell biology.

The proposed approach is robustly supported by key published [4] and preliminary data demonstrating the feasibility and efficacy of delivering DNA modifying complexes by AAV to enable permanent gene-editing modifications within endogenous tissue stem cell populations, including long-term reconstituting HSCs. Preliminary results indicate that HSCs in normal adult mice are effectively transduced by multiple AAV serotypes by either intravenous or intrafemoral injection, and that this is effective in delivering sequence-specific gene editing nucleases. Importantly, subsequent transplantation of endogenously gene-modified cells has confirmed long-term, multi-lineage persistence of gene-edited hematopoietic cells and HSCs in recipient animals, suggesting that AAV transduction and gene editing do not disrupt the normal regenerative properties of HSCs. These data provide a sound technical basis for the studies proposed below, which will further optimize this system and apply it to the systematic testing in mammals of novel candidate stem cell regulatory target genes.

These studies will identify new genes and pathways that regulate endogenous HSC numbers and activity, and establish new systems for interrogating gene function in HSCs. Thus, they are directly relevant to understanding stem cell self-renewal at a molecular level. These results may identify new ways to expand endogenous HSCs to support blood regeneration and may open a new clinical path for treating genetic blood diseases via therapeutic genome modification in endogenous stem and progenitor cells.

Specific Aims:

Aim 1. Establish Optimal Viral Serotypes and Titers for Disrupting Known Aging-Relevant Target Genes in Endogenous Mouse and Human (Xenografted) HSCs.

To uncover currently unrecognized regulators of HSC self-renewal, it is crucial to establish a rapid and effective system to interrogate how mutations in discrete genes affect blood stem cell phenotypes. This novel in vivo gene-editing system provides a unique opportunity to harness CRISPR/Cas9 technology to introduce mutations into endogenous bone marrow HSCs and ask whether these mutations alter normal stem cell number and/or function. To optimize this system, AAV will be used to deliver CRISPR/Cas9 gene editing complexes targeting a known HSC self-renewal factor (Dnmt3a, [5]) into normal C57BL/6J or “humanized” NSGw41 mice (transplanted with human CD34+ progenitor cells) (FIG. 42). Cas9-based targeting strategies to disrupt this gene are already available from published ex vivo studies [5]. The efficiency of Dnmt3a gene disruption will be determined by next generation DNA sequencing and effects on HSC expansion assessed by quantification of mutation frequency, using an established in-house analysis pipeline (not shown), in isolated HSCs and mature blood lineages (T, B, and myeloid) at 5 days vs. 5 weeks after AAV injection. Results will be compared for at least 3 different AAV serotypes and titers. A guide RNA targeting LacZ [6] will be used as an experimental control. At the conclusion of these studies, we will select the set of parameters that results in the highest mutation frequency among mouse and human HSCs for subsequent studies in Aim 2.

Aim 2. Apply Multiplexed Screening Strategies to Identify Gene Targets that Enhance Self-Renewal of Endogenous Human HSCs.

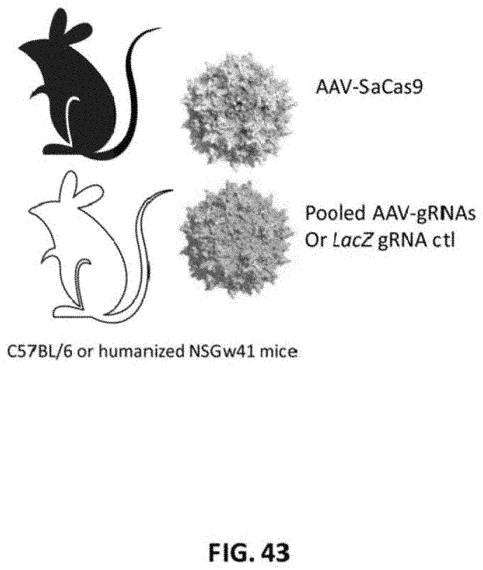

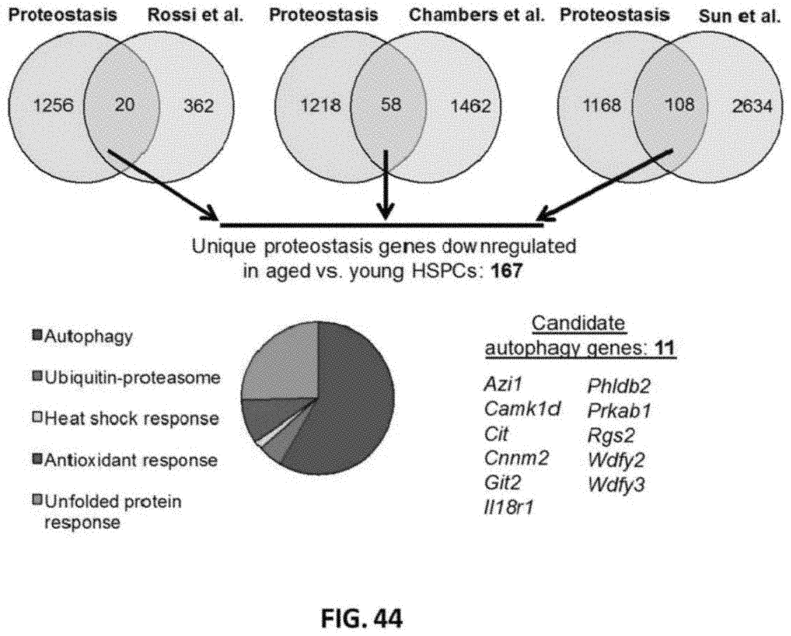

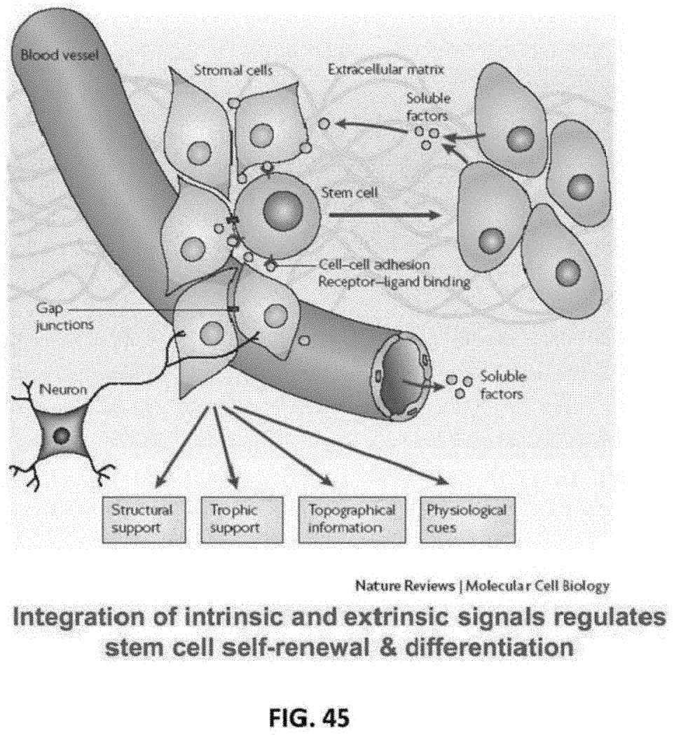

This aim will use immunophenotypic and functional assays to evaluate whether the acquisition of mutations in one of a set of 20 candidate regulators (identified from gene expression studies and with putative functions in biological processes, such as epigenetic regulation and proteostasis [7, 8], with previously demonstrated relevance to HSC self-renewal (see, e.g., FIG. 44) induces HSC expansion in vivo. Pooled AAVs, each containing Cas9 together with a single guide RNA (sgRNA) targeting one of each of these regulators, will be injected into normal C57BL/6J or “humanized” NSGw41 mice via systemic injection (FIG. 43). Viral serotype and titer will be determined by the studies in Aim 1. Each pool will contain up to 4 AAVs, and 2 gRNAs will be tested for each gene (in separate pools) to mitigate possible off-target effects. An sgRNA against LacZ will be used as a control. HSC expansion due to targeted gene disruption will be monitored as in Aim 1, by targeted next gen sequencing at each candidate modified locus. Together, the studies outlined in these two aims will develop AAV-CRISPR as a robust platform to identify novel genetic regulators of HSCs, using functional screening approaches in vivo and establish a pipeline for future screens of additional pathways and gene sets for their roles in regulating stem cell activity.

If none of the targeted gene disruptions planned for Aim 2 induce endogenous HSC expansion, we would then explore additional ways to perturb the blood system following gene targeting, such as sub-lethal irradiation, chemotherapy or high fat diet, to perhaps induce a change in HSC state, and we would also pursue multiplex strategies to target multiple genes simultaneously in these perturbation models and in the steady state.

AAV-CRISPR/Cas9 Mediates Disruption of an Endogenous Gene in the Genome of Endogenous Hematopoietic Stem Cells

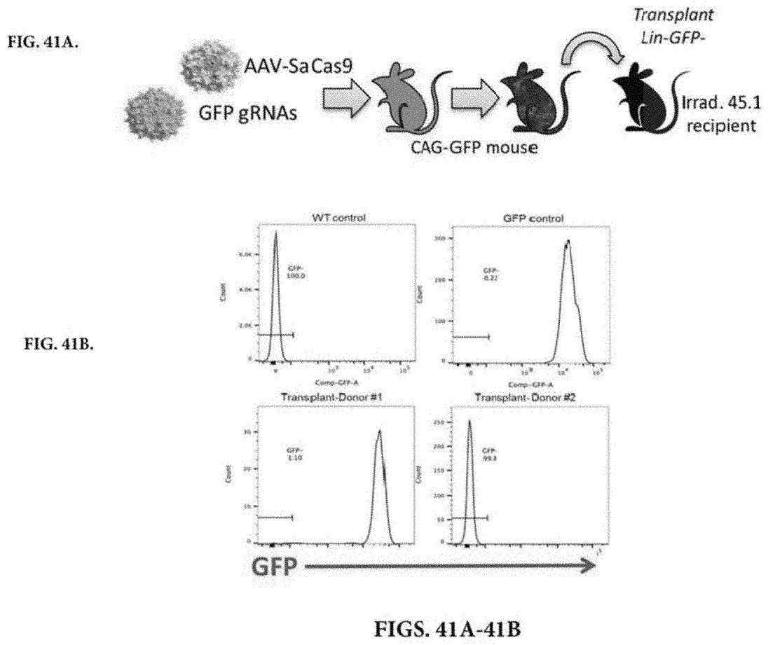

To demonstrate a two virus system for Cas9 mediated genetic modification of in vivo HSPCs, hemizygous CAAGS-eGFP mice, containing a single transgenic allele encoding ubiquitous GFP expression were injected with AAV-CRISPR particles (serotype 8) as well as AAV-gRNA particles targeting disruption of the GFP transgene. Three weeks later, bone marrow cells from the AAV-CRISPR injected mice were harvested and stained for a cocktail of lineage-specific antibodies: CD3, B220, Gr-1 and Ter119 (termed “Lineage”). Lineage low, GFP− bone marrow cells were isolated by Fluorescence Activated Cell Sorting (FACS) and transplanted into lethally irradiated CD45.1 recipient animals along with 3×10\hat{ }5 Sca1-depleted helper marrow cells of recipient allotype. (FIG. 41A)

Peripheral blood samples were collected from transplanted recipients and GFP expression was analyzed within donor-derived (CD45.2+) T, B and Myeloid cells based on expression of CD3, B220, Mac-1 and Gr-1. 1/3 of recipient mice showed multi-lineage hematopoietic reconstitution with donor-derived GFP− blood cells, indicating disruption in blood reconstituting hematopoietic stem and progenitor cells (HSPCs) of the genomically encoded GFP transgene by the AAV8-delivered gene editing complexes. In contrast, 100% of recipients of bone marrow cells from non-targeted mice showed engraftment with GFP+ cells. Data show peripheral blood cell analysis within live donor-derived at 8 weeks after transplant of WT (top left) and GFP control cells (top right) or cells from AAV8-CRISPR injected mice (bottom), including one animal reconstituted by non-disrupted (GFP+) donor-derived HSPCs (bottom left) and one reconstituted by disrupted (GFP−) donor-derived HSPCs (bottom right). (FIG. 41B)

Marker System for Genome Modification of Human HSC/HSPCs.

Our studies in mouse cells have been facilitated by our development of a reporter system for CRISPR/Cas9 activity. In this system, we co-deliver gRNAs targeting an engineered reporter gene locus (the Ai9 locus) with our gRNAs targeting our desired genomic locus and the Cas9 nuclease. These Ai9 gRNAs target sequences near the loxP sites that flank an upstream STOP cassette in the Ai9 locus, such that their assembly into active CRISPR complexes leads to excision of the intervening DNA, including the stop codon, and subsequent expression of the downstream TdTomato fluorescent reporter allele. We link the Ai9 reporter gRNAs with the gRNA(s) targeting the gene-of-interest, such that any cell that turns red (due to TdTomato expression) will have received the Ai9 gRNAs AND the gene-of-interest gRNAs AND an active Cas9 nuclease. Thus, we can use TdTomato expression as a surrogate to monitor the exposure of individual cells to active gene editing complexes (using flow cytometry to determine the frequency of TdTomato+ cells) and to purify cells that have been exposed to such (by FACS to sort out TdTomato+ cells). See FIG. 41A, FIG. 42 and Tabebordbar et al., Science 2016 for further details.

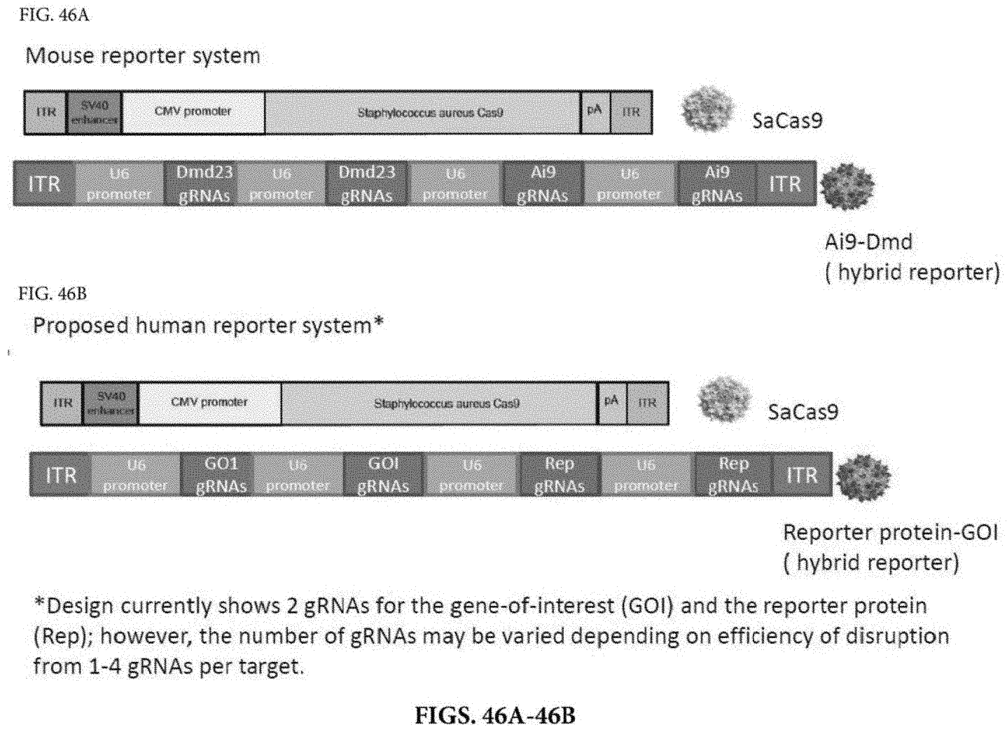

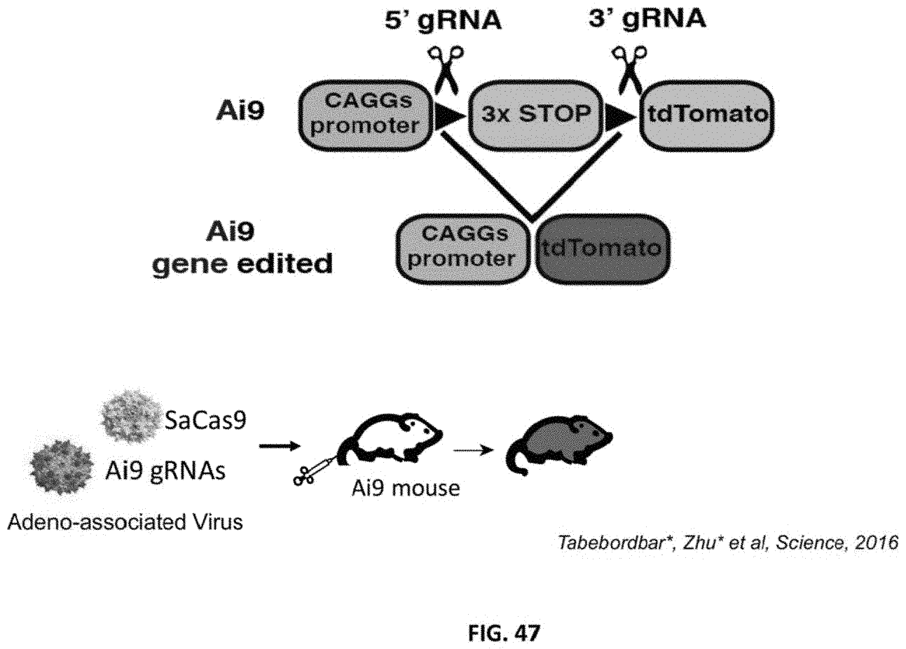

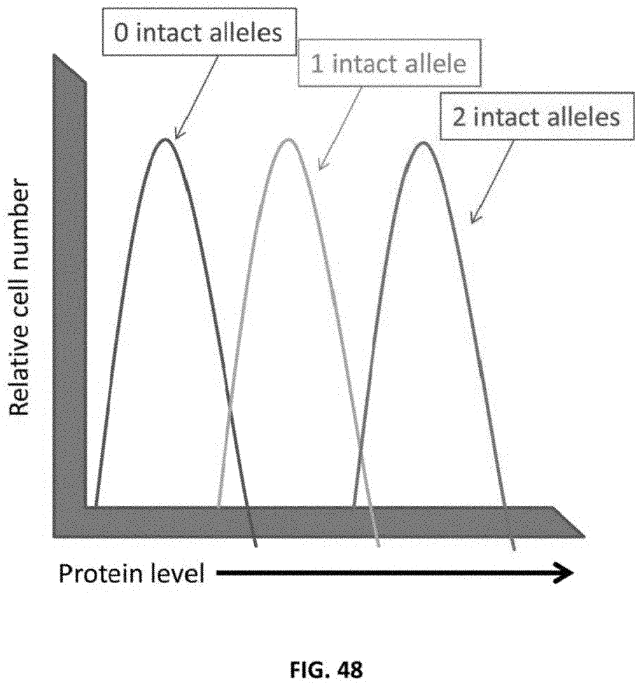

A similar reporter system to monitor gene editing rates and purify gene edited HUMAN cells is desirable, but of course the transgenic Ai9 system is inappropriate. As an alternative, we include linked gRNA(s) targeting a cell surface expressed molecule whose loss is non-pathogenic, and which exhibits gene dose-dependent levels of expression. In other words, complete loss of this molecule should not cause any phenotype, and the level of its expression on cells should be detectably and reproducibly HIGH in cells containing 2 intact copies of the gene encoding it, LOW in cells containing 1 intact copy and 1 disrupted copy, and ABSENT in cells containing 2 disrupted copies. Detection is accomplished by flow cytometry using an antibody specific to the reporter protein on blood cells, which can be obtained from human participants by simple blood draw. See FIG. 48. gRNAs targeting this human reporter would be linked in the AAV vector to gRNAs targeting the gene of interest, such that cells that show targeting of the reporter most likely also would be targeted at the gene-of-interest as well (FIG. 46B). Possible candidates for this reporter include but are not limited to: human CCR5 (HIV co-receptor).

### BIBLIOGRAPHY

1. Rossi, D. J., et al., Cell intrinsic alterations underlie hematopoietic stem cell aging. Proc Natl Acad Sci USA, 2005. 102(26): p. 9194-9.
2. Chambers, S. M., et al., Aging hematopoietic stem cells decline in function and exhibit epigenetic dysregulation. PLoS Biol, 2007. 5(8): p. e201.
3. Sun, D., et al., Epigenomic profiling of young and aged HSCs reveals concerted changes during aging that reinforce self-renewal. Cell Stem Cell, 2014. 14(5): p. 673-88.
4. Tabebordbar, M., et al., In vivo gene editing in dystrophic mouse muscle and muscle stem cells. Science, 2016. 351(6271): p. 407-11.
5. Gundry, M. C., et al., Highly Efficient Genome Editing of Murine and Human Hematopoietic Progenitor Cells by CRISPR/Cas9. Cell Rep, 2016. 17(5): p. 1453-1461.
6. Platt, R. J., et al., CRISPR-Cas9 knockin mice for genome editing and cancer modeling. Cell, 2014. 159(2): p. 440-55.
7. Beerman, I. and D. J. Rossi, Epigenetic Control of Stem Cell Potential during Homeostasis, Aging, and Disease. Cell Stem Cell, 2015. 16(6): p. 613-25.
8. Signer, R. A., et al., Haematopoietic stem cells require a highly regulated protein synthesis rate. Nature, 2014. 509(7498): p. 49-54.

1. A method for modifying the genome of one or more hematopoietic stem or progenitor cells (HSPCs) in a subject in vivo, comprising
a. contacting the subject with a virus, wherein the virus transduces a nucleic acid sequence encoding a sequence-targeting nuclease into the one or more HSPCs; and
b. modifying the genome of the one or more HSPCs with the sequence-targeting nuclease.

2. The method of claim 1, wherein the virus is adeno-associated virus (AAV) serotype 6, 8, 9 or 10.

3. (canceled)

4. The method of claim 1, wherein the virus is administered intravenously or is injected into bone marrow.

5. The method of claim 1, wherein the sequence-targeting nuclease is a Zinc-Finger Nuclease (ZFN), a Transcription activator-like effector nuclease (TALEN), or a Cas9 nuclease.

6. The method of claim 1, further comprising contacting the subject with a second virus which transduces a nucleic acid sequence encoding one or more gRNAs.

7. The method of claim 6, wherein the second virus is an AAV serotype 6, 8, 9 or 10.

8. (canceled)

9. The method of claim 1, wherein the genome of LT-HSCs are modified or preferentially modified, or wherein the genome of lineage restricted progenitor cells are modified or preferentially modified.

10. (canceled)

11. The method of claim 1, wherein the modification comprises the introduction or correction of a mutation associated with clonal hematopoiesis of indeterminate potential (CHIP), or wherein the modification comprises the introduction or correction of a mutation associated with Sickle cell disease (SCD) or β-thalassemia.

12.-15. (canceled)

16. The method of claim 1, wherein the modification comprises correction of a mutation via homology-directed repair.

17. (canceled)

18. (canceled)

19. A method for modifying a genetic region of interest in a cell in a subject in vivo, comprising
a. contacting the subject with a virus, wherein the virus transduces a nucleic acid sequence encoding a Cas9 nuclease into the cell;
b. contacting the subject with a second virus which transduces a nucleic acid sequence encoding a first set of one or more gRNAs targeting the genetic region of interest and a second set of one or more gRNAs targeting a genetic region encoding or controlling the expression of a cell surface marker;
c. modifying the genetic region of interest with the Cas9 nuclease; and
d. modulating expression of the cell surface marker.

20. The method of claim 19, wherein loss or gain of the cell surface marker by the cell is non-pathogenic.

21. The method of claim 19, further comprising detecting the likelihood or degree of modification of the genetic region of interest by detecting a change in the expression of the cell surface marker as compared to a control cell.

22. (canceled)

23. The method of claim 19, wherein the degree of modulation of the expression of the cell surface marker indicates whether one or both copies of a genetic region of interest are modified by the Cas9 nuclease.

24. The method of claim 19, wherein the cell surface marker is CCR5.

25. (canceled)

26. A method of screening for genetic regions coding for regulators of hematopoietic stem cell (HSC) self-renewal and/or differentiation, comprising
a. contacting an HSC in vivo with a virus, wherein the virus transduces a nucleic acid sequence encoding a sequence-targeting nuclease into the HSC;
b. modifying a genetic region of the HSC with the sequence targeting nuclease;
c. assessing the self-renewal and/or differentiation of the modified HSC;
wherein if modification of the genetic region modulates self-renewal and/or differentiation of the HSC then the genetic region is identified as coding for a regulator of hematopoietic stem cell (HSC) self-renewal and/or differentiation.

27. The method of claim 26, wherein the genetic region is a gene linked to dysregulated hematopoiesis and/or hematopoietic malignancy, or is linked to variations in HSC self-renewal activity.

28. The method of claim 26, wherein the virus is adeno-associated virus (AAV) serotype 6, 8, 9 or 10.

29. (canceled)

30. The method of claim 26, wherein the virus is administered intravenously or is injected into bone marrow.

31. The method of claim 26, wherein the sequence-targeting nuclease is a Zinc-Finger Nuclease (ZFN), a Transcription activator-like effector nuclease (TALEN), or a Cas9 nuclease.

32. The method of claim 26, further contacting the subject with a second virus which transduces a nucleic acid sequence encoding one or more gRNAs, wherein the one or more gRNA target the genetic region.

33.-36. (canceled)
